SEQUENCE LISTING (95 sequences)

SEQ ID NO: 1 - protein, 6 aa, Artificial Sequence
  Dalotuzumab HCDR1
GGYLWN

SEQ ID NO: 2 - protein, 16 aa, Artificial Sequence
  Dalotuzumab HCDR2
YISYDGTNNYKPSLKD

SEQ ID NO: 3 - protein, 8 aa, Artificial Sequence
  Dalotuzumab HCDR3
YGRVFFDY

SEQ ID NO: 4 - protein, 16 aa, Artificial Sequence
  Dalotuzumab LCDR1
RSSQSIVHSNGNTYLQ

SEQ ID NO: 5 - protein, 7 aa, Artificial Sequence
  Dalotuzumab LCDR2
KVSNRLY

SEQ ID NO: 6 - protein, 9 aa, Artificial Sequence
  Dalotuzumab LCDR3
FQGSHVPWT

SEQ ID NO: 7 - protein, 447 aa, Artificial Sequence
  Dalotuzumab Heavy Chain
QVQLQESGPGLVKPSETLSLTCTVSGYSITGGYLWNWIRQPPGKGLEWIGYISYDGTNNYKPSLKDRVTISRDTSKNQFSLKLSSVTAADTAVYYCARYGRVFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 219 aa, Artificial Sequence
  Dalotuzumab Light Chain
DIVMTQSPLSLPVTPGEPASISCRSSQSIVHSNGNTYLQWYLQKPGQSPQLLIYKVSNRLYGVPDRFSGSGSGTDFTLKISRVEAEDVGVYYCFQGSHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 6 aa, Artificial Sequence
  Ganitumab HCDR1
SSNWWS

SEQ ID NO: 10 - protein, 16 aa, Artificial Sequence
  Ganitumab HCDR2
EIYHSGSTNYNPSLKS

SEQ ID NO: 11 - protein, 10 aa, Artificial Sequence
  Ganitumab HCDR3
WTGRTDAFDI

SEQ ID NO: 12 - protein, 19 aa, Artificial Sequence
  Ganitumab LCDR1
ISCRSSQSLLHSNGYNYLD

SEQ ID NO: 13 - protein, 7 aa, Artificial Sequence
  Ganitumab LCDR2
LGSNRAS

SEQ ID NO: 14 - protein, 9 aa, Artificial Sequence
  Ganitumab LCDR3
MQGTHWPLT

SEQ ID NO: 15 - protein, 448 aa, Artificial Sequence
  Ganitumab Heavy Chain
QVQLQESGPGLVKPSGTLSLTCAVSGGSISSSNWWSWVRQPPGKGLEWIGEIYHSGSTNYNPSLKSRVTSVDKSKNQFSLKLSSVTAADTAVYYCARWTGRTDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 219 aa, Artificial Sequence
  Ganitumab Light Chain
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGTHWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 5 aa, Artificial Sequence
  Xentuzumab HCDR1
SYWMS

SEQ ID NO: 18 - protein, 15 aa, Artificial Sequence
  Xentuzumab HCDR2
SITSYGSFTYADSVK

SEQ ID NO: 19 - protein, 8 aa, Artificial Sequence
  Xentuzumab HCDR3
NMYTHFDS

SEQ ID NO: 20 - protein, 13 aa, Artificial Sequence
  Xentuzumab LCDR1
SGSSSNIGSNSVS

SEQ ID NO: 21 - protein, 7 aa, Artificial Sequence
  Xentuzumab LCDR2
DNSKRPS

SEQ ID NO: 22 - protein, 11 aa, Artificial Sequence
  Xentuzumab LCDR3
QSRDTYGYYWV

SEQ ID NO: 23 - protein, 400 aa, Artificial Sequence
  Xentuzumab Heavy Chain
QVELVESGGGLVQPGGSLRLSCAASGFTFTSYWMSWVRQAPGKGLELVSSITSYGSFTYYADSVKGRFTISRDNSKNTLYLQMNSLRAEDTAVYYCARNMYTHFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDS

SEQ ID NO: 24 - protein, 216 aa, Artificial Sequence
  Xentuzumab Light Chain
DIVLTQPPSVSGAPGQRVTISCSGSSSNIGSNSVSWYQQLPGTAPKLLIYDNSKRPSGVPDRFSGSKSGTSASLAITGLQSEDEADYYCQSRDTYGYYWVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 25 - protein, 5 aa, Artificial Sequence
  AVE1642 HCDR1
SYWMH

SEQ ID NO: 26 - protein, 17 aa, Artificial Sequence
  AVE1642 HCDR2
EINPSNGRTNYNEKFKR

SEQ ID NO: 27 - protein, 15 aa, Artificial Sequence
  AVE1642 HCDR3
GRPDYYGSSKWYFDV

SEQ ID NO: 28 - protein, 16 aa, Artificial Sequence
  AVE1642 LCDR1
RSSQSIVHSNVNTYLE

SEQ ID NO: 29 - protein, 7 aa, Artificial Sequence
  AVE1642 LCDR2
KVSNRFS

SEQ ID NO: 30 - protein, 9 aa, Artificial Sequence
  AVE1642 LCDR3
FQGSHVPPT

SEQ ID NO: 31 - protein, 124 aa, Artificial Sequence
  AVE1642 heavy chain (VH1)
QVQLQQSGAELVKPGASVKLSCKASGYTFTSYWMHWVKQRPGQGLEWIGEINPSNGRTNYNEKFKRKATLTVDKSSSTAYMQLSSLTSEDSAVYYFARGRPDYYGSSKWYFDVWGAGTTVTVSS

SEQ ID NO: 32 - protein, 113 aa, Artificial Sequence
  AVE1642 Light Chain (VL1)
DVLMTQTPLSLPVSLGDQASISCRSSQSIVHSNVNTYLEWYLQKPGQSPKLLIYKVSNRFSGVPDRFSGSGSGTDFTLRISRVEAEDLGIYYCFQGSHVPPTFGGGTKLEIKR

SEQ ID NO: 33 - protein, 10 aa, Artificial Sequence
  Figitumumab HCDR1
GFTFSSYAMN

SEQ ID NO: 34 - protein, 17 aa, Artificial Sequence
  Figitumumab HCDR2
AISGSGGTTFYADSVKG

SEQ ID NO: 35 - protein, 16 aa, Artificial Sequence
  Figitumumab HCDR3
DLGWSDSYYYYYGMDV

SEQ ID NO: 36 - protein, 11 aa, Artificial Sequence
  Figitumumab LCDR1
RASQGIRNDLG

SEQ ID NO: 37 - protein, 7 aa, Artificial Sequence
  Figitumumab LCDR2
AASRLHR

SEQ ID NO: 38 - protein, 9 aa, Artificial Sequence
  Figitumumab LCDR3
LQHNSYPCS

SEQ ID NO: 39 - protein, 451 aa, Artificial Sequence
  Figitumumab Heavy Chain (HC)
EVQLLESGGGLVQPGGSLRLSCTASGFTFSSYAMNWVRQAPGKGLEWVSAISGSGGTTFYADSVKGRFTISRDNSRTTLYLQMNSLRAEDTAVYYCAKDLGWSDSYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 40 - protein, 214 aa, Artificial Sequence
  Figitumumab Light Chain (LC)
DIQMTQFPSSLSASVGDRVTITCRASQGIRNDLGWYQQKPGKAPKRLIYAASRLHRGVPSRFSGSGSGTEFTLTISSLQPEDFATYYCLQHNSYPCSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 5 aa, Artificial Sequence
  Dusigitumab HCDR1
SYDIN

SEQ ID NO: 42 - protein, 17 aa, Artificial Sequence
  Dusigitumab HCDR2
WMNPNSGNTGYAQKFQG

SEQ ID NO: 43 - protein, 11 aa, Artificial Sequence
  Dusigitumab HCDR3
DPYYYYYGMDV

SEQ ID NO: 44 - protein, 13 aa, Artificial Sequence
  Dusigitumab LCDR1
SGSSSNIENNHVS

SEQ ID NO: 45 - protein, 7 aa, Artificial Sequence
  Dusigitumab LCDR2
DNNKRPS

SEQ ID NO: 46 - protein, 12 aa, Artificial Sequence
  Dusigitumab LCDR3
ETWDTSLSAGRV

SEQ ID NO: 47 - protein, 446 aa, Artificial Sequence
  Dusigitumab Heavy Chain (HC)
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCARDPYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 48 - protein, 217 aa, Artificial Sequence
  Dusigitumab Light Chain (LC)
QSVLTQPPSVSAAPGQKVTISCSGSSSNIENNHVSWYQQLPGTAPKLLIYDNNKRPSGIPDRFSGSKSGTSATLGITGLQTGDEADYYCETWDTSLSAGRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 49 - protein, 5 aa, Artificial Sequence
  Cixutumumab HCDR1
SYAIS

SEQ ID NO: 50 - protein, 16 aa, Artificial Sequence
  Cixutumumab HCDR2
GIIPIFGTANYAQKFQ

SEQ ID NO: 51 - protein, 21 aa, Artificial Sequence
  Cixutumumab HCDR3
APLRFLEWSTQDHYYYYYMDV

SEQ ID NO: 52 - protein, 11 aa, Artificial Sequence
  Cixutumumab LCDR1
QGDSLRSYYAT

SEQ ID NO: 53 - protein, 7 aa, Artificial Sequence
  Cixutumumab LCDR2
GENKRPS

SEQ ID NO: 54 - protein, 11 aa, Artificial Sequence
  Cixutumumab LCDR3
KSRDGSGQHLV

SEQ ID NO: 55 - protein, 460 aa, Artificial Sequence
  Cixutumumab Heavy Chain (HC)
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARAPLRFLEWSTQDHYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 214 aa, Artificial Sequence
  Cixutumumab Light Chain (LC)
SSELTQDPAVSVALGQTVRITCQGDSLRSYYATWYQQKPGQAPILVIYGENKRPSGIPDRFSGSSSGNTASLTITGAQAEDEADYYCKSRDGSGQHLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 57 - protein, 5 aa, Artificial Sequence
  BIIB022 HCDR1
IYRMQ

SEQ ID NO: 58 - protein, 17 aa, Artificial Sequence
  BIIB022 HCDR2
GISPSGGTTWYADSVKG

SEQ ID NO: 59 - protein, 11 aa, Artificial Sequence
  BIIB022 HCDR3
WSGGSGYAFDI

SEQ ID NO: 60 - protein, 10 aa, Artificial Sequence
  BIIB022 LCDR1
QASRDIRNYN

SEQ ID NO: 61 - protein, 7 aa, Artificial Sequence
  BIIB022 LCDR2
DASSLQT

SEQ ID NO: 62 - protein, 9 aa, Artificial Sequence
  BIIB022 LCDR3
QQFDSLPHT

SEQ ID NO: 63 - protein, 120 aa, Artificial Sequence
  BIIB022 Heavy Chain (HC)
EVQLLESGGGLVQPGGSLRLSCAASGFTFSIYRMQWVRQAPGKGLEWVSGISPSGGTTWYADSVKGRFTISRDNSKNTLYLQMNSLRAEDTAVYYCARWSGGSGYAFDIWGQGTMVTVSS

SEQ ID NO: 64 - protein, 107 aa, Artificial Sequence
  BIIB022 Light Chain (LC)
DIQMTQSPLSLSASVGDRVTITCQASRDIRNYLNWYQQKPGKAPKLLIYDASSLQTGVPSRFGGSGSGTDFSFTIGSLQPEDIATYYCQQFDSLPHTFGQGTKLEIK

SEQ ID NO: 65 - protein, 448 aa, Artificial Sequence
  Robatumumab Heavy Chain (HC)
EVQLVQSGGGLVKPGGSLRLSCAASGFTFSSFAMHWVRQAPGKGLEWISVIDTRGATYYADSVKGRFTISRDNAKNSLYLQMNSLRAEDTAVYYCARLGNFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 214 aa, Artificial Sequence
  Robatumumab Light Chain (LC)
EIVLTQSPGTLSVSPGERATLSCRASQSIGSSLHWYQQKPGQAPRLLIKYASQSLSGIPDRFSGSGSGTDFTLTISRLEPEDFAVYYCHQSSRLPHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 10 aa, Artificial Sequence
  Istiratumab HCDR1
GFMFSRYPMH

SEQ ID NO: 68 - protein, 16 aa, Artificial Sequence
  Istiratumab HCDR2
ISGSGGATPYADSVKG

SEQ ID NO: 69 - protein, 13 aa, Artificial Sequence
  Istiratumab HCDR3
DFYQILTGNAFDY

SEQ ID NO: 70 - protein, 11 aa, Artificial Sequence
  Istiratumab LCDR1
RASQGISSYLA

SEQ ID NO: 71 - protein, 7 aa, Artificial Sequence
  Istiratumab LCDR2
AKSTLQS

SEQ ID NO: 72 - protein, 9 aa, Artificial Sequence
  Istiratumab LCDR3
QQYWTFPLT

SEQ ID NO: 73 - protein, 720 aa, Artificial Sequence
  Istiratumab Heavy Chain (HC)
EVQLLQSGGGLVQPGGSLRLSCAASGFMFSRYPMHWVRQAPGKGLEWVGSISGSGGATPYADSVKGRFTISRDNSKNTLYLQMNSLRAEDTAVYYCAKDFYQILTGNAFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQVQLVQSGGGLVQPGGSLRLSCAASGFTFDDYAMHWVRQAPGKGLEWVAGISWDSGSTGYADSVKGRFTISRDNAKNSLYLQMNSLRAEDTALYYCARDLGAYQWVEGFDYWGQGTLVTVSSASTGGGGSGGGGSGGGGSGGGGSSYELTQDPAVSVALGQTVRITCQGDSLRSYYASWYQQKPGQAPVLVIYGKNNRPSGIPDRFSGSTSGNSASLTITGAQAEDEADYYCNSRDSPGNQWVFGGGTKVTVLG

SEQ ID NO: 74 - protein, 214 aa, Artificial Sequence
  Istiratumab Light Chain (LC)
DIQMTQSPSSLSASLGDRVTITCRASQGISSYLAWYQQKPGKAPKLLIYAKSTLQSGVPSRFSGSGSGTDFTLTISSLQPEDSATYYCQQYWTFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 10 aa, Artificial Sequence
  AVE1642 Heavy Chain CDR
GYTFTSYWMH

SEQ ID NO: 76 - protein, 10 aa, Artificial Sequence
  AVE1642 Heavy Chain CDR
EINPSNGRTN

SEQ ID NO: 77 - protein, 17 aa, Artificial Sequence
  AVE1642 Heavy Chain CDR
EINPSNGRTNYNQKFQG

SEQ ID NO: 78 - protein, 124 aa, Artificial Sequence
  AVE1642 Heavy Chain (VH2)
QVQLVQSGAEVVKPGASVKLSCKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVSS

SEQ ID NO: 79 - protein, 123 aa, Artificial Sequence
  AVE1642 Heavy Chain (VH3)
QVQLVQSGAEVVKPGASVKLSCKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVS

SEQ ID NO: 80 - protein, 113 aa, Artificial Sequence
  AVE1642 Light Chain (VL2)
DVVMTQTPLSLPVSLGDPASISCRSSQSIVHSNVNTYLEWYLQKPGQSPRLLIYKVSNRFSGVPDRFSGSGAGTDFTLRISRVEAEDLGIYYCFQGSHVPPTFGGGTKLEIKR

SEQ ID NO: 81 - protein, 113 aa, Artificial Sequence
  AVE1642 Light Chain (VL3)
DVLMTQTPLSLPVSLGDPASISCRSSQSIVHSNVNTYLEWYLQKPGQSPKLLIYKVSNRFSGVPDRFSGSGAGTDFTLRISRVEAEDLGIYYCFQGSHVPPTFGGGTKLEIKR

SEQ ID NO: 82 - protein, 113 aa, Artificial Sequence
  AVE1642 Light Chain (VL4)
DVLMTQTPLSLPVSLGDPASISCRSSQSIVHSNVNTYLEWYLQKPGQSPRLLIYKVSNRFSGVPDRFSGSGAGTDFTLRISRVEAEDLGIYYCFQGSHVPPTFGGGTKLEIKR

SEQ ID NO: 83 - protein, 113 aa, Artificial Sequence
  AVE1642 Light Chain (VL5)
DVVMTQTPLSLPVSLGDPASISCRSSQSIVHSNVNTYLEWYLQKPGQSPKLLIYKVSNRFSGVPDRFSGSGAGTDFTLRISRVEAEDLGIYYCFQGSHVPPTFGGGTKLEIKR

SEQ ID NO: 84 - protein, 5 aa, Artificial Sequence
  teprotumumab CDRH1
SYGMH

SEQ ID NO: 85 - protein, 17 aa, Artificial Sequence
  teprotumumab CDRH2
IIWFDGSSTYYADSVRG

SEQ ID NO: 86 - protein, 9 aa, Artificial Sequence
  teprotumumab CDRH3
ELGRRYFDL

SEQ ID NO: 87 - protein, 11 aa, Artificial Sequence
  teprotumumab CDRL1
RASQSVSSYLA

SEQ ID NO: 88 - protein, 7 aa, Artificial Sequence
  teprotumumab CDRL2
DASKRAT

SEQ ID NO: 89 - protein, 10 aa, Artificial Sequence
  teprotumumab CDRL3
QQRSKWPPWT

SEQ ID NO: 90 - protein, 118 aa, Artificial Sequence
  teprotumumab VH
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSS

SEQ ID NO: 91 - protein, 108 aa, Artificial Sequence
  teprotumumab VL
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 92 - protein, 17 aa, Artificial Sequence
  Antibody 2 CDRH2
IIWFDGSSKYYGDSVKG

SEQ ID NO: 93 - protein, 7 aa, Artificial Sequence
  Antibody 2 CDRL2
DASNRAT

SEQ ID NO: 94 - protein, 118 aa, Artificial Sequence
  Antibody 2 VH
QVQLVESGGGVVQPGRSLRLSCAASGFTFSSYGMHWVRQAPGKGLEWMAIIWFDGSSKYYGDSVKGRFTISRDNSKNTLYLQMNSLRADDTAVYYCARELGRRYFDLWGRGTLVTVSS

SEQ ID NO: 95 - protein, 108 aa, Artificial Sequence
  Antibody 2 VL
EIVLTQSPATLSLSPGERATLSCRASQSVSSYLAWYQQKPGQAPRLLIYDASNRATGIPARFSGSGSGTDFTLTISSLEPEDFAVYYCQQRSKWPPWTFGQGTKVEIK

(19) United States
(12) Patent Application Publication
(10) Pub. No.: US 2024/0158516 A1
(43) Pub. Date: May 16, 2024

(54) METHODS FOR THE TREATMENT OF THYROID EYE DISEASE

(71) Applicant: Horizon Therapeutics Ireland DAC, Dublin 2 (IE)

(72) Inventor: Jeffrey W. SHERMAN, Lincolnshire, IL (US)

(21) Appl. No.: 18/548,484

(22) PCT Filed: Mar. 3, 2022
(86) PCT No.: PCT/US2022/018737

§ 371 Date: Aug. 30, 2023

Related U.S. Application Data
(60) Provisional application No. 63/156,320, filed on Mar. 3, 2021.

(51) Int. Cl. C07K 16/28; A61K 39/00; A61P 27/02

(52) U.S. Cl. CPC C07K 16/2863; A61P 27/02; A61K 2039/505; A61K 2039/545; C07K 2317/52; C07K 2317/565; C07K 2317/76

(57) ABSTRACT

Provided herein are methods of treating or reducing the severity of thyroid eye disease (TED), also known as thyroid-associated ophthalmopathy (TAO), or Graves' ophthalmopathy or orbitopathy (GO), as well as antibodies, or antigen binding fragments thereof, and pharmaceutical compositions comprising them, useful in the methods.

This application claims the benefit of U.S. Provisional Application No. 63/156,320, filed Mar. 3, 2021, the disclosure of which is hereby incorporated by reference as if written herein in its entirety.

Thyroid eye disease (TED), also known as thyroid-associated ophthalmopathy (TAO), Graves' ophthalmopathy or orbitopathy (GO), thyrotoxic exophthalmos, dysthyroid ophthalmopathy, and several other terms, is orbitopathy associated with thyroid dysfunction. TAO is divided into two types. Active TED, which typically lasts 1-3 years, is characterized by an ongoing autoimmune/inflammatory response in the soft tissues of the orbit. Active TED is responsible for the expansion and remodeling of the ocular soft tissues. The autoimmune/inflammatory response of active, or acute, TED spontaneously resolves and the condition transitions into inactive TED. Inactive, or chronic, TED is the term used to describe the long-term/permanent sequelae of active TED.

### BACKGROUND OF THE DISCLOSURE

The cause of TED is unknown. TED is typically associated with Graves' hyperthyroidism but can also occur as part of other autoimmune conditions that affect the thyroid gland and produce pathology in orbital and periorbital tissue, and, rarely, the pretibial skin (pretibial myxedema) or digits (thyroid acropachy). TED is an autoimmune orbitopathy in which the orbital and periocular soft tissues are primarily affected with secondary effects on the eye and vision. In TED, as a result of inflammation and expansion of orbital soft tissues, primarily eye muscles and adipose, the eyes are forced forward (bulge) out of their sockets a phenomenon termed proptosis or exophthalmos.

The annual incidence rate of TED has been estimated at 16 cases per 100,000 women and 2.9 cases per 100,000 men from a study based in one largely rural Minnesota community. There appears to be a female preponderance in which women are affected 2.5-6 times more frequently than men; however, severe cases occur more often in men than in women. In addition, most patients are aged 30-50 years, with severe cases appearing to be more frequent in those older than 50 years. Although most cases of TED do not result in loss of vision, this condition can cause vision-threatening exposure keratopathy, troublesome diplopia (double vision), and compressive dysthyroid optic neuropathy.

TED may precede, coincide with, or follow the systemic complications of dysthyroidism. The ocular manifestations of TED include upper eyelid retraction, lid lag, swelling, redness (erythema), conjunctivitis, and bulging eyes (exophthalmos or proptosis), chemosis, periorbital edema, and altered ocular motility with significant functional, social, and cosmetic consequences.

Many of the signs and symptoms of TED, including proptosis and ocular congestion, result from expansion of the orbital adipose tissue and periocular muscles. The adipose tissue volume increases owing in part to new fat cell development (adipogenesis) within the orbital fat. The accumulation of hydrophilic glycosaminoglycans, primarily hyaluronic acid, within the orbital adipose tissue and the perimysial connective tissue between the extraocular muscle fibers, further expands the fat compartments and enlarges the extraocular muscle bodies. Hyaluronic acid is produced by fibroblasts residing within the orbital fat and extraocular muscles, and its synthesis in vitro is stimulated by several cytokines and growth factors, including IL-1β, interferon-γ, platelet-derived growth factor, thyroid stimulating hormone (TSH) and insulin-like growth factor I (IGF-I).

TED is commonly considered to be the autoimmune orbital manifestation of Graves' Disease (GD). However, only approximately 30% of patients with Graves' hyperthyroidism manifest clinically relevant ocular pathology indicating there is mechanistic heterogeneity and differentiation between the conditions. The molecular mechanisms underlying TED remain unclear. It is accepted that the generation of autoantibodies that act as agonists on the thyroid-stimulating hormone receptor (TSHR) is responsible for Graves' hyperthyroidism. Pathogenic overstimulation of TSHR, leads to overproduction of thyroid hormones (T3 and T4) and accelerated metabolism of many tissues.

In active TED, autoantibodies trigger connective tissue and fat to expand, in part from stimulating excessive synthesis of hyaluronan. The expanded tissues are infiltrated with T and B cells, become inflamed, and are extensively remodeled. It has been suggested that TSHR might have some pathogenic role in the development of active TED. Indeed, a positive correlation has been found between anti-TSHR antibodies and the degree of TED activity. However, no definitive link has been established, and a proportion of TED patients remain euthyroid throughout the course of their disease.

Antibodies that activate the insulin-like growth factor I receptor (IGF-IR) have also been detected and implicated in active TED. Without being bound to any theory, it is believed that TSHR and IGF-IR form a physical and functional complex in orbital fibroblasts, and that blocking IGF-IR appears to attenuate both IGF-I and TSH-dependent signaling. It has been suggested that blocking IGF-IR using an antibody antagonist might reduce both TSHR- and IGF-I-dependent signaling and therefore interrupt the pathological activities of autoantibodies acting as agonists on either receptor.

IGF-IR is a widely expressed heterotetrameric protein involved in the regulation of proliferation and metabolic function of many cell types. It is a tyrosine kinase receptor comprising two subunits. IGF-IRα contains a ligand-binding domain while IGF-IRβ is involved in signaling and contains tyrosine phosphorylation sites. Monoclonal antibodies directed against IGF-IR have been developed and assessed as a therapeutic strategy for several types of solid tumors and lymphomas.

Management of hyperthyroidism due to Graves' disease is imperfect because therapies targeting the specific underlying pathogenic autoimmune mechanisms of the disease are lacking. Even more complex is the treatment of moderate-to-severe active TED. Although recent years have witnessed a better understanding of its pathogenesis, TED remains a therapeutic challenge and dilemma. There are no approved drugs to treat active TED. Intravenous glucocorticoids (ivGCs) and oral glucocorticoids are used to treat patients with moderate-to-severe active TED, but results are seldom satisfactory. Partial responses are frequent and relapses (rebound) after drug withdrawal are not uncommon. Adverse events do occur and many patients eventually require rehabilitative surgery conducted when their condition has transitioned to inactive TED.

Recently, attention has been focused on the use of biologicals, which might specifically intervene on the pathogenic mechanisms of TED. In 2015 two small, monocenter, randomized clinical trials (RCTs) investigated the effects of rituximab, a CD20+ B cell-depleting agent, versus placebo or ivGCs, respectively. The results from the two trials were conflicting; they were negative (no differences with placebo) in the first trial, but positive (beneficial effects comparable to ivGCs) in the second one. The effectiveness of rituximab for moderate-to-severe active TED therefore remains to be determined. The recent guidelines published by the European Thyroid Association/European Group on Graves' Orbitopathy (EUGOGO) indicate rituximab as a possible second-line treatment for patients poorly responsive to a first course of ivGCs. As with rituximab, there is no dependable evidence concerning other potential therapeutic agents, such as adalimumab, etanercept, infliximab, or monoclonals or small molecules blocking the TSH receptor. The use of the interleukin-6 receptor monoclonal antibody, tocilizumab, based on an ongoing RCT also remains to be determined.

As stated above, medical therapies for moderate-to-severe TED that have proved to be effective and safe in adequately powered, prospective, placebo-controlled trials are lacking. Previous clinical trials, which were rarely placebo-controlled, suggest that high dose glucocorticoids, alone, or with radiotherapy, can reduce inflammation-related signs and symptoms in patients with active ophthalmopathy, but only minimally affect proptosis and can cause dose-limiting adverse reactions.

Immunoglobulins that activate IGF-IR signaling have been detected in patients with GD and TED. Furthermore, IGF-I synergistically enhances the actions of thyrotropin. IGF-IR, a membrane-spanning tyrosine kinase receptor with roles in development and metabolism, also stimulates immune function and thus might be targeted therapeutically in autoimmune diseases. IGF-IR is overexpressed by orbital fibroblasts and by T cells and B cells in persons with GD and TED. It forms a signaling complex with TSHR through which it is transactivated. In vitro studies of orbital fibroblasts and fibrocytes show that IGF-IR-inhibitory antibodies can attenuate the actions of IGF-I, thyrotropin, thyroid-stimulating immunoglobulins, and immunoglobulins isolated from patients with GD and TED. These observations prompted a trial of teprotumumab, a fully human IGF-IR-inhibitory monoclonal antibody, in patients with active, moderate-to-severe TED. Teprotumumab is currently the only pharmacologic therapy approved for the treatment of TED. Additional human IGF-IR-inhibitory monoclonal antibodies are still needed, including antibodies with extended half-life. Such half-life extended antibodies may have more convenient dosing, e.g. at lower volume and/or in lower amount and/or at lower frequency, and may be administrable either intravenously or subcutaneously.

### SUMMARY OF THE DISCLOSURE

Provided herein are methods of treating or reducing the severity of thyroid eye disease (TED), and achieving specific treatment endpoints in the treatment of TED, such as reducing proptosis, diplopia, TED clinical activity score and subsets and individual measures thereof, and of improving the quality of life of TED patients, comprising administering to the subject with TED an effective amount of an insulin like growth factor-I receptor (IGF 1R) inhibitor.

Certain IGF-1R inhibitors are able to decrease TSHR and IGF-IR display by orbital fibroblasts and fibrocytes and attenuate the actions of IGF-I, TSH, thyroid-stimulating immunoglobulins, and immunoglobulins isolated from patients with TED (TAO or GO).

As described above, TED (TAO or GO) remains inadequately treated. Prior to the approval of teprotumumab (TEPEZZA™), medical therapies, which primarily consisted of glucocorticoids, had limited efficacy and presented safety concerns. It is well known that broad immunosuppressive treatments for ED, e.g. glucocorticoids and rituximab, cause a limited reduction in exophthalmos. In the largest RCT using three different cumulative doses of ivGCs (2.25 g, 4.98 g, 7.47 g of methylprednisolone), the mean reduction in proptosis was 0.6 mm, even using the highest dose. Results were not different using rituximab. Further, advanced cases of TED (TAO or GO) usually called for more invasive surgical treatment such as orbital decompression. Previous therapies for the treatment of TED (TAO or GO) had, as stated above, not only limited efficacy, but also safety concerns. Teprotumumab, an IGF-1R inhibiting monoclonal antibody, has proven to be effective in the treatment of TED.

As stated by one of skill in the art, “[t]he most striking and unexpected effect of teprotumumab is the treatment-related decrease in exophthalmos [i.e., proptosis].” It is well known that immunosuppressive treatments for GO cause a limited reduction in exophthalmos, but with the methods disclosed herein] exophthalmos decreased by an average of 2.46 mm (vs. 0.15 mm in the placebo group). These results, never achieved with whatsoever medical treatment, are comparable to those obtained with orbital decompression” (Piantanida, E. and Bartalena, L. J Endocrinol Invest, 2017, 40, 885-887).

Although teprotumumab is effective for the treatment of TED, for various reasons, not all patients benefit from treatment with teprotumumab. There is still unmet medical need for alternate therapies for TED, e.g. for different drugs that may be administered via alternate modes and on alternate schedules.

### DETAILED DESCRIPTION

Provided herein are methods and compositions for the treatment of thyroid eye disease and related conditions, as illustrated by the following embodiments.

### EMBODIMENTS

Provided herein are:

Embodiment 1. A method of treating thyroid eye disease (TED), comprising administering to the subject an effective amount of an insulin like growth factor-I receptor (IGF-1R) inhibitor.

Embodiment 2. A method of reducing proptosis by at least 2 mm in a subject with thyroid eye disease (TED), comprising administering to the subject an effective amount of an IGF-IR inhibitor.

Embodiment 3. The method of Embodiment 2, wherein proptosis is reduced by at least 3 mm.

Embodiment 4. The method of Embodiment 3, wherein proptosis is reduced by at least 4 mm.

Embodiment 5. The method of Embodiment 2, wherein the method additionally comprises reducing the clinical activity score (CAS) in the subject with TED.

Embodiment 6. The method of Embodiment 5, wherein CAS is reduced by at least 2 points.

Embodiment 7. The method of Embodiment 6, wherein CAS is reduced by at least 3 points.

Embodiment 8. The method of Embodiment 7, wherein proptosis is reduced by at least 3 mm and CAS is reduced by at least 3 points.

Embodiment 9. A method of treating or reducing the severity of diplopia in a subject with thyroid eye disease (TED), comprising administering to the subject an effective amount of an insulin like growth factor-I receptor (IGF-1R) inhibitor.

Embodiment 10. The method of Embodiment 9, wherein the diplopia is constant diplopia.

Embodiment 11. The method of Embodiment 9, wherein the diplopia is intermittent diplopia.

Embodiment 12. The method of Embodiment 9, wherein the diplopia is inconstant diplopia

Embodiment 13. The method of any of Embodiments 9-12, wherein the improvement in or reduction in severity of diplopia is sustained at least 20 weeks after discontinuation of inhibitor administration.

Embodiment 14. The method of any of Embodiments 9-12, wherein the improvement in or reduction in severity of diplopia is sustained at least 50 weeks after discontinuation of inhibitor administration.

Embodiment 15. A method of treating or reducing the severity of thyroid eye disease (TED), or a symptom thereof, in a subject with TED, comprising administering to the subject an effective amount of an insulin like growth factor-I receptor (IGF-1R) inhibitor.

Embodiment 16. A method of reducing proptosis in an eye in a subject with thyroid eye disease (TED) in a subject with TED, comprising administering to the subject an effective amount of an insulin like growth factor-I receptor (IGF-1R) inhibitor.

Embodiment 17. A method of reducing Clinical Activity Score (CAS) of thyroid eye disease (TED) in a subject with TED, comprising administering to the subject an effective amount of an insulin like growth factor-I receptor (IGF-1R) inhibitor.

Embodiment 18. A method of a) reducing proptosis by at least 2 mm and b) reducing the clinical activity score (CAS) in a subject with thyroid eye disease (TED), comprising administering to the subject an effective amount of an insulin like growth factor-I receptor (IGF-1R) inhibitor.

Embodiment 19. The method of any of Embodiments 15, 16, and 18, wherein proptosis is reduced by at least 2 mm.

Embodiment 20. The method of Embodiment 19, wherein proptosis is reduced by at least 3 mm.

Embodiment 21. The method of Embodiment 20, wherein proptosis is reduced by at least 4 mm.

Embodiment 22. The method of any of Embodiments 15-21, wherein the clinical activity score (CAS) of the subject is reduced by at least 2 points.

Embodiment 23. The method of Embodiment 22, wherein the clinical activity score (CAS) of the subject is reduced to one (1).

Embodiment 24. The method of Embodiment 23, wherein the clinical activity score (CAS) of the subject is reduced to zero (0).

Embodiment 25. A method of improving the quality of life in a subject with thyroid eye disease (TED) comprising administering to the subject an effective amount of an insulin like growth factor-I receptor (IGF-1R) inhibitor.

Embodiment 26. The method of Embodiment 25, wherein the quality of life is measured by the Graves' Ophthalmopathy Quality of Life (GO-QoL) assessment, or either the Visual Functioning or Appearance subscale thereof.

Embodiment 27. The method of Embodiment 26, wherein the treatment results in an improvement of ≥8 points on the GO-QoL.

Embodiment 28. The method of Embodiment 26, wherein the treatment results in an improvement on the Functioning subscale of the GO-QoL.

Embodiment 29. The method of Embodiment 26, wherein the treatment results in an improvement on the Appearance subscale of the GO-QoL.

Embodiment 30. The method of any of Embodiments 1-29, wherein the TED is moderate-to-severe TED.

Embodiment 31. The method of any of Embodiments 1-30, wherein the TED is active/acute TED.

Embodiment 32. The method of any of Embodiments 1-30, wherein the TED is inactive/chronic TED.

Embodiment 33. The method of any of Embodiments 1-32, wherein the subject is a subject who has undergone prior treatment with an IGF-1R inhibitor and either did not respond to said prior treatment or relapsed after said prior treatment.

Embodiment 34. The method of any of Embodiments 1-33, wherein the treatment is efficacious for at least 20 weeks beyond the last administered dose.

Embodiment 35. The method of Embodiment 34, wherein the treatment is efficacious for at least 50 weeks beyond the last administered dose.

Embodiment 36. The method of any of Embodiments 1-35 wherein said IGF-1R inhibitor is an antibody or small molecule, with the proviso that the antibody is not teprotumumab.

Embodiment 37. The method of Embodiment 36 wherein said IGF-1R inhibitor is chosen from ganitumab, figitumumab, MEDI-573, cixutumumab, dalotuzumab, robatumumab, AVE1642, BIIB022, xentuzumab, istiratumab, linsitinib, picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, NSM-18, AZD3463, AZD9362, BI885578, BI893923, TT-100, XL-228, and A-928605.

Embodiment 38. The method of Embodiment 36 wherein said IGF-1R inhibitor is an antibody.

Embodiment 39. The method of Embodiment 37 wherein said IGF-1R inhibitor is a human, chimeric human, or humanized monoclonal antibody suitable for human therapy.

Embodiment 40. The method of Embodiment 38 wherein the antibody is administered intravenously (IV) or subcutaneously (SC).

Embodiment 41. The method of Embodiment 39 wherein the antibody is administered IV.

Embodiment 42. The method of Embodiment 40 wherein said antibody is chosen from ganitumab, figitumumab, MEDI-573, cixutumumab, dalotuzumab, robatumumab, AVE1642, BIIB022, xentuzumab, and istiratumab.

Embodiment 43. The method of Embodiment 42 wherein the antibody is ganitumab.

Embodiment 44. The method of Embodiment 43 wherein the ganitumab is dosed at:
a) 1-60 mg/kg or 75-4500 mg IV every 3 weeks; or
b) 0.6-40 mg/kg or 45-3000 mg IV every 2 weeks; or
c) 0.3-20 mg/kg; or 22-1500 mg IV weekly.

Embodiment 45. The method of Embodiment 42 wherein the antibody is figitumumab.

Embodiment 46. The method of Embodiment 45 wherein the figitumumab is dosed at:
a) 1-60 mg/kg or 75-4500 mg IV every 3 weeks; or
b) 0.6-40 mg/kg or 45-3000 mg IV every 2 weeks; or
c) 0.3-20 mg/kg or 22-1500 mg IV weekly.

Embodiment 47. The method of Embodiment 42 wherein the antibody is cixutumumab.

Embodiment 48. The method of Embodiment 47 wherein the cixutumumab is dosed at:
a) 1-45 mg/kg or 75-3400 mg IV every 3 weeks; or
b) 0.6-30 mg/kg or 45-2300 mg IV every 2 weeks; or
c) 0.3-15 mg/kg Or 22-1200 mg IV weekly.

Embodiment 49. The method of Embodiment 42 wherein the antibody is dalotuzumab.

Embodiment 50. The method of Embodiment 49 wherein the dalotuzumab is dosed at:
a) 1-90 mg/kg or 75-6800 mg IV every 3 weeks; or
b) 0.6-60 mg/kg or 45-4500 mg IV every 2 weeks; or
c) 0.3-30 mg/kg or 22-2300 mg IV weekly.

Embodiment 51. The method of Embodiment 42 wherein the antibody is robatumumab.

Embodiment 52. The method of Embodiment 51 wherein the robatumumab is dosed at:
a) 1-75 mg/kg or 75-5700 mg IV every 3 weeks; or
b) 0.6-50 mg/kg or 45-3800 mg IV every 2 weeks; or
c) 0.3-25 mg/kg or 22-1900 mg IV weekly.

Embodiment 53. The method of Embodiment 42 wherein the antibody is xentuzumab.

Embodiment 54. The method of Embodiment 53 wherein the xentuzumab is dosed at:
a) 1-112 mg/kg or 75-8400 mg IV every 3 weeks; or
b) 0.6-75 mg/kg or 45-5700 mg IV every 2 weeks; or
c) 0.3-38 mg/kg or 22-2900 mg IV weekly.

Embodiment 55. The method of Embodiment 42 wherein the antibody is istiratumab.

Embodiment 56. The method of Embodiment 55 wherein the istiratumab is dosed at:
a) 1-112 mg/kg or 75-8400 mg IV every 3 weeks; or
b) 0.6-75 mg/kg or 45-5700 mg IV every 2 weeks; or
c) 0.3-38 mg/kg or 22-2900 mg IV weekly.

Embodiment 57. The method of Embodiment 42 wherein the antibody is AVE1642.

Embodiment 58. The method of Embodiment 57 wherein the AVE1642 is dosed at:
a) 1-60 mg/kg or 75-4500 mg IV every 3 weeks; or
b) 0.6-40 mg/kg or 45-3000 mg IV every 2 weeks; or
c) 0.3-20 mg/kg or 22-1500 mg IV weekly.

Embodiment 59. The method of Embodiment 42 wherein the antibody is BIIB022.

Embodiment 60. The method of Embodiment 59 wherein the BIIB022 is dosed at:
a) 1-75 mg/kg or 75-5700 mg IV every 3 weeks; or
b) 0.6-50 mg/kg; or 45-3800 mg IV every 2 weeks; or
c) 0.3-25 mg/kg or 22-1900 mg IV weekly.

Embodiment 61. The method of Embodiment 48 wherein said IGF-1R inhibitor antibody comprises at least one heavy chain and at least one light chain selected from the selected from the group consisting of:
a) a heavy chain comprising the amino acid sequence of SEQ ID NO:7 and a light chain comprising the amino acid sequence SEQ ID NO:8;
b) a heavy chain comprising the amino acid sequence of SEQ ID NO:15 and a light chain comprising the amino acid sequence SEQ ID NO:16;
c) a heavy chain comprising the amino acid sequence of SEQ ID NO:23 and a light chain comprising the amino acid sequence SEQ ID NO:24;
d) a heavy chain comprising the amino acid sequence of SEQ ID NO:31 and a light chain comprising the amino acid sequence SEQ ID NO:32;
e) a heavy chain comprising the amino acid sequence of SEQ ID NO:39 and a light chain comprising the amino acid sequence SEQ ID NO:40;
f) a heavy chain comprising the amino acid sequence of SEQ ID NO:47 and a light chain comprising the amino acid sequence SEQ ID NO:48;
g) a heavy chain comprising the amino acid sequence of SEQ ID NO:55 and a light chain comprising the amino acid sequence SEQ ID NO:56;
h) a heavy chain comprising the amino acid sequence of SEQ ID NO:63 and a light chain comprising the amino acid sequence SEQ ID NO:64;
i) a heavy chain comprising the amino acid sequence of SEQ ID NO:65 and a light chain comprising the amino acid sequence SEQ ID NO:66; and
j) a heavy chain comprising the amino acid sequence of SEQ ID NO:73 and a light chain comprising the amino acid sequence SEQ ID NO:74.

Embodiment 62. The method of Embodiment 36 wherein said IGF-1R inhibitor is a small molecule.

Embodiment 63. The method of Embodiment 61 wherein said IGF-1R inhibitor is dosed orally.

Embodiment 64. The method of Embodiment 63 wherein said IGF-1R inhibitor is chosen from linsitinib, picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, NSM-18, AZD3463, AZD9362, BI885578, BI893923, TT-100, XL-228, and A-928605.

Embodiment 65. The method of Embodiment 64 wherein the IGF-1R inhibitor is linsitinib.

Embodiment 66. The method of Embodiment 65 wherein the linsitinib is dosed at:
a) 10-750 mg orally once daily continuous dosing or 10-1500 mg/day for once daily intermittent dosing (for up to 7 days of every 14 days); or
b) 6-500 mg orally twice daily continuous dosing or 6-1000 mg for twice daily intermittent dosing (for up to 7 days of every 14 days); or
c) 3-250 mg orally three-times daily continuous dosing or 3-500 mg for three-times daily intermittent dosing (for up to 7 days of every 14 days).

Embodiment 67. The method of Embodiment 64 wherein the IGF-1R inhibitor is picropodophyllin.

Embodiment 68. The method of Embodiment 67 wherein the picropodophyllin is dosed:
a) orally once daily at 20-2000 mg; or
b) orally twice daily at 13-1400 mg; or
c) orally three times daily at 6-700 mg.

Embodiment 69. The method of Embodiment 64 wherein the IGF-1R inhibitor is BMS-754807.

Embodiment 70. The method of Embodiment 69 wherein the BMS-754807 is dosed:
a) once daily at 5-600 mg orally; or
b) twice daily at 3-400 mg orally; or
c) three times daily at 1-200 mg.

Embodiment 71. The method of Embodiment 64 wherein the IGF-1R inhibitor is BMS-536924.

Embodiment 72. The method of Embodiment 64 wherein the IGF-1R inhibitor is BMS-554417.

Embodiment 73. The method of Embodiment 64 wherein the IGF-1R inhibitor is GSK1838705A.

Embodiment 74. The method of Embodiment 64 wherein the IGF-1R inhibitor is GSK1904529A.

Embodiment 75. The method of Embodiment 64 wherein the IGF-1R inhibitor is NVP-AEW541.

Embodiment 76. The method of Embodiment 64 wherein the IGF-1R inhibitor is NVP-ADW742.

Embodiment 77. The method of Embodiment 64 wherein the IGF-1R inhibitor is GTx-134.

Embodiment 78. The method of Embodiment 64 wherein the IGF-1R inhibitor is AG1024.

Embodiment 79. The method of Embodiment 64 wherein the IGF-1R inhibitor is PL-2258.

Embodiment 80. The method of Embodiment 64 wherein the IGF-1R inhibitor is NVP-AEW541.

Embodiment 81. The method of Embodiment 64 wherein the IGF-1R inhibitor is NSM-18.

Embodiment 82. The method of Embodiment 64 wherein the IGF-1R inhibitor is AZD3463.

Embodiment 83. The method of Embodiment 64 wherein the IGF-1R inhibitor is AZD9362.

Embodiment 84. The method of Embodiment 64 wherein the IGF-1R inhibitor is BI885578.

Embodiment 85. The method of Embodiment 64 wherein the IGF-1R inhibitor is BI893923.

Embodiment 86. The method of Embodiment 64 wherein the IGF-1R inhibitor is TT-100.

Embodiment 87. The method of Embodiment 64 wherein the IGF-1R inhibitor is XL-228.

Embodiment 80. The method of Embodiment 64 wherein the IGF-1R inhibitor is A-928605.

Embodiment 88. The method of any of Embodiments 71-88 wherein the IGF-1R inhibitor is dosed:
a) once daily at 1-2000 mg orally; or
b) twice daily at 0.6-1400 mg orally; or
c) three times daily at 0.3-700 mg orally.

Embodiment 89. The method of Embodiment 64 wherein the IGF-1R inhibitor is KW-2450.

Embodiment 90. The method of Embodiment 90 wherein the KW-2450 is dosed:
a) once daily at 1-100 mg orally; or
b) twice daily at 0.6-70 mg orally; or
c) three times daily at 0.3-30 mg orally.

Embodiment 91. The method of any of Embodiments 1-30 and 33-35, wherein the TED is inactive/chronic TED, and wherein the IGF-1R inhibitor is teprotumumab.

Also provided herein are pharmaceutical compositions for the treatment of TED comprising an IGF-1R inhibitor.

Embodiment 92. A pharmaceutical composition comprising an amount of an insulin like growth factor-I receptor (IGF-1R) inhibitor that is therapeutically effective:
for treating or reducing the severity of thyroid eye disease (TED) or a symptom thereof;
for reducing proptosis by at least 2 mm in a subject with thyroid eye disease (TED)
for treating or reducing the severity of diplopia in a subject with thyroid eye disease (TED);
for reducing Clinical Activity Score (CAS) of thyroid eye disease (TED);
for a) reducing proptosis by at least 2 mm and b) reducing the clinical activity score (CAS) in a subject with thyroid eye disease (TED); and/or
for improving the quality of life in a subject with thyroid eye disease (TED) wherein the quality of life is measured by the Graves' Ophthalmopathy Quality of Life (GO-QoL) assessment, or either the Visual Functioning or Appearance subscale thereof.

Embodiment 93. The pharmaceutical composition of Embodiment 92, wherein the IGF-1R inhibitor is ganitumab, formulated for administration:
a) 1-60 mg/kg or 75-4500 mg IV every 3 weeks; or
b) 0.6-40 mg/kg or 45-3000 mg IV every 2 weeks; or
c) 0.3-20 mg/kg; or 22-1500 mg IV weekly.

Embodiment 94. The pharmaceutical composition of Embodiment 92, wherein the IGF-1R inhibitor is figitumumab, formulated for administration:
a) 1-60 mg/kg or 75-4500 mg IV every 3 weeks; or
b) 0.6-40 mg/kg or 45-3000 mg IV every 2 weeks; or
c) 0.3-20 mg/kg or 22-1500 mg IV weekly.

Embodiment 95. The pharmaceutical composition of Embodiment 92, wherein the IGF-1R inhibitor is cixutumumab, formulated for administration:
a) 1-45 mg/kg or 75-3400 mg IV every 3 weeks; or
b) 0.6-30 mg/kg or 45-2300 mg IV every 2 weeks; or
c) 0.3-15 mg/kg Or 22-1200 mg IV weekly.

Embodiment 96. The pharmaceutical composition of Embodiment 92, wherein the IGF-1R inhibitor is dalotuzumab, formulated for administration:
a) 1-90 mg/kg or 75-6800 mg IV every 3 weeks; or
b) 0.6-60 mg/kg or 45-4500 mg IV every 2 weeks; or
c) 0.3-30 mg/kg or 22-2300 mg IV weekly.

Embodiment 97. The pharmaceutical composition of Embodiment 92, wherein the IGF-1R inhibitor is robatumumab, formulated for administration:
a) 1-75 mg/kg or 75-5700 mg IV every 3 weeks; or
b) 0.6-50 mg/kg or 45-3800 mg IV every 2 weeks; or
c) 0.3-25 mg/kg or 22-1900 mg IV weekly.

Embodiment 98. The pharmaceutical composition of Embodiment 92, wherein the IGF-1R inhibitor is xentuzumab, formulated for administration:
a) 1-112 mg/kg or 75-8400 mg IV every 3 weeks; or
b) 0.6-75 mg/kg or 45-5700 mg IV every 2 weeks; or
c) 0.3-38 mg/kg or 22-2900 mg IV weekly.

Embodiment 99. The pharmaceutical composition of Embodiment 92, wherein the IGF-1R inhibitor is istiratumab, formulated for administration:
a) 1-112 mg/kg or 75-8400 mg IV every 3 weeks; or
b) 0.6-75 mg/kg or 45-5700 mg IV every 2 weeks; or
c) 0.3-38 mg/kg or 22-2900 mg IV weekly.

Embodiment 100. The pharmaceutical composition of Embodiment 92, wherein the IGF-1R inhibitor is AVE1642, formulated for administration:
a) 1-60 mg/kg or 75-4500 mg IV every 3 weeks; or
b) 0.6-40 mg/kg or 45-3000 mg IV every 2 weeks; or
c) 0.3-20 mg/kg or 22-1500 mg IV weekly.

Embodiment 101. The pharmaceutical composition of Embodiment 92, wherein the IGF-1R inhibitor is BIIB022, formulated for administration at:
a) 1-75 mg/kg or 75-5700 mg IV every 3 weeks; or
b) 0.6-50 mg/kg; or 45-3800 mg IV every 2 weeks; or
c) 0.3-25 mg/kg or 22-1900 mg IV weekly.

Embodiment 102. The pharmaceutical composition of Embodiment 92, wherein the IGF-1R inhibitor is linsitinib, formulated for administration at:
a) 10-750 mg orally once daily continuous dosing or 10-1500 mg/day for once daily intermittent dosing (for up to 7 days of every 14 days); or
b) 6-500 mg orally twice daily continuous dosing or 6-1000 mg for twice daily intermittent dosing (for up to 7 days of every 14 days); or
c) 3-250 mg orally three-times daily continuous dosing or 3-500 mg for three-times daily intermittent dosing (for up to 7 days of every 14 days).

Embodiment 103. The pharmaceutical composition of Embodiment 92, wherein the IGF-1R inhibitor is picropodophyllin, formulated for administration:
a) orally once daily at 20-2000 mg; or
b) orally twice daily at 13-1400 mg; or
c) orally three times daily at 6-700 mg.

Embodiment 104. The pharmaceutical composition of Embodiment 92, wherein the IGF-1R inhibitor is BMS-754807, formulated for administration:
a) once daily at 5-600 mg orally; or
b) twice daily at 3-400 mg orally; or
c) three times daily at 1-200 mg orally.

Embodiment 105. The pharmaceutical composition of Embodiment 92, wherein the IGF-1R inhibitor is chosen from BMS-536924, BMS-554417, GSK1838705A, GSK1904529A, NVP-AEW541, NVP-ADW742, GTx-134, AG1024, PL-2258, NVP-AEW541, NSM-18, AZD3463, AZD9362, BI885578, BI893923, TT-100, XL-228, and A-928605, formulated for administration:
a) once daily at 1-2000 mg orally; or
b) twice daily at 0.6-1400 mg orally; or
c) three times daily at 0.3-700 mg orally.

Embodiment 106. The pharmaceutical composition of Embodiment 92, wherein the IGF-1R inhibitor is KW-2450, formulated for administration;
a) once daily at 1-100 mg orally; or
b) twice daily at 0.6-70 mg orally; or
c) three times daily at 0.3-30 mg orally.

Embodiment 107. The method of Embodiment 57, wherein the AVE1642 antibody comprises a HCDR1 comprising the amino acid sequence of SEQ ID NO:25, a HCDR2 comprising the amino acid sequence of SEQ ID NO:76, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30.

Embodiment 108. The method of Embodiment 107, wherein the antibody comprises a heavy chain variable domain comprising SEQ ID NO:31 or 78 or 79, and a light chain variable domain comprising SEQ ID NO:32 or 80 or 81 or 82 or 83.

Embodiment 109. The method of Embodiment 108, wherein the antibody comprises a heavy chain variable domain comprising SEQ ID NO:78 and a light chain variable domain comprising SEQ ID NO:80 or 81 or 82 or 83.

Embodiment 110. The method of Embodiment 109, wherein the antibody comprises the light chain variable domain comprising SEQ ID NO:80.

Embodiment 111. The method of Embodiment 109, wherein the antibody comprises the light chain variable domain comprising SEQ ID NO:81.

Embodiment 112. The method of Embodiment 109, wherein the antibody comprises the light chain variable domain comprising SEQ ID NO:82.

Embodiment 113. The method of Embodiment 109, wherein the antibody comprises the light chain variable domain comprising SEQ ID NO:83.

Embodiment 114. The method of any of Embodiments 107-113, wherein the therapeutically effective amount of the AVE1642 antibody comprises a dosage of 1-60 mg/kg or 75-4500 mg IV Q3W; or 0.6-40 mg/kg or 45-3000 mg IV Q2W; or 0.3-20 mg/kg or 22-1500 mg IV QW.

Embodiment 115. The method of any of Embodiments of 107-113, wherein the therapeutically effective amount of the AVE1642 antibody comprises a dosage of 1-10 mg/kg.

Embodiment 116. The method of Embodiment 115, wherein the therapeutically effective amount of the AVE1642 antibody comprises a dosage of 1-5 mg/kg.

Embodiment 117. The method of Embodiment 116, wherein the therapeutically effective amount of the AVE1642 antibody comprises a dosage of about 1 mg/kg, or about 2 mg/kg, or about 3 mg/kg, or about 4 mg/kg, or about 5 mg/kg.

Embodiment 118. The method of any of Embodiments 115-117, wherein the therapeutically effective amount of the AVE1642 antibody is administered every 1, 2, 3, 4, or 5 weeks (i.e., QW, Q2W, Q3W, Q4W, or Q5W).

Embodiment 119. The method of Embodiment 118, wherein the AVE1642 antibody is administered intravenously (IV) or subcutaneously (SC).

Embodiment 120. The method of any of Embodiments 107-113, wherein the therapeutically effective amount of the AVE1642 antibody comprises a dosage of 1-5 mg/kg or 75-375 mg IV Q3W; or 0.6-4 mg/kg or 45-300 mg IV Q2W; or 0.3-3 mg/kg or 22-225 mg IV QW.

Embodiment 121. The method of any of Embodiments 107-120, wherein the AVE1642 antibody further comprises a variant Fc region comprising mutations that substitute a methionine at position 428 with a leucine (Met428Leu) and substitute an asparagine at position 434 with a serine (Asn434Ser), wherein the amino acid substitution numbering is EU as in Kabat.

Embodiment 122. The method of any Embodiments 115-117, wherein the AVE1642 antibody further comprises a variant Fc region comprising mutations that substitute a methionine at position 428 with a leucine (Met428Leu) and substitute an asparagine at position 434 with a serine (Asn434Ser), wherein the amino acid substitution numbering is EU as in Kabat.

Embodiment 123. The method of Embodiment 122, wherein the therapeutically effective amount of the AVE1642 antibody is administered every 1, 2, 3, 4, 5, 6, 7, 8, 9, 10, 11, or 12 weeks (i.e., QW, Q2W, Q3W, Q4W, Q5W, Q6W, Q7W, Q8W, Q9W, Q10W, Q11W, or Q12W).

Embodiment 124. The method of Embodiment 123, wherein the AVE1642 antibody is administered intravenously (IV) or subcutaneously (SC).

Embodiment 125. The method of any of embodiments 1-35, wherein the IGF-1R inhibitor is the teprotumumab antibody further comprising a variant Fc region comprising mutations that substitute a methionine at position 428 with a leucine (Met428Leu) and substitute an asparagine at position 434 with a serine (Asn434Ser), wherein the amino acid substitution numbering is EU as in Kabat.

Embodiment 126. The method of any of embodiments 1-35, wherein the IGF-1R inhibitor is the teprotumumab antibody further comprising a variant Fc region comprising mutations that substitute a methionine at position 252 with a tyrosine (Met252Tyr), substitute a serine at position 254 with a threonine (Ser254Thr), and substitute a threonine at position 256 with a glutamic acid (Thr256Glu), wherein the amino acid substitution numbering is EU as in Kabat.

Embodiments 127-200 are intentionally omitted.

Embodiment 201. An antibody which binds to the insulin like growth factor-I receptor (IGF-1R), comprising either:
a variant Fc region comprising mutations that substitute a methionine at position 428 with a leucine (Met428Leu) and substitute an asparagine at position 434 with a serine (Asn434Ser), wherein the amino acid substitution numbering is EU as in Kabat; or
a variant Fc region comprising mutations that substitute a first mutation that is a tyrosine at position 252 (Met252Tyr), a second mutation that is a threonine at position 254 (Ser254Thr), and a third mutation that is a glutamic acid at position 256 (Thr256Glu), wherein the amino acid substitution numbering is EU as in Kabat.

Embodiment 202. The antibody of embodiment 201, wherein the antibody binds to and inhibits IGF-1R.

Embodiment 203. The antibody of either of embodiments 201-202, wherein the antibody or antigen-binding portion thereof cross-competes for binding to IGF-1R with a reference antibody or reference antigen-binding portion thereof.

Embodiment 204. The antibody of embodiment 203, wherein the reference antibody is chosen from αIR3, dalotuzumab, ganitumab, xentuzumab, AVE1642, figitumumab, dusigitumab, cituxumumab, BIIB022, robatumumab, teprotumumab, and Antibody 2.

Embodiment 205. The antibody of any of embodiments 201-204, wherein the antibody is chosen from an IgA, IgD, IgE, and IgG.

Embodiment 206. The antibody of embodiment 205, wherein the antibody is an IgG.

Embodiment 207. The antibody of embodiment 206, wherein the antibody is chosen from an IgG1, IgG2, IgG3, and IgG4.

Embodiment 208. The antibody of embodiment 207, wherein the antibody is an IgG1.

Embodiment 209. The antibody of any of embodiments 201-208, wherein the antibody:
is capable of reducing insulin like growth factor-I receptor (IGF-1R) signaling;
is capable of inhibiting thyroid stimulating hormone receptor (TSHR)/IGF-1R crosstalk (i.e., formation of a TSHR/IGF-1R signalosome);
is capable of reducing hyaluronan (HA) secretion in orbital fibroblasts;
is capable of persisting for an extended period of time in vivo (i.e., has a longer half-life) compared to an antibody that does not comprise the M428L/N434S or M252Y/S254T/T256E substitutions; and/or
is capable of being dosed less frequently, or in a lower amount per dose, compared to an antibody that does not comprise the M428L/N434S or M252Y/S254T/T256E substitutions.

Embodiment 210. The antibody of any of embodiments 201-209, wherein the antibody comprises complementarity determining regions (CDRs) derived from an antibody chosen from dalotuzumab, ganitumab, xentuzumab, AVE1642, figitumumab, dusigitumab, cituxumumab, BIIB022, robatumumab, teprotumumab, and Antibody 2.

Embodiment 211. The antibody of any of embodiments 201-210 wherein the antibody comprises a heavy chain variable region that comprises HCDR1, HCDR2, and HCDR3 domains; and a light chain variable region that comprises LCDR1, LCDR2, and LCDR3 domains, comprising:
a HCDR1 comprising the amino acid sequence of SEQ ID NO:1, a HCDR2 comprising the amino acid sequence of SEQ ID NO:2, a HCDR3 comprising the amino acid sequence of SEQ ID NO:3, a LCDR1 comprising the amino acid sequence of SEQ ID NO:4, a LCDR2 comprising the amino acid sequence of SEQ ID NO:5, and a LCDR3 comprising the amino acid sequence of SEQ ID NO:6;
a HCDR1 comprising the amino acid sequence of SEQ ID NO:9, a HCDR2 comprising the amino acid sequence of SEQ ID NO:10, a HCDR3 comprising the amino acid sequence of SEQ ID NO:11, a LCDR1 comprising the amino acid sequence of SEQ ID NO:12, a LCDR2 comprising the amino acid sequence of SEQ ID NO:13, and a LCDR3 comprising the amino acid sequence of SEQ ID NO:14;
a HCDR1 comprising the amino acid sequence of SEQ ID NO:17, a HCDR2 comprising the amino acid sequence of SEQ ID NO:18, a HCDR3 comprising the amino acid sequence of SEQ ID NO:19, a LCDR1 comprising the amino acid sequence of SEQ ID NO:20, a LCDR2 comprising the amino acid sequence of SEQ ID NO:21, and a LCDR3 comprising the amino acid sequence of SEQ ID NO:22;
a HCDR1 comprising HCDR1 comprising the amino acid sequence of SEQ ID NO:25 or SEQ ID NO:75, a HCDR2 comprising the amino acid sequence of SEQ ID NO:26 or SEQ ID NO:76 or SEQ ID NO:77, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30;
a HCDR1 comprising the amino acid sequence of SEQ ID NO:33, a HCDR2 comprising the amino acid sequence of SEQ ID NO:34, a HCDR3 comprising the amino acid sequence of SEQ ID NO:35, a LCDR1 comprising the amino acid sequence of SEQ ID NO:36, a LCDR2 comprising the amino acid sequence of SEQ ID NO:37, and a LCDR3 comprising the amino acid sequence of SEQ ID NO:38;
a HCDR1 comprising the amino acid sequence of SEQ ID NO:41, a HCDR2 comprising the amino acid sequence of SEQ ID NO:42, a HCDR3 comprising the amino acid sequence of SEQ ID NO:43, a LCDR1 comprising the amino acid sequence of SEQ ID NO:44, a LCDR2 comprising the amino acid sequence of SEQ ID NO:45, and a LCDR3 comprising the amino acid sequence of SEQ ID NO:46;
a HCDR1 comprising the amino acid sequence of SEQ ID NO:49, a HCDR2 comprising the amino acid sequence of SEQ ID NO:50, a HCDR3 comprising the amino acid sequence of SEQ ID NO:51, a LCDR1 comprising the amino acid sequence of SEQ ID NO:52, a LCDR2 comprising the amino acid sequence of SEQ ID NO:53, and a LCDR3 comprising the amino acid sequence of SEQ ID NO:54;
a HCDR1 comprising the amino acid sequence of SEQ ID NO:57, a HCDR2 comprising the amino acid sequence of SEQ ID NO:58, a HCDR3 comprising the amino acid sequence of SEQ ID NO:59, a LCDR1 comprising the amino acid sequence of SEQ ID NO:60, a LCDR2 comprising the amino acid sequence of SEQ ID NO:61, and a LCDR3 comprising the amino acid sequence of SEQ ID NO:62;
a HCDR1 comprising the amino acid sequence of SEQ ID NO:84, a HCDR2 comprising the amino acid sequence of SEQ ID NO:85, a HCDR3 comprising the amino acid sequence of SEQ ID NO:86, a LCDR1 comprising the amino acid sequence of SEQ ID NO:87, a LCDR2 comprising the amino acid sequence of SEQ ID NO:88, and a LCDR3 comprising the amino acid sequence of SEQ ID NO:89; or
a HCDR1 comprising the amino acid sequence of SEQ ID NO:84, a HCDR2 comprising the amino acid sequence of SEQ ID NO:92, a HCDR3 comprising the amino acid sequence of SEQ ID NO:86, a LCDR1 comprising the amino acid sequence of SEQ ID NO:87, a LCDR2 comprising the amino acid sequence of SEQ ID NO:93, and a LCDR3 comprising the amino acid sequence of SEQ ID NO:89.

Embodiment 212. The antibody of any of embodiments 201-209, wherein the antibody comprises heavy chain variable domain (V_{H}) and a light chain variable domain (V_{L}) derived from an antibody chosen from dalotuzumab, ganitumab, xentuzumab, AVE1642, figitumumab, dusigitumab, cituxumumab, BIIB022, robatumumab, teprotumumab, and Antibody 2.

Embodiment 213. The antibody of any of embodiments 201-209, comprising:
a heavy chain variable domain comprising the amino acid sequence of SEQ ID NO:7, and a light chain variable domain comprising the amino acid sequence of SEQ ID NO:8;
a heavy chain variable domain comprising the amino acid sequence of SEQ ID NO:15, and a light chain variable domain comprising the amino acid sequence of SEQ ID NO:16;
a heavy chain variable domain comprising the amino acid sequence of SEQ ID NO:23, and a light chain variable domain comprising the amino acid sequence of SEQ ID NO:24;
a heavy chain variable domain comprising the amino acid sequence of SEQ ID NO:31 or 78 or 79, and a light chain variable domain comprising the amino acid sequence of SEQ ID NO:32 or 80 or 81 or 82 or 83;
a heavy chain variable domain comprising the amino acid sequence of SEQ ID NO:39, and a light chain variable domain comprising the amino acid sequence of SEQ ID NO:40;
a heavy chain variable domain comprising the amino acid sequence of SEQ ID NO:47, and a light chain variable domain comprising the amino acid sequence of SEQ ID NO:48;
a heavy chain variable domain comprising the amino acid sequence of SEQ ID NO:55, and a light chain variable domain comprising the amino acid sequence of SEQ ID NO:56;
a heavy chain variable domain comprising the amino acid sequence of SEQ ID NO:63, and a light chain variable domain comprising the amino acid sequence of SEQ ID NO:64; or
a heavy chain variable domain comprising the amino acid sequence of SEQ ID NO:65, and a light chain variable domain comprising the amino acid sequence of SEQ ID NO:66;
a heavy chain variable domain comprising the amino acid sequence of SEQ ID NO:90, and a light chain variable domain comprising the amino acid sequence of SEQ ID NO:91;
a heavy chain variable domain comprising the amino acid sequence of SEQ ID NO:94, or and a light chain variable domain comprising the amino acid sequence of SEQ ID NO:95.

Embodiment 214. The antibody of any of embodiments 201-210, comprising a HCDR1 comprising HCDR1 comprising the amino acid sequence of SEQ ID NO:25 or SEQ ID NO:75, a HCDR2 comprising the amino acid sequence of SEQ ID NO:26 or SEQ ID NO:76 or SEQ ID NO:77, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30.

Embodiment 215. The antibody of any of embodiments 1-9, comprising a HCDR1 comprising the amino acid sequence of SEQ ID NO:25, a HCDR2 comprising the amino acid sequence of SEQ ID NO:76, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30.

Embodiment 216. The antibody of any of embodiments 201-209, comprising
a HCDR1 comprising the amino acid sequence of SEQ ID NO:25, a HCDR2 comprising the amino acid sequence of SEQ ID NO:76, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30;
a HCDR1 comprising the amino acid sequence of SEQ ID NO:25, a HCDR2 comprising the amino acid sequence of SEQ ID NO:77, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30;
a HCDR1 comprising the amino acid sequence of SEQ ID NO:25, a HCDR2 comprising the amino acid sequence of SEQ ID NO:26, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30;
a HCDR1 comprising the amino acid sequence of SEQ ID NO:75, a HCDR2 comprising the amino acid sequence of SEQ ID NO:76, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30;
a HCDR1 comprising the amino acid sequence of SEQ ID NO:75, a HCDR2 comprising the amino acid sequence of SEQ ID NO:77, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30; or
a HCDR1 comprising the amino acid sequence of SEQ ID NO:75, a HCDR2 comprising the amino acid sequence of SEQ ID NO:26, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30.

Embodiment 217. The antibody of any of embodiments 201-209, wherein the antibody comprises a heavy chain variable domain comprising the amino acid sequence of SEQ ID NO:31 or 78 or 79, and a light chain variable domain comprising the amino acid sequence of SEQ ID NO:32 or 80 or 81 or 82 or 83.

Embodiment 218. The antibody of embodiment 217, wherein the antibody comprises a heavy chain variable domain comprising the amino acid sequence of SEQ ID NO:78 and a light chain variable domain comprising the amino acid sequence of SEQ ID NOs:80 or 81 or 82 or 83.

Embodiment 219. The antibody of embodiment 218, wherein the antibody comprises the light chain variable domain comprising the amino acid sequence of SEQ ID NO:80.

Embodiment 220. The antibody of embodiment 219, wherein the antibody comprises the light chain variable domain comprising the amino acid sequence of SEQ ID NO:81.

Embodiment 221. The antibody of embodiment 219, wherein the antibody comprises the light chain variable domain comprising the amino acid sequence of SEQ ID NO:82.

Embodiment 222. The antibody of embodiment 219, wherein the antibody comprises the light chain variable domain comprising the amino acid sequence of SEQ ID NO:83.

Embodiment 223. The antibody of embodiment 218, wherein the antibody comprises a heavy chain variable domain comprising the amino acid sequence of SEQ ID NO:31 and a light chain variable domain comprising the amino acid sequence of SEQ ID NOs:80 or 81 or 82 or 83.

Embodiment 224. The antibody of embodiment 223, wherein the antibody comprises the light chain variable domain comprising the amino acid sequence of SEQ ID NO:80.

Embodiment 225. The antibody of embodiment 223, wherein the antibody comprises the light chain variable domain comprising the amino acid sequence of SEQ ID NO:81.

Embodiment 226. The antibody of embodiment 223, wherein the antibody comprises the light chain variable domain comprising the amino acid sequence of SEQ ID NO:82.

Embodiment 227. The antibody of embodiment 223, wherein the antibody comprises the light chain variable domain comprising the amino acid sequence of SEQ ID NO:83.

Embodiment 228. The antibody of any of embodiments 201-209, comprising a HCDR1 comprising the amino acid sequence of SEQ ID NO:84, a HCDR2 comprising the amino acid sequence of SEQ ID NO:85, a HCDR3 comprising the amino acid sequence of SEQ ID NO:86, a LCDR1 comprising the amino acid sequence of SEQ ID NO:87, a LCDR2 comprising the amino acid sequence of SEQ ID NO:88, and a LCDR3 comprising the amino acid sequence of SEQ ID NO:89.

Embodiment 229. The antibody of any of embodiments 201-209, wherein the antibody comprises a heavy chain variable domain comprising SEQ ID NO:90 and a light chain variable domain comprising SEQ ID NO:91.

Embodiment 230. The antibody of any of embodiments 201-229, wherein the antibody comprises a variant Fc region comprising mutations that substitute a methionine at position 428 with a leucine (Met428Leu) and substitute an asparagine at position 434 with a serine (Asn434Ser), wherein the amino acid substitution numbering is EU as in Kabat.

Embodiment 231. The antibody of any of embodiments 201-229, wherein the antibody comprises a variant Fc region comprising mutations that substitute a first mutation that is a tyrosine at position 252 (Met252Tyr), a second mutation that is a threonine at position 254 (Ser254Thr), and a third mutation that is a glutamic acid at position 256 (Thr256Glu), wherein the amino acid substitution numbering is EU as in Kabat.

Embodiment 232. An antibody which binds to the insulin like growth factor-I receptor (IGF-1R), wherein the antibody comprises a heavy chain variable region that comprises HCDR1, HCDR2, and HCDR3 domains; and a light chain variable region that comprises LCDR1, LCDR2, and LCDR3 domains, comprising:
a HCDR1 comprising HCDR1 comprising the amino acid sequence of SEQ ID NO:25 or SEQ ID NO:75, a HCDR2 comprising the amino acid sequence of SEQ ID NO:26 or SEQ ID NO:76 or SEQ ID NO:77, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30; and either
a variant Fc region comprising mutations that substitute a methionine at position 428 with a leucine (Met428Leu) and substitute an asparagine at position 434 with a serine (Asn434Ser), wherein the amino acid substitution numbering is EU as in Kabat; or
a variant Fc region comprising mutations that substitute a first mutation that is a tyrosine at position 252 (Met252Tyr), a second mutation that is a threonine at position 254 (Ser254Thr), and a third mutation that is a glutamic acid at position 256 (Thr256Glu), wherein the amino acid substitution numbering is EU as in Kabat.

Embodiment 233. An antibody which binds to the insulin like growth factor-I receptor (IGF-1R), wherein the antibody comprises a heavy chain variable region that comprises HCDR1, HCDR2, and HCDR3 domains; and a light chain variable region that comprises LCDR1, LCDR2, and LCDR3 domains, comprising:
a HCDR1 comprising the amino acid sequence of SEQ ID NO:25, a HCDR2 comprising the amino acid sequence of SEQ ID NO:76, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30; and either
a variant Fc region comprising mutations that substitute a methionine at position 428 with a leucine (Met428Leu) and substitute an asparagine at position 434 with a serine (Asn434Ser), wherein the amino acid substitution numbering is EU as in Kabat; or a variant Fc region comprising mutations that substitute a first mutation that is a tyrosine at position 252 (Met252Tyr), a second mutation that is a threonine at position 254 (Ser254Thr), and a third mutation that is a glutamic acid at position 256 (Thr256Glu), wherein the amino acid substitution numbering is EU as in Kabat.

Embodiment 234. An antibody which binds to the insulin like growth factor-I receptor (IGF-1R), wherein the antibody comprises a heavy chain variable region that comprises HCDR1, HCDR2, and HCDR3 domains; and a light chain variable region that comprises LCDR1, LCDR2, and LCDR3 domains, comprising:
a HCDR1 comprising the amino acid sequence of SEQ ID NO:25, a HCDR2 comprising the amino acid sequence of SEQ ID NO:76, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30;
a HCDR1 comprising the amino acid sequence of SEQ ID NO:25, a HCDR2 comprising the amino acid sequence of SEQ ID NO:77, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30;
a HCDR1 comprising the amino acid sequence of SEQ ID NO:25, a HCDR2 comprising the amino acid sequence of SEQ ID NO:26, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30;
a HCDR1 comprising the amino acid sequence of SEQ ID NO:75, a HCDR2 comprising the amino acid sequence of SEQ ID NO:76, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30;
a HCDR1 comprising the amino acid sequence of SEQ ID NO:75, a HCDR2 comprising the amino acid sequence of SEQ ID NO:77, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30; or
a HCDR1 comprising the amino acid sequence of SEQ ID NO:75, a HCDR2 comprising the amino acid sequence of SEQ ID NO:26, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30; and either
a variant Fc region comprising mutations that substitute a methionine at position 428 with a leucine (Met428Leu) and substitute an asparagine at position 434 with a serine (Asn434Ser), wherein the amino acid substitution numbering is EU as in Kabat; or
a variant Fc region comprising mutations that substitute a first mutation that is a tyrosine at position 252 (Met252Tyr), a second mutation that is a threonine at position 254 (Ser254Thr), and a third mutation that is a glutamic acid at position 256 (Thr256Glu), wherein the amino acid substitution numbering is EU as in Kabat.

Embodiment 235. An antibody which binds to the insulin like growth factor-I receptor (IGF-1R), wherein the antibody comprises:
a heavy chain variable domain comprising the amino acid sequence of SEQ ID NO:31 or 78 or 79, and a light chain variable domain comprising the amino acid sequence of SEQ ID NO:32 or 80 or 81 or 82 or 83; and either
a variant Fc region comprising mutations that substitute a methionine at position 428 with a leucine (Met428Leu) and substitute an asparagine at position 434 with a serine (Asn434Ser), wherein the amino acid substitution numbering is EU as in Kabat; or
a variant Fc region comprising mutations that substitute a first mutation that is a tyrosine at position 252 (Met252Tyr), a second mutation that is a threonine at position 254 (Ser254Thr), and a third mutation that is a glutamic acid at position 256 (Thr256Glu), wherein the amino acid substitution numbering is EU as in Kabat.

Embodiment 236. The antibody of embodiment 235, wherein the antibody comprises a heavy chain variable domain comprising the amino acid sequence of SEQ ID NO:78 and a light chain variable domain comprising the amino acid sequence of SEQ ID NOs:80 or 81 or 82 or 83.

Embodiment 237. The antibody of embodiment 236, wherein the antibody comprises the light chain variable domain comprising the amino acid sequence of SEQ ID NO:80.

Embodiment 238. The antibody of embodiment 236, wherein the antibody comprises the light chain variable domain comprising the amino acid sequence of SEQ ID NO:81.

Embodiment 239. The antibody of embodiment 236, wherein the antibody comprises the light chain variable domain comprising the amino acid sequence of SEQ ID NO:82.

Embodiment 240. The antibody of embodiment 236, wherein the antibody comprises the light chain variable domain comprising the amino acid sequence of SEQ ID NO:83.

Embodiment 241. The antibody of embodiment 235, wherein the antibody comprises a heavy chain variable domain comprising the amino acid sequence of SEQ ID NO:31 and a light chain variable domain comprising the amino acid sequence of SEQ ID NOs:80 or 81 or 82 or 83.

Embodiment 242. The antibody of embodiment 241, wherein the antibody comprises the light chain variable domain comprising the amino acid sequence of SEQ ID NO:80.

Embodiment 243. The antibody of embodiment 241, wherein the antibody comprises the light chain variable domain comprising the amino acid sequence of SEQ ID NO:81.

Embodiment 244. The antibody of embodiment 241, wherein the antibody comprises the light chain variable domain comprising the amino acid sequence of SEQ ID NO:82.

Embodiment 245. The antibody of embodiment 241, wherein the antibody comprises the light chain variable domain comprising the amino acid sequence of SEQ ID NO:83.

Embodiment 246. An antibody which binds to the insulin like growth factor-I receptor (IGF-1R), wherein the antibody comprises a heavy chain variable region that comprises HCDR1, HCDR2, and HCDR3 domains; and a light chain variable region that comprises LCDR1, LCDR2, and LCDR3 domains, comprising:
a HCDR1 comprising the amino acid sequence of SEQ ID NO:84, a HCDR2 comprising the amino acid sequence of SEQ ID NO:85, a HCDR3 comprising the amino acid sequence of SEQ ID NO:86, a LCDR1 comprising the amino acid sequence of SEQ ID NO:87, a LCDR2 comprising the amino acid sequence of SEQ ID NO:88, and a LCDR3 comprising the amino acid sequence of SEQ ID NO:89; and either
a variant Fc region comprising mutations that substitute a methionine at position 428 with a leucine (Met428Leu) and substitute an asparagine at position 434 with a serine (Asn434Ser), wherein the amino acid substitution numbering is EU as in Kabat; or
a variant Fc region comprising mutations that substitute a first mutation that is a tyrosine at position 252 (Met252Tyr), a second mutation that is a threonine at position 254 (Ser254Thr), and a third mutation that is a glutamic acid at position 256 (Thr256Glu), wherein the amino acid substitution numbering is EU as in Kabat.

Embodiment 247. An antibody which binds to the insulin like growth factor-I receptor (IGF-1R), wherein the antibody comprises:
a heavy chain variable domain comprising SEQ ID NO:7 and a light chain variable domain comprising SEQ ID NO:8; and either
a variant Fc region comprising mutations that substitute a methionine at position 428 with a leucine (Met428Leu) and substitute an asparagine at position 434 with a serine (Asn434Ser), wherein the amino acid substitution numbering is EU as in Kabat; or
a variant Fc region comprising mutations that substitute a first mutation that is a tyrosine at position 252 (Met252Tyr), a second mutation that is a threonine at position 254 (Ser254Thr), and a third mutation that is a glutamic acid at position 256 (Thr256Glu), wherein the amino acid substitution numbering is EU as in Kabat.

Embodiment 248. The antibody of any of embodiments 232-247, wherein the antibody comprises a variant Fc region comprising mutations that substitute a methionine at position 428 with a leucine (Met428Leu) and substitute an asparagine at position 434 with a serine (Asn434Ser), wherein the amino acid substitution numbering is EU as in Kabat.

Embodiment 249. The antibody of any of embodiments 232-247, wherein the antibody comprises a variant Fc region comprising mutations that substitute a first mutation that is a tyrosine at position 252 (Met252Tyr), a second mutation that is a threonine at position 254 (Ser254Thr), and a third mutation that is a glutamic acid at position 256 (Thr256Glu), wherein the amino acid substitution numbering is EU as in Kabat.

Embodiment 250. A nucleotide sequence encoding the polypeptide sequence of the antibody of any of embodiments 201-249.

Embodiment 251. An expression vector comprising the nucleotide sequence of embodiment 250.

Embodiment 252. A Chinese hamster ovary (CHO) cell line expressing the vector of embodiment 251.

Embodiment 253. A pharmaceutical composition comprising a therapeutically effective amount of the antibody as recited in any of embodiments 201-249, and a pharmaceutically acceptable carrier.

Embodiment 254. The pharmaceutical composition of embodiment 253, wherein the therapeutically effective amount comprises a dosage of 1-10 mg/kg.

Embodiment 255. The pharmaceutical composition of embodiment 254, wherein the therapeutically effective amount comprises a dosage of 1-5 mg/kg.

Embodiment 256. The pharmaceutical composition of embodiment 255, wherein the therapeutically effective amount comprises a dosage of about 2 mg/kg, about 3 mg/kg, about 4 mg/kg, or about 5 mg/kg.

Embodiment 257. The pharmaceutical composition of any of embodiments 253-256, wherein the therapeutically effective amount is formulated for administration every 1, 2, 3, 4, or 5 weeks (i.e., QW, Q2W, Q3W, Q4W, or Q5W).

Embodiment 258. The pharmaceutical composition of any of embodiments 253-257, wherein the pharmaceutically acceptable carrier is suitable for intravenous (IV) or subcutaneous (SC) administration.

Embodiment 259. The pharmaceutical composition of embodiment 253, comprising a therapeutically effective amount of the antibody as recited in any of embodiments 201-249, wherein the therapeutically effective amount comprises a dosage of:
1-60 mg/kg or 75-4500 mg; or
0.6-40 mg/kg or 45-3000 mg; or
0.3-20 mg/kg or 22-1500 mg.

Embodiment 260. The pharmaceutical composition of embodiment 259, formulated for IV administration.

Embodiment 261. The pharmaceutical composition of embodiment 260, formulated for dosing every 4, 3, 2, or 1 weeks.

Embodiment 262. The pharmaceutical composition of embodiment 253, comprising a therapeutically effective amount of the antibody as recited in any of embodiments 201-249, wherein the therapeutically effective amount comprises a dosage of:
1-30 mg/kg or 75-2250 mg;
0.6-20 mg/kg or 1500 mg;
0.3-10 mg/kg or 750 mg.

Embodiment 263. The pharmaceutical composition of embodiment 253, comprising a therapeutically effective amount of the antibody as recited in any of embodiments 201-249, wherein the therapeutically effective amount comprises a dosage of:
1-20 mg/kg or 75-1500 mg;
0.6-13.5 mg/kg or 1000 mg;
0.3-7 mg/kg or 500 mg.

Embodiment 264. The pharmaceutical composition of any of embodiments 262-263, formulated for subcutaneous administration.

Embodiment 265. The pharmaceutical composition of embodiment 264, formulated for dosing every 4, 3, 2, or 1 weeks.

Embodiment 266. An autoinjector comprising the pharmaceutical formulation as recited in embodiment 264.

Embodiment 267. A method of treating thyroid eye disease (TED) in a subject with TED, comprising administering to the subject a therapeutically effective amount of the antibody of any of embodiments 201-249 or the pharmaceutical composition of any of embodiments 253-265.

Embodiment 268. A method of reducing proptosis in a subject with thyroid eye disease (TED), comprising administering to the subject a therapeutically effective amount of the antibody of any of embodiments 201-249 or the pharmaceutical composition of any of embodiments 253-265.

Embodiment 269. The method of embodiment 268, wherein proptosis is reduced by at least 2 mm.

Embodiment 270. The method of embodiment 269, wherein proptosis is reduced by at least 3 mm.

Embodiment 271. The method of embodiment 270, wherein proptosis is reduced by at least 4 mm.

Embodiment 272. The method of embodiment 268, wherein the method additionally comprises reducing the clinical activity score (CAS) in the subject with TED.

Embodiment 273. The method of embodiment 272, wherein proptosis is reduced by at least 2 mm and CAS is reduced by at least 2 points.

Embodiment 274. The method of embodiment 273, wherein CAS is reduced by at least 3 points.

Embodiment 275. The method of embodiment 274, wherein proptosis is reduced by at least 3 mm and CAS is reduced by at least 3 points.

Embodiment 276. A method of treating or reducing the severity of diplopia in a subject with thyroid eye disease (TED), comprising administering to the subject a therapeutically effective amount of the antibody of any of embodiments 201-249 or the pharmaceutical composition of any of embodiments 253-265.

Embodiment 277. The method of embodiment 276, wherein the diplopia is constant diplopia.

Embodiment 278. The method of embodiment 276, wherein the diplopia is intermittent diplopia.

Embodiment 279. The method of embodiment 276, wherein the diplopia is inconstant diplopia.

Embodiment 280. The method of embodiment 276, wherein the improvement in or reduction in severity of diplopia is sustained at least 20 weeks after discontinuation of inhibitor administration.

Embodiment 281. The method of embodiment 280, wherein the improvement in or reduction in severity of diplopia is sustained at least 50 weeks after discontinuation of inhibitor administration.

Embodiment 282. A method of reducing Clinical Activity Score (CAS) of thyroid eye disease (TED) in a subject with TED, comprising administering to a subject in need thereof a therapeutically effective amount of the antibody of any of embodiments 201-249 or the pharmaceutical composition of any of embodiments 253-265.

Embodiment 283. The method of embodiment 282, wherein CAS is reduced by at least 2 points.

Embodiment 284. The method of embodiment 283, wherein CAS is reduced by at least 3 points.

Embodiment 285. A method of:
reducing insulin like growth factor-I receptor (IGF-1R) signaling;
inhibiting thyroid stimulating hormone receptor (TSHR)/IGF-1R crosstalk (i.e., formation of a TSHR/IGF-1R signalosome); and/or
reducing hyaluronan (HA) secretion in orbital fibroblasts;
in a subject with TED, comprising administering to a subject in need thereof a therapeutically effective amount of the antibody of any of claims 1-49 or the pharmaceutical composition of any of claims 53-65,
wherein the antibody persists for an extended period of time in vivo (i.e., has a longer half-life) compared to an antibody that does not comprise the M428L/N434S or M252Y/S254T/T256E substitutions; and/or
wherein the antibody or pharmaceutical composition is administered less frequently, or in a lower amount per dose, compared to an antibody that does not comprise the M428L/N434S or M252Y/S254T/T256E substitutions.

Embodiment 286. The method of any of embodiments 267-285, wherein the therapeutically effective amount comprises a dosage of 1-10 mg/kg.

Embodiment 287. The method of embodiment 286, wherein the therapeutically effective amount comprises a dosage of 1-5 mg/kg.

Embodiment 288. The method of embodiment 287, wherein the therapeutically effective amount comprises a dosage of about 2 mg/kg, about 3 mg/kg, about 4 mg/kg, or about 5 mg/kg.

Embodiment 289. The method of any of embodiments 267-288, wherein the therapeutically effective amount is administered every 1, 2, 3, 4, or 5 weeks (i.e., QW, Q2W, Q3W, Q4W, or Q5W).

Embodiment 290. The method of any of embodiments 267-289, wherein the therapeutically effective amount is administered intravenously (IV) or subcutaneously (SC).

Embodiment 291. The method of any of embodiments 267-284, comprising a therapeutically effective amount of the antibody as recited in any of embodiments 201-249, wherein the therapeutically effective amount comprises a dosage of
1-5 mg/kg or 75-375 mg IV Q3W; or
0.6-4 mg/kg or 45-300 mg IV Q2W; or
0.3-3 mg/kg or 22-225 mg IV QW.

Embodiment 292. The method of embodiment 291, formulated for IV administration.

Embodiment 293. The method of embodiment 292, formulated for dosing every 4, 3, 2, or 1 weeks.

Embodiment 294. The method of any of embodiments 267-285, comprising a therapeutically effective amount of the antibody as recited in any of embodiments 201-249, wherein the therapeutically effective amount comprises a dosage of:
1-30 mg/kg or 75-2250 mg;
0.6-20 mg/kg or 1500 mg;
0.3-10 mg/kg or 750 mg.

Embodiment 295. The method of any of embodiments 267-285, comprising a therapeutically effective amount of the antibody as recited in any of embodiments 201-249, wherein the therapeutically effective amount comprises a dosage of:
1-20 mg/kg or 75-1500 mg;
0.6-13.5 mg/kg or 1000 mg;
0.3-7 mg/kg or 500 mg.

Embodiment 296. The method of any of embodiments 294-295, formulated for subcutaneous administration.

Embodiment 297. The method of embodiment 296, formulated for dosing every 4, 3, 2, or 1 weeks.

Embodiment 298. The method of embodiment 296, wherein the subcutaneous administration is done using an autoinjector.

Embodiment 299. The use of the antibody of any of embodiments 201-249, or the pharmaceutical composition of any of embodiments 253-265 or the autoinjector of embodiment 266, for the treatment of thyroid eye disease (TED) in a subject with TED, reduction of proptosis in a subject with TED, reduction of the severity of diplopia in a subject with TED, or reduction of CAS in a subject with TED, as recited in any of embodiments 267-298.

Embodiment 300. The use of the antibody of any of embodiments 201-249, or the pharmaceutical composition of any of embodiments 253-265 or the autoinjector of embodiment 266, in the manufacture of a medicament for the treatment of thyroid eye disease (TED) in a subject with TED, reduction of proptosis in a subject with TED, reduction of the severity of diplopia in a subject with TED, or reduction of CAS in a subject with TED, as recited in any of embodiments 267-298.

Also provided herein are the following embodiments.

Provided herein are methods of treating or reducing the severity of thyroid eye disease (TED), comprising administering to the subject an effective amount of an insulin like growth factor-I receptor (IGF 1R) inhibitor.

In some embodiments, said IGF-1R inhibitor is an antibody.

In some embodiments, said antibody IGF-1R inhibitor is chosen from ganitumab, figitumumab, dusigitumab, cixutumumab, dalotuzumab, robatumumab, AVE1642, BIIB022, and xentuzumab.

In some embodiments, said IGF-1R inhibitor is a small molecule.

In some embodiments, said small molecule IGF-1R inhibitor is chosen from linsitinib, picropodophyllin, BMS-754807, BMS-536924, BMS-554417, GSK1838705A, NVP-AEW541, GTx-134, and AG1024.

Also provided herein is a method of reducing proptosis (e.g., by at least 2 mm) in a subject with thyroid-associated ophthalmopathy thyroid eye disease (TED) comprising administering to the subject an effective amount of an IGF-1R inhibitor.

Also provided herein is a method of reducing proptosis (e.g., by at least 2 mm) and reducing the clinical activity score (CAS) in a subject with thyroid-associated ophthalmopathy thyroid eye disease (TED) comprising administering to the subject an effective amount of an IGF-1R inhibitor.

Also provided herein is a method of treating or reducing the severity of thyroid eye disease (TED) comprising administering to a subject in need thereof, an effective amount of an IGF-1R inhibitor, and wherein the IGF-1R inhibitor (i) reduces proptosis by at least 2 mm; and (ii) reduces the CAS in the subject by at least 2 points (on the 7-point version of the scale—as described below).

Also provided herein is a method of reducing proptosis by at least 4 mm in a subject with thyroid eye disease (TED) comprising administering to the subject an effective amount of an IGF-1R inhibitor.

Also provided herein is a method of treating or reducing the severity of thyroid eye disease (TED) comprising administering to a subject in need thereof an effective amount of an IGF-1R inhibitor, and wherein the IGF-1R inhibitor reduces proptosis by at least 4 mm.

Also provided herein is a method of treating or reducing the severity of diplopia in a subject with thyroid eye disease (TED), comprising administering to the subject an effective amount of an IGF-1R inhibitor.

Also provided herein is a method of reducing the severity of thyroid eye disease (TED) comprising administering to a subject in need thereof an effective amount of a pharmaceutical composition comprising an IGF-1R inhibitor, and a pharmaceutically acceptable excipient or diluent or carrier.

Accordingly, provided herein is a method of reducing proptosis by at least 2 mm in a subject with TED (TAO or GO). The method comprises administering to the subject an effective amount of an IGF-1R inhibitor.

Also provided herein is a method of reducing proptosis by at least 2 mm and reducing the clinical activity score (CAS) in a subject with TED (TAO or GO), comprising administering to the subject an effective amount of an IGF-1R inhibitor.

Also provided herein is a method of treating or reducing the severity of TED (TAO or GO). The method comprises administering to a subject in need thereof, an effective amount of an IGF-1R inhibitor, and wherein the IGF-1R inhibitor (i) reduces proptosis by at least 2 mm; and (ii) reduces the CAS in the subject by at least 2 points (on the 7-point version of the scale).

In some embodiments, the reduction in proptosis or exophthalmos could be greater than 2 mm, for example, 2.2 mm, 2.4 mm, 2.5 mm, 2.6 mm. 2.8 mm, 3 mm, 3.2 mm, 3.4 mm, 3.5 mm, 3.6 mm, 3.8 mm, 4 mm, 4.1 mm, 4.2 mm, 4.3 mm, 4.4 mm, 4.5 mm, 4.6 mm, 4.7 mm, 4.8 mm, 4.9 mm, 5 mm or more than 5 mm.

In some embodiments, the reduction in CAS is by 2 points or more, for example, by 3, 4, 5, 6, or 7 points. In one embodiment, the reduction in CAS is by 2 or more points. In another embodiment, it is by 3 or more points. In yet another embodiment, the reduction in CAS is by 4 or more points.

Also provided herein is a method of reducing proptosis by at least 4 mm in a subject with TED (TAO or GO). The method comprises administering to the subject an effective amount of an IGF-1R inhibitor.

Also provided herein is a method of treating or reducing the severity of TED. The method comprises administering to a subject in need thereof an effective amount of an IGF-1R inhibitor, and wherein the IGF-1R inhibitor reduces proptosis or exophthalmos by at least 3 mm. Also provided herein is a method of treating or reducing the severity of TED. The method comprises administering to a subject in need thereof, an effective amount of an IGF-1R inhibitor, and wherein the IGF-1R inhibitor reduces proptosis or exophthalmos by at least 4 mm.

Also provided herein is a method of treating or reducing the severity of diplopia associated with TED (in a subject with TED and diplopia), comprising administering to the subject, an effective amount of an IGF-1R inhibitor.

Also provided herein is a method of treating or reducing the severity of diplopia in a subject with thyroid eye disease (TED), comprising administering to the subject an effective amount of an IGF-1R inhibitor.

When TED is severe, this active autoimmune disease, characterized by orbital tissue remodeling from activation of TSH and IGF-1 receptors, results in excess extracellular matrix and proptosis/diplopia, a major quality of life (QoL) issue for TED patients.

Also provided herein is a method of treating or reducing the severity of constant diplopia (CD) in a subject with thyroid eye disease (TED), comprising administering to the subject, an effective amount of an IGF-1R inhibitor. Also provided herein is a method of treatment of diplopia comprising administering to the subject, an effective amount of an IGF-1R inhibitor, that results in improved diplopia relative to placebo.

It should be noted that not all subjects respond to administration of the IGF-1R inhibitor in the same manner. When administered to a population of patients, about 20%, 25%, 30%, 35%, 40%, 45%, 50%, 55%, 60%, 65%, 70%, 75%, 80%, 85%, 90%, 95% or 100% of the patients may respond with a reduction in proptosis or exophthalmos by at least 2 mm and a reduction in the CAS by at least 2 points. In some embodiments, the response is seen in at least 20%, or at least 25%, or at least 30%, or at least 35%, or at least 40%, or at least 45%, or at least 50%, or at least 55%, or at least 60%, or at least 65%, or at least 70%, or at least 80% of the patients.

In some embodiments, the IGF-1R inhibitor reduces proptosis by at least 3 mm in at least 25%, 30%, 35%, 40%, 45% 50%, 55%, 60%, 65%, 70%, 75%, 80%, or 85% of the subjects. In some embodiments, the IGF-1R inhibitor reduces proptosis by at least 3.5 mm in at least 25%, 30%, 35%, 40%, 45% 50%, 55%, 60%, 65%, 70%, 75%, 80%, or 85% of the subjects. In some embodiments, the IGF-1R inhibitor reduces proptosis by at least 4 mm in at least 25%, 30%, 35%, 40%, 45% 50%, 55%, 60%, 65%, 70%, 75%, 80%, or 85% of the subjects. In some embodiments, the IGF-1R inhibitor reduces proptosis by at least 4 mm in about 40% of the subjects.

Also provided herein is a method of reducing proptosis in an eye in a subject with thyroid eye disease (TED), thyroid-associated ophthalmopathy (TAO), or Graves' ophthalmopathy (GO) who has previously undergone prior treatment with an IGF-1R inhibitor and either did not respond to said prior treatment or responded to said prior treatment and later relapsed, comprising administering to said subject an effective amount of the IGF-1R inhibitor.

Also provided herein is a method of reducing proptosis by at least 2 mm in an eye without a deterioration of 2 mm or more in the other (or fellow eye) in a subject with TED comprising administering to said subject an effective amount of an IGF-1R inhibitor. The subject is one who has undergone prior treatment with said IGF-1R inhibitor, and either did not respond to said prior treatment or relapsed after said prior treatment.

In some embodiments, the reduction in proptosis or exophthalmos could be greater than 2 mm, for example, 2.2 mm, 2.4 mm, 2.5 mm, 2.6 mm. 2.8 mm, 3 mm, 3.2 mm, 3.4 mm, 3.5 mm, 3.6 mm, 3.8 mm, 4 mm, 4.1 mm, 4.2 mm, 4.3 mm, 4.4 mm, 4.5 mm, 4.6 mm, 4.7 mm, 4.8 mm, 4.9 mm, 5 mm or more than 5 mm.

Also provided herein is a method of reducing Clinical Activity Score (CAS) of thyroid eye disease (TED) in a subject who has undergone prior treatment with an IGF-1R inhibitor, and either did not respond to said prior treatment or relapsed after said prior treatment, comprising administering to a subject in need thereof an effective amount of an IGF-1R inhibitor.

In some embodiments, CAS is reduced in said subject to either one (1) or zero (0) (on the 7-point version of the CAS scale—as described below).

In some embodiments, the reduction in CAS is by 2 points or more, for example, by 3, 4, 5, 6, or 7 points. In one embodiment, the reduction in CAS is by 2 or more points. In another embodiment, it is by 3 or more points. In yet another embodiment, the reduction in CAS is by 4 or more points. In yet another embodiment, the reduction in CAS is by 5 or more points.

In one embodiment, as a result of the treatment, the CAS is reduced to one (1). In another embodiment, as a result of the treatment, the CAS is reduced to zero (0).

Also provided herein is a method of treating or reducing the severity of thyroid eye disease (TED) comprising administering to a subject who has undergone prior treatment with an IGF-1R inhibitor, and either did not respond to said prior treatment or responded to said prior treatment and later relapsed, IGF-1R inhibitor.

Also provided herein is a method of treating or reducing the severity of thyroid eye disease (TED) in a subject who has undergone prior treatment with an IGF-1R inhibitor and either did not respond to said prior treatment or relapsed after said prior treatment comprising administering to a subject in need thereof an effective amount of an IGF-1R inhibitor, and wherein said IGF-1R inhibitor (i) reduces proptosis by at least 2 mm in an eye; (ii) is not accompanied by a deterioration of 2 mm or more in the other (or fellow eye); and (iii) reduces the CAS in said subject to either one (1) or zero (0) (on the 7-point version of the scale—as described below.

Also provided herein is a method of treating or reducing the severity of thyroid eye disease (TED; TAO or GO) comprising administering to a subject in need thereof an effective amount of an IGF-1R inhibitor, wherein said antibody reduces proptosis by at least 2 mm as well as reduces the CAS to either one (1) or zero (0). As stated above, the subject is one who has undergone prior treatment with said IGF-1R inhibitor, and either did not respond to said prior treatment or relapsed after said prior treatment.

Also provided herein is a method of treating or reducing the severity of thyroid eye disease (TED; TAO or GO) in a subject with TED who has previously undergone prior treatment with an IGF-1R inhibitor and either did not respond to said prior treatment or responded to said prior treatment and later relapsed, comprising administering to a subject in need thereof an effective amount of a pharmaceutical composition comprising an IGF-1R inhibitor and a pharmaceutically acceptable excipient or diluent or carrier.

Also provided herein is a method of reducing proptosis in an eye in a subject with thyroid eye disease (TED; TAO or GO) who has previously undergone prior treatment with an IGF-1R inhibitor and either did not respond to said prior treatment or responded to said prior treatment and later relapsed, comprising administering to said subject an effective amount of the IGF-1R inhibitor.

Also provided herein is a method of treating or reducing the severity of thyroid eye disease (TED; TAO or GO) comprising administering to a subject who has undergone prior treatment with an IGF-1R inhibitor, and either did not respond to said prior treatment or responded to said prior treatment and later relapsed, the IGF-1R inhibitor.

Also provided herein is a method of improving the quality of life in a subject with thyroid eye disease (TED; TAO or GO) who has undergone prior treatment with an IGF-1R inhibitor, and either did not respond to said prior treatment or responded to said prior treatment and later relapsed, comprising administering to the subject an effective amount of an IGF-1R inhibitor.

Also provided herein is a method of treating or reducing diplopia or the severity of diplopia in a subject with thyroid eye disease (TED; TAO or GO) who has undergone prior treatment with an IGF-1R inhibitor, and either did not respond to said prior treatment or responded to said prior treatment and later relapsed, comprising administering to the subject an effective amount of an IGF-1R inhibitor.

In some embodiments, the diplopia is constant diplopia. In some embodiments, the diplopia is inconstant diplopia. In some embodiments, the diplopia is intermittent diplopia.

In some embodiments, the improvement in or reduction in severity of diplopia is sustained at least 20, 30, 40, or 50 weeks after discontinuation of IGF-1R inhibitor administration. In some embodiments, the improvement in or reduction in severity of diplopia is sustained 20-30, 30-40, 40-50, or 50-60 weeks after discontinuation of IGF-1R inhibitor administration. In some embodiments, the improvement in or reduction in severity of diplopia is sustained at least 20 weeks after discontinuation of IGF-1R inhibitor administration. In some embodiments, the improvement in or reduction in severity of diplopia is sustained at least 50 weeks after discontinuation of IGF-1R inhibitor administration.

Also provided herein is a method of treating or reducing the severity of constant diplopia (CD) in a subject with thyroid eye disease (TED; TAO or GO) who has undergone prior treatment with an IGF-1R inhibitor, and either did not respond to said prior treatment or responded to said prior treatment and later relapsed, comprising administering to the subject an effective amount of an IGF-1R inhibitor. In some embodiments, the treatment with the IGF-1R inhibitor improves the CD QoL in patients with severe TED.

Also provided herein is a method of treating or reducing the severity of diplopia in a subject with thyroid eye disease (TED; TAO or GO) who has undergone prior treatment with an IGF-1R inhibitor, and either did not respond to said prior treatment or responded to said prior treatment and later relapsed, comprising administering to the subject an effective amount of an IGF-1R inhibitor, that results in improved diplopia relative to placebo which is sustained out to 51 weeks after drug discontinuation.

The IGF-1R inhibitor can be administered in a single dose or in multiple doses. In one embodiment, the IGF-1R inhibitor is administered to the subject in a single dose. In another embodiment, the IGF-1R inhibitor is administered to the subject in multiple doses, spread out over the course of a few days, weeks or months. In some embodiments the IGF-1R inhibitor is administered every week or every 2 weeks or every 3 weeks or every 4 weeks or every 5 weeks or every 6 weeks or every 7 weeks or every 8 weeks or every month or every 2 months or every 3 months.

In some embodiments the IGF-1R inhibitor is administered in multiple doses and the dosage is the same each time. In some embodiments the IGF-1R inhibitor is administered in multiple doses and the dosage at the time of first administration is different (could be higher or lower) from those at subsequent times. In some embodiments the IGF-1R inhibitor is administered in multiple doses and the dosage is adjusted at each administration based on the subject's response to the therapy.

The dosage may further vary between patients, based on different factors such as the age, gender, race, and body weight of each patient. In one embodiment, the dosage varies by body weight of the patient. The dosage could range from about 1 mg of the IGF-1R inhibitor per kilogram of body weight to about 100 mg of the IGF-1R inhibitor per kilogram of body weight. The dosage, could for example, be 1 mg, 2 mg, 3 mg, 5 mg, 7 mg, 10 mg, 12 mg, 15 mg, 17 mg, 20 mg, 22 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg or 100 mg, of the IGF-1R inhibitor per kilogram of body weight.

In some embodiments, the dosage is about 1 mg/kg to about 5 mg/kg of the IGF-1R inhibitor. In some embodiments, the dosage is about 5 mg/kg to about 10 mg/kg of the IGF-1R inhibitor. In some embodiments, the dosage is about 10 mg/kg to about 15 mg/kg of the IGF-1R inhibitor. In some embodiments, the dosage is about 15 mg/kg to about 20 mg/kg of the IGF-1R inhibitor.

In some embodiments where the IGF-1R inhibitor is administered in multiple doses and the dosage at the time of first administration is different from those at subsequent times, the dosage at the time of first administration is about 1 mg/kg to about 5 mg/kg of the IGF-1R inhibitor; or about 5 mg/kg to about 10 mg/kg of the IGF-1R inhibitor; or about 10 mg/kg to about 15 mg/kg of the IGF-1R inhibitor; or about 15 mg/kg to about 20 mg/kg of the IGF-1R inhibitor; or about 20 mg/kg to about 25 mg/kg of the IGF-1R inhibitor. The subsequent dose(s) could be higher or lower than the first dose. In some embodiments, the subsequent dose is about 1 mg/kg to about 5 mg/kg of the IGF-1R inhibitor; or about 5 mg/kg to about 10 mg/kg of the IGF-1R inhibitor; or about 10 mg/kg to about 15 mg/kg of IGF-1R inhibitor; or about 15 mg/kg to about 20 mg/kg of the IGF-1R inhibitor; or about 20 mg/kg to about 25 mg/kg of the IGF-1R inhibitor.

The duration of the treatment would depend on the subject's response to the therapy and can range from about one month or 4 weeks to about 2 years or 100 weeks. In different embodiments, the treatment may be provided over a total duration of about 1 month, 2 months, 3 months, 4 months, 5 months, 6 months, 7 months, 8 months, 9 months, 10 months, 11 months, 1 year, 14 months, 16 months, 18 months, 20 months, 22 months or 2 years. In other embodiments, the treatment may be provided over a total duration of 4, 6, 8, 10, 12, 14, 16, 20, 22, 24, 26, 28, 30, 32, 34, 36, 38, 40, 42, 44, 46, 48, 50, 52 weeks, or extended to 56, 64, 72, 80, 88, 96 or 104 weeks.

The IGF-1R inhibitor may be administered by any suitable route including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes. Hyposprays may also be used to administer the pharmaceutical compositions disclosed herein. Solid forms suitable for solution in, or suspension in, liquid vehicles prior to injection may also be used.

Also provided are embodiments wherein any embodiment above may be combined with any one or more of these embodiments, provided the combination is not mutually exclusive. As used herein, two embodiments are “mutually exclusive” when one is defined to be something which is different than the other.

### Definitions

To facilitate understanding of the disclosure, a number of terms and abbreviations as used herein are defined below as follows:

As used herein, the term “antibody” encompasses the various forms of antibodies including but not being limited to whole antibodies, monoclonal antibodies, antibody fragments, human antibodies, humanized antibodies, chimeric antibodies and genetically engineered antibodies as long as the characteristic properties such as specificity and IGF-IR inhibitory are retained.

As used herein, the terms “antigen binding fragment,” “fragment,” and “antibody fragment” are used interchangeably to refer to any fragment that comprises a portion of a full length antibody, generally at least the antigen binding portion or the variable region thereof. Examples of antibody fragments include, but are not limited to, diabodies, single-chain antibody molecules, multispecific antibodies, Fab, Fab′, F(ab′)₂, Fv or scFv. Further, the term “antibody” as used herein includes both antibodies and antigen binding fragments thereof. In addition, antibody fragments comprise single chain polypeptides having the characteristics of a VH chain, namely being able to assemble together with a VL chain or of a VL chain binding to IGF-IR, namely being able to assemble together with a VH chain to a functional antigen binding pocket and thereby providing the property of inhibiting the binding of IGF-I and IGF-II to IGF-IR.

The terms “monoclonal antibody” or “monoclonal antibody composition,” as used herein refer to a preparation of antibody molecules of a single amino acid composition. Accordingly, the term “human monoclonal antibody” refers to antibodies displaying a single binding specificity which have variable and constant regions derived from human germline immunoglobulin sequences. In one embodiment, the human monoclonal antibodies are produced by a hybridoma which includes a B cell obtained from a transgenic non-human animal, e.g., a transgenic mouse, having a genome comprising a human heavy chain transgene and a light human chain transgene fused to an immortalized cell.

The term “human antibody” as used herein, is intended to include antibodies having variable and constant regions derived from human germline immunoglobulin sequences. The term “humanized antibody” as used herein refers to antibodies in which the framework or “complementarity determining regions” (CDR) have been modified to comprise the CDR of an immunoglobulin of different specificity as compared to that of the parent immunoglobulin. In a preferred embodiment, a murine CDR is grafted into the framework region of a human antibody to prepare the “humanized antibody.”

The term “recombinant human antibody,” as used herein, is intended to include all human antibodies that are prepared, expressed, created or isolated by recombinant means, such as antibodies isolated from a host cell such as an SP2-0, NS0 or CHO cell or from an animal (e.g., a mouse) that is transgenic for human immunoglobulin genes or antibodies expressed using a recombinant expression vector transfected into a host cell. Such recombinant human antibodies have variable and constant regions derived from human germline immunoglobulin sequences in a rearranged form.

The term “variable region” (variable region of a light chain (VL), variable region of a heavy chain (VH)) as used herein denotes each of the pair of light and heavy chains which is involved directly in binding the antibody to the antigen. The domains of variable human light and heavy chains have the same general structure and each domain comprises four framework (FR) regions whose sequences are widely conserved, connected by three “hypervariable regions” (or complementarity determining regions, CDRs). The framework regions adopt a β-sheet conformation and the CDRs may form loops connecting the β-sheet structure. The CDRs in each chain are held in their three-dimensional structure by the framework regions and form together with the CDRs from the other chain the antigen binding site. The antibody heavy and light chain CDR3 regions play an important role in the binding specificity/affinity of antibodies.

The terms “complementarity determining region,” “CDR,” “hypervariable region,” or “antigen-binding portion of an antibody” are used interchangeably herein and refer to the amino acid residues of an antibody which are responsible for antigen-binding. The hypervariable region comprises amino acid residues from the complementarity determining regions or CDRs. “Framework” or “FR” regions are those variable domain regions other than the hypervariable region residues as herein defined. Therefore, the light and heavy chains of an antibody comprise from N- to C-terminus the domains FR1, CDR1, FR2, CDR2, FR3, CDR3, and FR4. Especially, CDR3 of the heavy chain is the region which contributes most to antigen binding. CDR and FR regions are determined according to the standard definition of Kabat et al., Sequences of Proteins of Immunological Interest, 5th Ed. Public Health Service, National Institutes of Health, Bethesda, MD. (1991)) and/or those residues from a “hypervariable loop.”

The terms “binding to IGF-IR” or “specific binding to IGF-IR” are used interchangeably herein and mean the binding of the antibody to IGF-IR in an in vitro assay, preferably in a binding assay in which the antibody is bound to a surface and binding of IGF-IR is measured by Surface Plasmon Resonance (SPR). Binding means a binding affinity (K_{D}) of 10⁻⁸ M or less, preferably 10⁻¹³ to 10⁻⁹ M. Binding to IGF-IR can be investigated by a BIAcore assay (Pharmacia Biosensor AB, Uppsala, Sweden). The affinity of the binding is defined by the terms ka (rate constant for the association of the antibody from the antibody/antigen complex), kd (dissociation constant), and K_{D} (kd/ka). The antibodies used in the methods disclose herein typically show a K_{D} of about 10⁻⁹ M or less.

The antibodies, or antigen binding fragments thereof, used in the methods disclosed herein inhibit the binding of IGF-I and IGF-II to IGF-IR. The inhibition is measured as IC₅₀ in an assay for binding of IGF-I/IGF-II to IGF-IR on cells. Such an assay is known to one of skill in the art and is described, for example, U.S. Pat. No. 7,579,157, which is incorporated herein in its entirety. The IC₅₀ values of the antibodies used in the methods disclosed herein for the binding of IGF-I and IGF-II to IGF-IR typically are no more than 2 nM. IC₅₀ values are measured as average or median values of at least three independent measurements. Single IC₅₀ values may be excluded from the scope.

The term “inhibiting the binding of IGF-I and IGF-II to IGF-IR” as used herein refers to inhibiting the binding of I¹²⁵-labeled IGF-I or IGF-II to IGF-IR presented on the surface of cells in an in vitro assay. Inhibiting means an IC₅₀ value of 2 nM or lower.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a subject or patient is intended to include prevention, prophylaxis, attenuation, amelioration and therapy. Treatment may also include prevention of disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The terms “subject” and “patient” are used interchangeably herein to mean all mammals including humans. Examples of subjects include, but are not limited to, humans, monkeys, dogs, cats, horses, cows, goats, sheep, pigs, and rabbits. In one embodiment, the subject or patient is a human.

The terms “affected with a disease or disorder,” “afflicted with a disease or disorder,” or “having a disease or disorder” are used interchangeably herein and refer to a subject or patient with any disease, disorder, syndrome or condition. No increased or decreased level of severity of the disorder is implied by the use of one the terms as compared to the other.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

When introducing elements of the present disclosure or the preferred embodiment(s) thereof, the articles “a,” “an,” “the” and “said” are intended to mean that there are one or more of the elements. The terms “comprising,” “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e., A alone, B alone or A and B in combination. The expression “A, B and/or C” is intended to mean A alone, B alone, C alone, A and B in combination, A and C in combination, B and C in combination or A, B, and C in combination.

When ranges of values are disclosed, and the notation “from n1 . . . to n2” or “between n1 . . . and n2” is used, where n1 and n2 are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein in relation to a numerical value x means x±10%.

The term “comprising” encompasses “including” as well as “consisting” e.g., a composition “comprising” X may consist exclusively of X or may include something additional e.g., X+Y.

The word “substantially” does not exclude “completely” e.g., a composition which is “substantially free” from Y may be completely free from Y. Where necessary, the word “substantially” may optionally be omitted where used herein.

An “intention-to-treat” population includes all clinical trial subjects who are randomized according to randomized treatment assignment. Randomized controlled trials often suffer from two major complications, i.e., noncompliance and missing outcomes. One potential solution to this problem is a statistical concept called intention-to-treat (ITT) analysis. ITT analysis ignores noncompliance, protocol deviations, withdrawal, and anything that happens after randomization. ITT analysis maintains prognostic balance generated from the original random treatment allocation. In ITT analysis, estimate of treatment effect is generally conservative. A better application of the ITT approach is possible if complete outcome data are available for all randomized subjects. Per-protocol population is defined as a subset of the ITT population who completed the study without any major protocol violations. See, e.g., Gupta S K, Intention-to-treat concept: A review, Perspect Clin Res. 2011 July-September; 2(3): 109-112.

As used herein, “Thyroid Eye Disease” (TED), “Thyroid-associated Ophthalmopathy” (TAO), “Thyroid Inflammatory Eye Disease (TIED),” “Graves' Ophthalmopathy” (GO) or “Graves' Orbitopathy” (GO) refer to the same disorder or condition and are used interchangeably. They all refer to the inflammatory orbital pathology associated with some autoimmune thyroid disorders, most commonly with “Graves' Disease” (GD), but sometimes with other diseases, e.g. Hashimoto's thyroiditis.

The terms “proptosis” and “exophthalmos” (also known as exophthalmus, exophthalmia, or exorbitism) refer to the forward projection, displacement, bulging, or protrusion of an organ. As used herein, the terms refer to the forward projection, displacement, bulging, or protrusion of the eye anteriorly out of the orbit. Proptosis and exophthalmos are considered by some of skill in the art to have the same meaning and are often used interchangeably, while others attribute subtle differences to their meanings. Exophthalmos is used by some to refer to severe proptosis; or to refer to endocrine-related proptosis. Yet others use the term exophthalmos when describing proptosis associated with the eye, in, for example, subjects with TED (TAO or GO).

As used herein, the terms “proptosis” and “exophthalmos” are used interchangeably and refer to the forward projection, displacement, bulging, or protrusion of the eye anteriorly out of the orbit. Owing to the rigid bony structure of the orbit with only anterior opening for expansion, any increase in orbital soft tissue contents taking place from the side or from behind will displace the eyeball forward. Proptosis or exophthalmos can be the result of a several disease processes including infections, inflammations, tumors, trauma, metastases, endocrine lesions, vascular diseases & extra orbital lesions. TED (TAO or GO) is currently recognized as the most common cause of proptosis in adults. Exophthalmos can be either bilateral, as is often seen in TED (TAO or GO), or unilateral (as is often seen in an orbital tumor).

Measurement of the degree of exophthalmos can be performed using an exophthalmometer, an instrument used for measuring the degree of forward displacement of the eye. The device allows measurement of the forward distance of the lateral orbital rim to the front of the cornea.

Computed tomography (CT) scanning and Magnetic resonance imaging (MRI) may also be used in evaluating the degree of exophthalmos or proptosis. CT scanning is an excellent imaging modality for the diagnosis of TED (TAO or GO). In addition to allowing visualization of the enlarged extraocular muscles, CT scans provide the surgeon or clinician with depictions of the bony anatomy of the orbit when an orbital decompression is required. MRI, with its multi-planar and inherent contrast capabilities, provides excellent imaging of the orbital contents without the radiation exposure associated with CT scan studies. MRI provides better imaging of the optic nerve, orbital fat, and extraocular muscle, but CT scans provide better views of the bony architecture of the orbit.

Orbital ultrasonography can also be a used for the diagnosis and evaluation of TED (TAO or GO), because it can be performed quickly and with a high degree of confidence. High reflectivity and enlargement of the extraocular muscles are assessed easily, and serial ultrasonographic examinations can also be used to assess progression or stability of the ophthalmopathy.

Based on the technologies currently available, or that will become available in the future, one of skill in the art would be capable of determining the best modality for diagnosing and evaluating the extent of proptosis or exophthalmos.

Although it is generally accepted that the normal range of proptosis is 12-21 mm, it must be noted that the value for a normal person varies by age, gender and race. For example, in normal adult white males, the average distance of globe protrusion is 16.5 mm, with the upper limit of normal at 21.7 mm. In adult African Americans it averages 18.2 mm, with an upper normal limit of 24.1 mm in males and 22.7 mm in females. In Mexican adults, males averaged 15.2 mm and females averaged 14.8 mm and in Iran, for the age group of 20-70 years, the average was 14.7 mm. In Taiwanese adults, comparing normal subjects to those with Graves' Ophthalmopathy, the normal group had an average reading of 13.9 mm versus 18.3 mm for the TED group.

Even within a group of people, there can be variability. Four ethnic groups in Southern Thailand had exophthalmometry measurement averages ranging from 15.4 mm to 16.6 mm. In 2477 Turkish patients, the median measurement was 13 mm, with an upper limit of 17 mm; and in a Dutch study, the upper limit was 20 mm in males and 16 mm in females.

Although the average and upper limits for exophthalmos or proptosis vary widely, it is accepted in the field that a difference greater than 2 mm between the eyes is significant and not normal.

One of skill in the art, for example an ophthalmologist, surgeon or other clinician skilled in the knowledge and treatment of eye disorders would know what a normal value of proptosis is based on the age, gender and race of the subject and have the ability to diagnose or evaluate the presence or absence of proptosis as well as track its progression.

### Activity Measures or Assessments

Several classification systems have been conceived to assess the clinical manifestations of TED (TAO or GO). In 1969, Werner reported the NOSPECS Classification (No physical signs or symptoms, Only signs, Soft tissue involvement, Proptosis, Extraocular muscle signs, Corneal involvement, and Sight loss) (Werner, S. C. American Journal of Ophthalmology, 1969, 68, no. 4, 646-648.)

The modified NOSPECS was also published by Werner in 1977 and has been broadly used since then (Werner, S. C. American Journal of Ophthalmology, 1977, 83, no. 5, 725-727). This classification grades for clinical severity and does not provide a means of distinguishing active TED (inflammatory progressive) from inactive TED (noninflammatory stationary). Therefore, the indication for treatments used to be based exclusively in the severity of symptoms without consideration whether the disease was active or inactive. In 1989, Mourits et al. described the Clinical Activity Score (CAS) (Mourits et al., British Journal of Ophthalmology, 1989, 73, no. 8, 639-644) as a way of assessing the degree of active disease. This score, based on the classical signs of acute inflammation (pain, redness, swelling, and impaired function) was proposed as a clinical classification to discriminate easily between active and inactive disease and was modified in 1997 (Mourits et al., Clinical Endocrinology, 1997. 47, no. 1, 9-14). This protocol is further described below.

As used herein, the term CAS refers to the protocol described and scored as disclosed below. According to this protocol, one point is given for the presence of each of the parameters assessed in the list below. The sum of all points defines clinical activity and provides the CAS. For patients assessed for the first time only items 1-7 are scored. A CAS≥3/7 indicates active GO. For patients that are assessed for the second or subsequent time (typically, 1-3 months later), items 8-10 are also scored; and a CAS≥4/10 indicates active disease. A ten-item CAS scale exists as well, but in clinical trials, the 7-item scale is generally used, being more amenable to longitudinal studies involving multiple assessments.

The CAS consists of seven components:
1. spontaneous retrobulbar pain,
2. pain on attempted eye movements (upward, side-to-side, and downward gazes),
3. conjunctival redness,
4. redness of the eyelids,
5. chemosis (conjunctival swelling/edema),
6. swelling of the caruncle/plica, and
7. swelling of the eyelids.

Each component is scored as present (1 point) or absent (0 points). The score at each efficacy assessment is the sum of all items present; giving a range of 0-7, where 0 or 1 constitutes inactive disease and 7 severe active ophthalmopathy. A change of >2 points is considered clinically meaningful.

Item 1, spontaneous orbital pain could be a painful, or oppressive feeling on, or behind, the globe. This pain may be caused by the rise in intraorbital pressure, when the orbital tissues volume increases through excess synthesis of extracellular matrix, fluid accumulation, and cellular infiltration and expansion. Item 2, gaze evoked orbital pain, could be pain in the eyes when looking, or attempting to look, up, down or sideways, i.e., pain with upward, downward, or lateral eye movement, or when attempting upward, downward, or lateral gaze. This kind of pain could arise from the stretching of the inflamed muscle(s), especially on attempted up-gaze. The ‘stretching pain’ cannot be provoked by digital pressing on the eyeball, as would be expected if it were a manifestation of the raised intraorbital pressure. Both kinds of pain can be reduced after anti-inflammatory treatment. These kinds of pain are therefore considered to be directly related to autoimmune inflammation in the orbit and thus useful in assessing TED activity.

Swelling in TED (TAO or GO) is seen as chemosis (edema of the conjunctiva) and swelling of the caruncule and/or plica semilunaris. Both are signs of TED activity. Swollen eyelids can be caused by edema, fat prolapse through the orbital septum, or fibrotic degeneration. In addition to swelling, other symptoms indicative of active TED include redness and/or pain of the conjunctiva, eyelid, caruncule and/or plica semilunaris.

Other grading systems have also been developed for the assessment of TED (TAO or GO). The VISA Classification (vision, inflammation, strabismus, and appearance) (Dolman, P. J., and Rootman, J., Ophthalmic Plastic and Reconstructive Surgery, 2006, 22, no. 5, 319-324 and Dolman, P. J., Best Practice & Research Clinical Endocrinology & Metabolism, 2012, 26, no. 3, 229-248) and the European Group of Graves' Orbitopathy (EUGOGO) Classification (Bartalena, L., et al., European Journal of Endocrinology, 2008, 158, no. 3, 273-285) are two such examples. Both systems are grounded in the NO SPECS and CAS classifications and use indicators to assess the signs of activity and the degree of severity. More importantly, they allow the clinician to guide the treatment of the patient with GO. VISA is more commonly used in North America and Canada while EUGOGO is in Europe. Since the VISA and EUGOGO protocols are not interchangeable, only one of them should be employed as a reference in a specific patient.

### Graves Ophthalmopathy Quality of Life (GO-QoL)

In addition to proptosis (or exophthalmos) and CAS, quality of life (QoL) was also evaluated with the use of the Graves' ophthalmopathy quality of life (GO-QoL) questionnaire. This questionnaire is designed to determine the improved quality of life after treatment. In some embodiments, questionnaire may determine the decreased or lack of side effects after being treated with an antibody, or an antigen binding fragment thereof, according to the methods disclosed herein, as compared to treatment with glucocorticoids.

The GO-QoL questionnaire has two self-assessment subscales. The first relates to the impact of visual function on daily activities, while the second relates to the impact of self-perceived appearance. Each subscale has 8 questions which are answered with: (i) yes—very much so; (ii) yes—a little; or (iii) no—not at all. Each question is scored 0-2, respectively, and the total raw score is then mathematically transformed to a 0-100 scale, where 0 represents the most negative impact on quality of life, and 100 represents no impact. A change of >8 points on the 0-100 scale is considered to be clinically meaningful. The combined score takes raw scores from both subscales and again transforms them to a single 0-100 scale.

### Severity Measures

For lid aperture, the distance between the lid margins are measured (in mm) with the patient looking in the primary position, sitting relaxed, and with distant fixation.
For swelling of the eyelids, the measure/evaluation is either “absent/equivocal,” “moderate,” or “severe.”
Redness of the eyelids is either absent or present.
Redness of the conjunctivae is either absent or present.

Conjunctival edema is either absent or present.
Inflammation of the caruncle or plica is either absent or present.
Exophthalmos was measured in millimeter using the same Hertel exophthalmometer and same intercanthal distance for an individual patient.
Subjective diplopia is scored from 0 to 3 (0=no diplopia; 1=intermittent, i.e., diplopia in primary position of gaze, when tired or when first awakening; 2=inconstant, i.e., diplopia at extremes of gaze; 3=constant, i.e., continuous diplopia in primary or reading position).
For eye muscle involvement, the ductions are measured in degrees.
Corneal involvement is either absent/punctate or keratopathy/ulcer.
For optic nerve involvement, .i.e., best-corrected visual acuity, color vision, optic disc, relative afferent pupillary defect, the condition is either absent or present. In addition, visual fields are checked if optic nerve compression was suspected.

### Severity Classification

Sight-threatening thyroid eye disease: Patients with dysthyroid optic neuropathy (DON) and/or corneal breakdown. This category warranted immediate intervention.

Moderate-to-severe thyroid eye disease: Patients without sight-threatening disease whose eye disease had sufficient impact on daily life to justify the risks of immunosuppression (if active) or surgical intervention (if inactive). Patients with moderate-to-severe thyroid eye disease usually had any one or more of the following: lid retraction ≥2 mm, moderate or severe soft tissue involvement, exophthalmos ≥3 mm above normal for race and gender, inconstant or constant diplopia.

Mild thyroid eye disease: Patients whose features of thyroid eye disease have only a minor impact on daily life insufficient to justify immunosuppressive or surgical treatment. They usually have only one or more of the following: minor lid retraction (<2 mm), mild soft tissue involvement, exophthalmos <3 mm above normal for race and gender, transient or no diplopia, and corneal exposure responsive to lubricants.

### Assessment of Gorman Grading of Diplopia

The Gorman assessment of subjective diplopia includes four categories: no diplopia (absent), diplopia when the patient is tired or awakening (intermittent), diplopia at extremes of gaze (inconstant), and continuous diplopia in the primary or reading position (constant). Patients are scored according to which grade of diplopia they are experiencing. An improvement of ≥1 grade is considered clinically meaningful.

Additional testing, including clinical trial protocols and criteria and the lead-in study, which can be performed to determine efficacy for the treatment of TED can be found in US20190225696A1, which is hereby incorporated by reference in its entirety.

Further, the IGR-1R inhibitors described herein may be useful for the treatment of TED in subjects who were either proptosis non-responders (<2 mm reduction in proptosis in the study eye) in the lead-in study or were proptosis responders in the lead-in study but meet the criteria for re-treatment due to relapse.

### Antibodies

The sequences of the heavy chains and light chains of examples of antibodies that may be used in the methods disclosed herein, each comprising three CDRs on the heavy chain and three CDRs on the light chain are provided below. The sequences of the CDRs, heavy chains, light chains as well as the sequences of the nucleic acid molecules encoding the CDRs, heavy chains and light chains of the antibodies are disclosed in the sequence listing. The CDRs of the antibody heavy chains are referred to as CDRH1 (or HCDR1), CDRH2 (or HCDR2) and CDRH3 (or HCDR3), respectively. Similarly, the CDRs of the antibody light chains are referred to as CDRL1 (or LCDR1), CDRL2 (or LCDR2) and CDRL3 (or LCDR3), respectively.

Variant antibodies are also included within the scope of the disclosure. Thus, variants of the sequences recited in the application are also included within the scope of the disclosure. Such variants include natural variants generated by somatic mutation in vivo during the immune response or in vitro upon culture of immortalized B cell clones. Alternatively, variants may arise due to the degeneracy of the genetic code or may be produced due to errors in transcription or translation.

Further variants of the antibody sequences having improved affinity and/or potency may be obtained using methods known in the art and are included within the scope of the disclosure. For example, amino acid substitutions may be used to obtain antibodies with further improved affinity. Alternatively, codon optimization of the nucleotide sequence may be used to improve the efficiency of translation in expression systems for the production of the antibody. Further, polynucleotides comprising a sequence optimized for antibody specificity or neutralizing activity by the application of a directed evolution method to any of the nucleic acid sequences disclosed herein are also within the scope of the disclosure.

In one embodiment variant antibody sequences may share 70% or more (i.e. 75%, 80%, 85%, 90%, 95%, 97%, 98%, 99% or more) amino acid sequence identity with the sequences recited in the application. In some embodiments such sequence identity is calculated with regard to the full length of the reference sequence (i.e. the sequence recited in the application). In some further embodiments, percentage identity, as referred to herein, is as determined using BLAST version 2.1.3 using the default parameters specified by the NCBI (the National Center for Biotechnology Information; http://www.ncbi.nlm.nih.gov/) [Blosum 62 matrix; gap open penalty=11 and gap extension penalty=1].

Antibodies, or antigen binding fragments thereof, used with the methods disclosed herein can be of any isotype (e.g., IgA, IgG, IgM; i.e., an α, γ or μ heavy chain). In one embodiment the antibody is IgG. Within the IgG isotype, antibodies may be IgG1, IgG2, IgG3 or IgG4 subclass. The antibodies may have a κ or a λ light chain.

The antibodies, or an antigen binding fragments thereof, used with the methods disclosed herein can be administered by any route known to one of skill in the art.

### Antibody Fc Variants and Half-Life

In immunoglobulins, such as IgG, a site in the Fc region of the heavy chain mediates interaction with the neonatal receptor (FcRn). Binding to FcRn recycles endocytosed antibody from the endosome back to the bloodstream and plays a key role in antibody transport. This process, coupled with preclusion of kidney filtration due to the large size of the full-length molecule, results in favorable antibody serum half-lives ranging from one to three weeks in vivo. Thus, the fidelity of this region on Fc is important for the clinical properties of antibodies.

Other properties of the antibody may determine its clearance rate (e.g. stability and half-life) in vivo. In addition to antibody binding to the FcRn receptor, other factors that contribute to clearance and half-life are serum aggregation, enzymatic degradation in the serum, inherent immunogenicity of the antibody leading to clearing by the immune system, antigen-mediated uptake, FcR (non-FcRn) mediated uptake and non-serum distribution (e.g. in different tissue compartments).

Accordingly, one means by which the pharmacokinetics (PK) and pharmacodynamics (PD) of a therapeutic antibody can by changed is by increasing the serum half-life of the antibody by altering the heavy constant domains within the Fc. In addition, due to the methodologies outlined herein, the possibility of immunogenicity resulting from the FcRn variants is significantly reduced by importing variants from different IgG isotypes such that serum half-life is increased without introducing significant immunogenicity.

The substitutions in the Fc domains are chosen such that the resultant proteins show improved serum half-life in vivo as compared to the wild type protein. In order to increase the retention of the Fc proteins in vivo, the increase in binding affinity must be at around pH 6 while maintaining lower affinity at around pH 7.4. Without being limited to theory, Fc regions are believed to have longer half-lives in vivo because binding to FcRn at pH 6 in an endosome sequesters the Fc. The endosomal compartment then recycles the Fc to the cell surface. Once the compartment opens to the extracellular space, the higher pH (˜7.4) induces the release of Fc back into the blood. The increased affinity of Fc for FcRn at pH 7.4 is thought to forbid the release of the Fc back into the blood. As a result, Fc mutations that increase Fc's half-life in vivo generally increase FcRn binding at the lower pH while still allowing release of Fc at higher pH. The amino acid histidine changes its charge state in the pH range of 6.0 to 7.4. Therefore, it is not surprising to find histidine residues at important positions in the Fc/FcRn complex.

In some embodiments, the increase in FcRn binding over wild type specifically at lower pH (˜6.0) facilitates Fc/FcRn binding in the endosome. In some embodiments, Fc variants with altered FcRn binding can have altered binding to another class of Fc receptors, the FcγR's (FcgammaR's) as differential binding to FcγR5, particularly increased binding to FcγRIIIb and decreased binding to FcγRIIb, has been shown to result in increased efficacy.

In some embodiments, importation of substitutions at particular positions from one IgG isotype into another can be achieved, thus reducing or eliminating the possibility of unwanted immunogenicity being introduced into the variants. That is, IgG1 is a common isotype for therapeutic antibodies for a variety of reasons, including high effector function. IgG2 residues at particular positions can be introduced into the IgG1 backbone to result in a protein that exhibits longer serum half-life.

In some embodiments, non-isotypic amino acid changes are made, to improve binding to FcRn and/or to increase in vivo serum half-life, and/or to allow accommodations in structure for stability, etc.

As will be appreciated by those in the art and described below, a number of factors contribute to the in vivo clearance, and thus the half-life, of antibodies in serum. One factor involves the antigen to which the antibody binds; that is, antibodies with identical constant regions but different variable regions (e.g., Fv domains), may have different half-lives due to differential ligand binding effects. However, the present disclosure demonstrates that while the absolute half-life of two different antibodies may differ due to these antigen specificity effects, the FcRn variants described herein, can transfer to different ligands to give the same trends of increasing half-life. That is, in general, the relative “order” of the FcRn binding/half-life increases will track to antibodies with the same variants of antibodies with different Fvs as is discussed herein.

Fc variants within a therapeutic antibody are made by introducing amino acid mutations into the parent molecule. “Mutations” in this context are usually amino acid substitutions, although as shown herein, deletions and insertions of amino acids can also be done and thus are defined as mutations.

The Fc variant antibodies of the disclosure show increased binding to FcRn and/or increased in vivo serum half-life. By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. In some cases, the FcRn variants bind to the human FcRn receptor, or it may be desirable to design variants that bind to rodent or primate receptors in addition, to facilitate clinical trials.

In some embodiments, the present disclosure provides compositions and methods of administering an antibody to a subject, where the antibody comprises a variant Fc region as compared to a parent Fc region, wherein the variant Fc region comprises a first mutation that is a leucine at position 428 and a second mutation that is a serine at position 434, where the antibody has increased serum half-life as compared to an antibody comprising the parent Fc region, and wherein numbering is according to the EU index. In some embodiments, the antibody disclosed herein comprises a variant Fc region comprising mutations that substitute a methionine at position 428 with a leucine (Met428Leu) and substitutes an asparagine at position 434 with a serine (Asn434Ser). Numbering is EU as in Kabat, and it is understood that the substitution is non-native to the starting molecule. As has been shown previously, these FcRn substitutions work in IgG1, IgG2 and IgG1/G2 hybrid backbones, and are specifically included for IgG3 and IgG4 backbones and derivatives of any IgG isoform as well.

In some embodiments, the present disclosure provides compositions and methods of administering an antibody to a subject, where the antibody comprises a variant Fc region as compared to a parent Fc region, wherein the variant Fc region comprises a first mutation that is a tyrosine at position 252, a second mutation that is a threonine at position 254, and a third mutation that is a glutamic acid at position 256, where the antibody has increased serum half-life as compared to an antibody comprising the parent Fc region, and wherein numbering is according to the EU index. In some embodiments, the antibody disclosed herein comprises a variant Fc region comprising mutations that substitute a methionine at position 252 with a tyrosine (Met252Tyr), substitute a serine at position 254 with a threonine (Ser254Thr), and substitute a threonine at position 256 with a glutamic acid (Thr256Glu). Numbering is EU as in Kabat, and it is understood that the substitution is non-native to the starting molecule. As has been shown previously, these FcRn substitutions work in IgG1, IgG2 and IgG1/G2 hybrid backbones, and are specifically included for IgG3 and IgG4 backbones and derivatives of any IgG isoform as well.

The present disclosure includes variants of Fc domains, including those found in antibodies, Fc fusions, and immuno-adhesions, which have an increased binding to the FcRn receptor. As noted herein, binding to FcRn results in longer serum retention in vivo. A variety of such substitutions—including those related to M428L/N434S—are known and described in U.S. Pat. Nos. 7,317,091; 8,084,582; and 8,101,720; 8,188,231; 8,367,805; and 8,546,543, each of which is incorporated herein by reference in their entirety. Similarly, additional substitutions—including those related to M252Y/S254T/T256E—are known and described in International Patent Application No. WO/2002/060919 and U.S. Pat. No. 7,083,784, each of which is incorporated herein by reference in their entirety.

### Dosing and Administration

The compound, antibody, or an antigen binding fragment thereof, can be administered in a single dose or in multiple doses. In some embodiments, the therapeutic antibody is administered to the subject in a single dose. In some embodiments, the therapeutic antibody is administered to the subject in multiple doses, spread out over the course of a few days, weeks or months. In some embodiments the antibody, or an antigen binding fragment thereof, is administered every week or every 2 weeks or every 3 weeks or every 4 weeks or every 5 weeks or every 6 weeks or every 7 weeks or every 8 weeks or every month or every 2 months or every 3 months.

In some embodiments the antibody, or an antigen binding fragment thereof, is administered in multiple doses and the dosage is the same each time. In some embodiments the antibody, or an antigen binding fragment thereof, is administered in multiple doses and the dosage at the time of first administration is different (could be higher or lower) from those at subsequent times. In some embodiments the antibody, or an antigen binding fragment thereof, is administered in multiple doses and the dosage is adjusted at each administration based on the subject's response to the therapy.

The dosage may further vary between patients, based on different factors such as the age, gender, race, and body weight of each patient. In some embodiments, the dosage varies by body weight of the patient. The dosage could range from about 1 mg of the antibody, or an antigen binding fragment thereof, per kilogram of body weight to about 100 mg of the antibody, or an antigen binding fragment thereof, per kilogram of body weight. The dosage, could for example, be 1 mg, 2 mg, 3 mg, 5 mg, 7 mg, 10 mg, 12 mg, 15 mg, 17 mg, 20 mg, 22 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg or 100 mg, of the antibody, or an antigen binding fragment thereof, per kilogram of body weight.

In some embodiments, the dose is about 0.3 mg/kg to about 10 mg/kg of the antibody, or an antigen binding fragment thereof. In some embodiments, the dosage is about 0.3 mg/kg to about 5 mg/kg of the antibody, or an antigen binding fragment thereof. In some embodiments, the dosage is about 0.3 mg/kg to about 1 mg/kg of the antibody, or an antigen binding fragment thereof. The dosage, could for example, be about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, 1.6 mg/kg, 1.7 mg/kg, 1.8 mg/kg, 1.9 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, or about 10 mg/kg, or any number of tenths of a mg/kg in between the foregoing, of the antibody, or an antigen binding fragment thereof. In some embodiments, the dose is administered every week.

In some embodiments, the dose is about 0.6 mg/kg to about 20 mg/kg of the antibody, or an antigen binding fragment thereof. In some embodiments, the dosage is about 0.6 mg/kg to about 5 mg/kg of the antibody, or an antigen binding fragment thereof. In some embodiments, the dosage is about 0.6 mg/kg to about 10 mg/kg of the antibody, or an antigen binding fragment thereof. The dosage, could for example, be about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, 1.6 mg/kg, 1.7 mg/kg, 1.8 mg/kg, 1.9 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg, or any number of tenths of a mg/kg in between the foregoing, of the antibody, or an antigen binding fragment thereof. In some embodiments, the dose is administered every two weeks.

In some embodiments, the dose is about 1 mg/kg to about 30 mg/kg of the antibody, or an antigen binding fragment thereof. In some embodiments, the dose is about 5 mg/kg to about 30 mg/kg of the antibody, or an antigen binding fragment thereof. In some embodiments, the dose is about 10 mg/kg to about 30 mg/kg of the antibody, or an antigen binding fragment thereof. The dosage, could for example, be about 1 mg/kg, about 2 mg/kg, about 3 mg/kg, about 5 mg/kg, about 7 mg/kg, about 10 mg/kg, about 12 mg/kg, about 15 mg/kg, about 17 mg/kg, about 20 mg/kg, about 22 mg/kg, about 25 mg/kg, or about 30 mg/kg, or any integer and/or number of tenths of a mg/kg in between the foregoing, of the antibody, or an antigen binding fragment thereof. In some embodiments, the dose is administered every three weeks.

In some embodiments, the dose is about 1.2 mg/kg to about 40 mg/kg of the antibody, or an antigen binding fragment thereof. In some embodiments, the dose is about 5 mg/kg to about 40 mg/kg of the antibody, or an antigen binding fragment thereof. In some embodiments, the dose is about 10 mg/kg to about 40 mg/kg of the antibody, or an antigen binding fragment thereof. In some embodiments, the dose is about 20 mg/kg to about 40 mg/kg of the antibody, or an antigen binding fragment thereof. In some embodiments, the dose is about 25 mg/kg to about 40 mg/kg of the antibody, or an antigen binding fragment thereof. The dosage, could for example, be about 1 mg/kg, about 2 mg/kg, about 3 mg/kg, about 5 mg/kg, about 7 mg/kg, about 10 mg/kg, about 12 mg/kg, about 15 mg/kg, about 17 mg/kg, about 20 mg/kg, about 22 mg/kg, about 25 mg/kg, about 27 mg/kg, about 30 mg/kg, about 32 mg/kg, about 35 mg/kg, about 37 mg/kg, or about 40 mg/kg, or any integer and/or number of tenths of a mg/kg in between the foregoing, of the antibody, or an antigen binding fragment thereof. In some embodiments, the dose is administered every four weeks.

In some embodiments, the dosage is about 1 mg/kg to about 5 mg/kg of the antibody, or an antigen binding fragment thereof. In some embodiments, the dosage is about 5 mg/kg to about 10 mg/kg of the antibody, or an antigen binding fragment thereof. In some embodiments, the dosage is about 10 mg/kg to about 15 mg/kg of the antibody, or an antigen binding fragment thereof. In some embodiments, the dosage is about 15 mg/kg to about 20 mg/kg of the antibody, or an antigen binding fragment thereof.

In some embodiments, the dosage is about 1 mg/kg to about 5 mg/kg of the antibody, or an antigen binding fragment thereof. In some embodiments, the dosage is about 5 mg/kg to about 10 mg/kg of the antibody, or an antigen binding fragment thereof. In some embodiments, the dosage is about 10 mg/kg to about 15 mg/kg of the antibody, or an antigen binding fragment thereof. In some embodiments, the dosage is about 15 mg/kg to about 20 mg/kg of the antibody, or an antigen binding fragment thereof.

In some embodiments the antibody, or an antigen binding fragment thereof, is administered in multiple doses and the dosage at the time of first administration is different from those at subsequent times, the dosage at the time of first administration is about 1 mg/kg to about 5 mg/kg of the antibody, or an antigen binding fragment thereof; or about 5 mg/kg to about 10 mg/kg of the antibody, or an antigen binding fragment thereof; or about 10 mg/kg to about 15 mg/kg of the antibody, or an antigen binding fragment thereof; or about 15 mg/kg to about 20 mg/kg of the antibody, or an antigen binding fragment thereof; or about 20 mg/kg to about 25 mg/kg of the antibody, or an antigen binding fragment thereof. The subsequent dose(s) could be higher or lower than the first dose. In some embodiments, the subsequent dose is about 1 mg/kg to about 5 mg/kg of the antibody, or an antigen binding fragment thereof; or about 5 mg/kg to about 10 mg/kg of the antibody, or an antigen binding fragment thereof; or about 10 mg/kg to about 15 mg/kg of the antibody, or an antigen binding fragment thereof; or about 15 mg/kg to about 20 mg/kg of the antibody, or an antigen binding fragment thereof; or about 20 mg/kg to about 25 mg/kg of the antibody, or an antigen binding fragment thereof.

Small molecule compounds may be administered orally, via injection, etc. at a dose of from 0.01 to 500 mg/kg per day and/or from 0.1 mg to 5 g per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, for example around 10 mg to 200 mg.

The amount of active ingredient that may be combined with the carrier materials to produce a single dosage form will vary depending upon the host treated and the particular mode of administration. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

Additional dosage ranges are provided throughout this disclosure.

The duration of the treatment depends on the subject's response to the therapy and can range from about one month or 4 weeks to about 2 years or 100 weeks. In some embodiments, the treatment may be provided over a total duration of about 1 month, 2 months, 3 months, 4 months, 5 months, 6 months, 7 months, 8 months, 9 months, 10 months, 11 months, 1 year, 14 months, 16 months, 18 months, 20 months, 22 months or 2 years. In some embodiments, the treatment may be provided over a total duration of 4, 6, 8, 10, 12, 14, 16, 20, 22, 24, 26, 28, 30, 32, 34, 36, 38, 40, 42, 44, 46, 48, 50, 52 weeks, or extended to 56, 64, 72, 80, 88, 96 or 104 weeks.

In some embodiments, the antibody, or an antigen binding fragment thereof, is administered for a duration of 24 weeks at intervals of 3 weeks starting with an initial dose of 10 mg per kilogram of body weight, followed by 20 mg per kilogram for seven additional treatments. In some embodiments, the molecule compound is administered daily (QD), twice daily, (BID) or thrice daily (TID) for an appropriate duration, e.g., 24 weeks.

The compound, antibody, or an antigen binding fragment thereof, may be administered by any suitable route including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes. Hyposprays may also be used to administer the pharmaceutical compositions disclosed herein. Typically, the therapeutic antibody may be prepared as a freeze-dried (lyophilized) powder or as an injectable, either as a liquid solution or suspension. Solid forms suitable for solution in, or suspension in, liquid vehicles prior to injection may also be used.

### Pharmaceutical Compositions

The pharmaceutical compositions used in the methods disclosed herein comprise one or more of: the antibodies or antibody fragments described above and a pharmaceutically acceptable carrier or excipient. Although the carrier or excipient may facilitate administration, it should not itself induce the production of antibodies harmful to the subject or individual receiving the composition; nor should it be toxic. Suitable carriers may be large, slowly metabolized macromolecules such as proteins, polypeptides, liposomes, polysaccharides, polylactic acids, polyglycolic acids, polymeric amino acids, amino acid copolymers and inactive virus particles, and are known to one of skill in the art.

The antibodies, or an antigen binding fragments thereof, or pharmaceutical compositions used with the methods disclosed herein may be administered by any number of routes including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes. Hyposprays may also be used to administer the pharmaceutical compositions disclosed herein. Typically, the therapeutic compositions may be prepared as injectables, either as liquid solutions or suspensions. Solid forms suitable for solution in, or suspension in, liquid vehicles prior to injection may also be prepared.

In one embodiment, the antibody, or an antigen binding fragment thereof, or pharmaceutical composition is administered intravenously. In another embodiment, the antibody, or an antigen binding fragment thereof, or pharmaceutical composition is administered by intravenous infusion.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule. Known antibody-based pharmaceuticals provide guidance relating to frequency of administration e.g., whether a pharmaceutical should be delivered daily, weekly, monthly, etc. Frequency and dosage may also depend on the severity of symptoms.

It will be appreciated that the active ingredient in the composition will be an antibody molecule, an antibody fragment or variants and derivatives thereof. As such, it will be susceptible to degradation in the gastrointestinal tract. Thus, if the composition is to be administered by a route using the gastrointestinal tract, the composition will need to contain agents which protect the antibody from degradation, but which release the antibody once it has been absorbed from the gastrointestinal tract.

For larger molecular weight moieties such as mAbs (˜150 kDa), the SC capillaries have low passive permeability; absorption of mAbs into systemic circulation occurs via lymphatic uptake from the interstitial space, as well as via active transport by the neonatal Fc receptor (FcRn) across the capillary endothelia. The extracellular matrix of the subcutaneous tissue also limits the injection of larger volumes (>1-2 mL) SC generally; coformulation with a recombinant hyaluronidase or soluble fragment thereof such as rHuPH20 can permit higher bioavailability. Additionally, physiochemical properties of mAbs, including charge, hydrophobicity, and stability, affect the rate and extent of their SC absorption; for example, the combination of high positive charge and hydrophobic interaction can reduce the rate absorption.

In some embodiments, pharmaceutical compositions suitable for use in the methods disclosed herein are formulated for subcutaneous administration. Examples of formulations suitable for subcutaneous administration include, but are not limited to, solutions, suspensions, emulsions, and dry products that can be dissolved or suspended in a pharmaceutically acceptable carrier for injection. Antibodies have been, and may be, formulated for subcutaneous administration using methods known in the art.

Pharmaceutical compositions suitable for use in the methods disclose herein comprise one or more pharmaceutically acceptable carriers, such as those widely employed in the art of drug manufacturing, and particularly antibody drug manufacturing. Pharmaceutically acceptable carriers in particular are non-toxic and should not interfere with the efficacy of the active ingredient. The carrier may be a diluent, adjuvant, excipient, or vehicle with which the antibodies are administered. Such vehicles may be liquids, such as aqueous fluids, oils, and emulsions. For example, 0.4% saline and 0.3% glycine may be used. The solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the antibodies in such pharmaceutical formulation may vary and will be selected primarily based on required dose, fluid volumes, viscosities, etc., according to the particular mode of administration selected, and other concerns, such as protein aggregation.

Examples of pharmaceutically acceptable carriers are solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, permeation enhancers, or combinations thereof.

Examples of buffers that may be used are acetic acid, citric acid, formic acid, succinic acid, phosphoric acid, carbonic acid, malic acid, aspartic acid, histidine, boric acid, Tris buffers, HEPPSO and HEPES.

Examples of antioxidants that may be used are ascorbic acid, methionine, cysteine hydrochloride, sodium bisulfate, sodium metabi sulfite, sodium sulfite, lecithin, citric acid, ethylenediamine tetraacetic acid (EDTA), sorbitol and tartaric acid.

Examples of amino acids that may be used are histidine, isoleucine, methionine, glycine, arginine, lysine, L-leucine, tri-leucine, alanine, glutamic acid, L-threonine, and 2-phenylamine.

Examples of surfactants that may be used are polysorbates (e.g., polysorbate-20 or polysorbate-80); polyoxamers (e.g, poloxamer 188); Triton; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g, lauroamidopropyl); myristamidopropyl-, palmidopropyl-, or isostearamidopropyl-dimethylamine; sodium methyl cocoyl-, or disodium methyl oleyl-taurate; and the MONAQUA™ series (Mona Industries, Inc., Paterson, N. J.), polyethyl glycol, polypropyl glycol, and copolymers of ethylene and propylene glycol (e.g., PLURONICS™, PF68, etc.).

Examples of preservatives that may be used are phenol, m-cresol, p-cresol, o-cresol, chlorocresol, benzyl alcohol, phenylmercuric nitrite, phenoxyethanol, formaldehyde, chlorobutanol, magnesium chloride, alkylparaben (methyl, ethyl, propyl, butyl and the like), benzalkonium chloride, benzethonium chloride, sodium dehydroacetate and thimerosal, or mixtures thereof.

Examples of saccharides that may be used are monosaccharides, disaccharides, trisaccharides, polysaccharides, sugar alcohols, reducing sugars, nonreducing sugars such as glucose, sucrose, trehalose, lactose, fructose, maltose, dextran, glycerin, dextran, erythritol, glycerol, arabitol, sylitol, sorbitol, mannitol, mellibiose, melezitose, raffmose, mannotriose, stachyose, maltose, lactulose, maltulose, glucitol, maltitol, lactitol or iso-maltulose.

Examples of permeation enhancers that may be used include recombinant hyaluronidase or soluble fragment thereof such as rHuPH20 (Halozyme). Liquid formulations for subcutaneous administration may comprise rHuPH20 or another soluble human hyaluronidase enzyme. rHuPH20 may be present in an amount sufficient to result in an increase in the dispersion of the antibodies contained in the same liquid formulation during subcutaneous administration.

The amounts of pharmaceutically acceptable carrier(s) in the pharmaceutical compositions may be determined experimentally based on the activities of the carrier(s) and the desired characteristics of the formulation, such as stability, bioavailability, and/or minimal oxidation.

The methods of the present disclosure can use an antibody, or an antigen binding fragment thereof, as described above, alone or in combination with other pharmaceutically active compounds, to treat conditions such as those disclosed hereinabove. The additional pharmaceutically active compound(s) can be administered simultaneously (either in the same dosage form or in separate dosage forms) or sequentially. Accordingly, in one embodiment, the present disclosure comprises methods for treating a condition by administering to the subject a therapeutically-effective amount of an antibody, or an antigen binding fragment thereof, of the present disclosure and one or more additional pharmaceutically active compounds.

In some embodiments, the antibody, or an antigen binding fragment thereof, of the present disclosure is used in combination with existing therapies, including, but not limited to, corticosteroids; rituximab and other anti-CD20 antibodies; tocilizumab and other anti-IL-6 antibodies; or selenium, infliximab and other anti-TNF-alpha antibodies. In some embodiments, the antibody, or an antigen binding fragment thereof, of the present disclosure is used in combination with TSHR inhibitors.

### EXAMPLES

Exemplary embodiments are provided in the following Examples 1-X. The following examples are presented only by way of illustration and to assist one of ordinary skill in using the invention. The examples are not intended in any way to otherwise limit the scope of the invention. In some embodiments, said IGF-1R inhibitor is an antibody or a subset of antibodies chosen from amongst the Examples below. In some embodiments, said IGF-1R inhibitor is a small molecule or a subset of small molecules chosen from amongst the Examples below.

### Example A

### Teprotumumab

Provided first is teprotumumab (TEPEZZA), an IGF-1R inhibitor approved for the treatment of TED. Teprotumumab and other related IGF-1R inhibitor antibodies and their methods of preparation can be found in U.S. Pat. No. 7,572,897, US20190225696, and US20190270820, which are hereby incorporated by reference in their entireties. In certain embodiments, teprotumumab may be used as an active control in clinical trials of other IGF-1R inhibitors, e.g. as in Example 31.

TABLE A
Teprotumumab Sequences and SEQ ID Numbers

| SEQ | | |
|---|---|---|
| ID | | |
| NO | Description | Sequence |
| | Antibody 1 | |
| | (teprotumumab) | |
| 84 | CDRH1 aa | Ser Tyr Gly Met His |
| 85 | CDRH2 aa | Ile Ile Trp Phe Asp Gly Ser Ser Thr Tyr Tyr Ala Asp Ser Val |
| | | Arg Gly |
| 86 | CDRH3 aa | Glu Leu Gly Arg Arg Tyr Phe Asp Leu |
| 87 | CDRL1 aa | Arg Ala Ser Gln Ser Val Ser Ser Tyr Leu Ala |
| 88 | CDRL2 aa | Asp Ala Ser Lys Arg Ala Thr |
| 89 | CDRL3 aa | Gln Gln Arg Ser Lys Trp Pro Pro Trp Thr |
| 90 | VH aa | Gln Val Glu Leu Val Glu Ser Gly Gly Gly Val Val Gln Pro Gly |
| | | Arg Ser Gln Arg Leu Ser Cys Ala Ala Ser Gly Phe Thr Phe Ser |
| | | Ser Tyr Gly Met His Trp Val Arg Gln Ala Pro Gly Lys Gly |
| | | Leu Glu Trp Val Ala Ile Ile Trp Phe Asp Gly Ser Ser Thr Tyr |
| | | Tyr Ala Asp Ser Val Arg Gly Arg Phe Thr Ile Ser Arg Asp Asn |
| | | Ser Lys Asn Thr Leu Tyr Leu Gln Met Asn Ser Leu Arg Ala |
| | | Glu Asp Thr Ala Val Tyr Phe Cys Ala Arg Glu Leu Gly Arg |
| | | Arg Tyr Phe Asp Leu Trp Gly Arg Gly Thr Leu Val Ser Val |
| | | Ser Ser |
| 91 | VL aa | Glu Ile Val Leu Thr Gln Ser Pro Ala Thr Leu Ser Leu Ser Pro |
| | | Gly Glu Arg Ala Thr Leu Ser Cys Arg Ala Ser Gln Ser Val |
| | | Ser Ser Tyr Leu Ala Trp Tyr Gln Gln Lys Pro Gly Gln Ala Pro |
| | | Arg Leu Leu Ile Tyr Asp Ala Ser Lys Arg Ala Thr Gly Ile Pro |
| | | Ala Arg Phe Ser Gly Ser Gly Ser Gly Thr Asp Phe Thr Leu Thr |
| | | Ile Ser Ser Leu Glu Pro Glu Asp Phe Ala Val Tyr Tyr Cys Gln |
| | | Gln Arg Ser Lys Trp Pro Pro Trp Thr Phe Gly Gln Gly Thr Lys |
| | | Val Glu Ser Lys |
| | Antibody 2 | |
| 84 | CDRH1 aa | Ser Tyr Gly Met His |
| 92 | CDRH2 aa | Ile Ile Trp Phe Asp Gly Ser Ser Lys Tyr Tyr Gly Asp Ser Val |
| | | Lys Gly |
| 86 | CDRH3 aa | Glu Leu Gly Arg Arg Tyr Phe Asp Leu |
| 87 | CDRL1 aa | Arg Ala Ser Gln Ser Val Ser Ser Tyr Leu Ala |
| 93 | CDRL2 aa | Asp Ala Ser Asn Arg Ala Thr |
| 89 | CDRL3 aa | Gln Gln Arg Ser Lys Trp Pro Pro Trp Thr |
| 94 | VH aa | Gln Val Gln Leu Val Glu Ser Gly Gly Gly Val Val Gln Pro Gly |
| | | Arg Ser Leu Arg Leu Ser Cys Ala Ala Ser Gly Phe Thr Phe Ser |
| | | Ser Tyr Gly Met His Trp Val Arg Gln Ala Pro Gly Lys Gly |
| | | Leu Glu Trp Met Ala Ile Ile Trp Phe Asp Gly Ser Ser Lys Tyr |
| | | Tyr Gly Asp Ser Val Lys Gly Arg Phe Thr Ile Ser Arg Asp Asn |
| | | Ser Lys Asn Thr Leu Tyr Leu Gln Met Asn Ser Leu Arg Ala |
| | | Asp Asp Thr Ala Val Tyr Tyr Cys Ala Arg Glu Leu Gly Arg |
| | | Arg Tyr Phe Asp Leu Trp Gly Arg Gly Thr Leu Val Thr Val |
| | | Ser Ser |
| 95 | VL aa | Glu Ile Val Leu Thr Gln Ser Pro Ala Thr Leu Ser Leu Ser Pro |
| | | Gly Glu Arg Ala Thr Leu Ser Cys Arg Ala Ser Gln Ser Val Ser |
| | | Ser Tyr Leu Ala Trp Tyr Gln Gln Lys Pro Gly Gln Ala Pro Arg |
| | | Leu Leu Ile Tyr Asp Ala Ser Asn Arg Ala Thr Gly Ile Pro Ala |
| | | Arg Phe Ser Gly Ser Gly Ser Gly Thr Asp Phe Thr Leu Thr Ile |
| | | Ser Ser Leu Glu Pro Glu Asp Phe Ala Val Tyr Tyr Cys Gln Gln |
| | | Arg Ser Lys Trp Pro Pro Trp Thr Phe Gly Gln Gly Thr Lys Val |
| | | Glu Ile Lys |

### Example 1

### Dalotuzumab

Dalotuzumab and other related IGF-1R inhibitor antibodies and their methods of preparation can be found in WO 2005/058967, which is hereby incorporated by reference in its entirety.

Heavy Chain CDRs-Dalotuzumab

| HCDR1 | HCDR2 | HCDR3 |
|---|---|---|
| GGYLWN | YISYDGTNNYKPSLKD | YGRVFFDY |
| (SEQ ID NO: 1) | (SEQ ID NO: 2) | (SEQ ID NO: 3) |

Light Chain CDRs-Dalotuzumab

| LCDR1 | LCDR2 | LCDR3 |
|---|---|---|
| RSSQSIVHSNGNTYLQ | KVSNRLY | FQGSHVPWT |
| (SEQ ID NO: 4) | (SEQ ID NO: 5) | (SEQ ID NO: 6) |

| | |
|---|---|
| Heavy Chain | QVQLQESGPGLVKPSETLSLTCTVS |
| (HC) | GYSITGGYLWNWIRQPPGKGLEWIG |
| | YISYDGTNNYKPSLKDRVTISRDTS |
| | KNQFSLKLSSVTAADTAVYYCARYG |
| | RVFFDYWGQGTLVTVSSASTKGPSV |
| | FPLAPSSKSTSGGTAALGCLVKDYF |
| | PEPVTVSWNSGALTSGVHTFPAVLQ |
| | SSGLYSLSSVVTVPSSSLGTQTYIC |
| | NVNHKPSNTKVDKRVEPKSCDKTHT |
| | CPPCPAPELLGGPSVFLFPPKPKDT |
| | LMISRTPEVTCVVVDVSHEDPEVKF |
| | NWYVDGVEVHNAKTKPREEQYNSTY |
| | RVVSVLTVLHQDWLNGKEYKCKVSN |
| | KALPAPIEKTISKAKGQPREPQVYT |
| | LPPSREEMTKNQVSLTCLVKGFYPS |
| | DIAVEWESNGQPENNYKTTPPVLDS |
| | DGSFFLYSKLTVDKSRWQQGNVFSC |
| | SVMHEALHNHYTQKSLSLSPGK |
| | (SEQ ID NO: 7) |
| Light Chain | DIVMTQSPLSLPVTPGEPASISCRS |
| (LC) | SQSIVHSNGNTYLQWYLQKPGQSPQ |
| | LLIYKVSNRLYGVPDRFSGSGSGTD |
| | FTLKISRVEAEDVGVYYCFQGSHVP |
| | WTFGQGTKVEIKRTVAAPSVFIFPP |
| | SDEQLKSGTASVVCLLNNFYPREAK |
| | VQWKVDNALQSGNSQESVTEQDSKD |
| | STYSLSSTLTLSKADYEKHKVYACE |
| | VTHQGLSSPVTKSFNRGEC |
| | (SEQ ID NO: 8) |

Some embodiments of the disclosure are anti-IGF-1R inhibitor mAbs or antigen binding fragments thereof, comprising a heavy chain comprising a variable heavy chain CDR1, a variable heavy chain CDR2, and a variable heavy chain CDR3, wherein the variable heavy chain CDR1 comprises an amino acid sequence SEQ ID NO:1, the variable heavy chain CDR2 comprises an amino acid sequence SEQ ID NO:2; and the variable heavy chain CDR3 comprises an amino acid sequence SEQ ID NO:3 or at least a CDR with at least 80% of sequence identity after optimal alignment with SEQ ID NO:1, SEQ ID NO:2, and SEQ ID NO:3.

The anti-IGF-1R inhibitor mAbs or antibody or antigen binding fragment thereof may additionally comprise a light chain which is paired with the heavy chain to form an antigen binding domain. In some embodiments, the light chain comprises a variable light chain CDR1, a variable light chain CDR2, and a variable light chain CDR3, wherein the variable light chain CDR1 comprises an amino acid sequence SEQ ID NO:4, the variable light chain CDR2 comprises an amino acid sequence SEQ ID NO:5; and the variable light chain CDR3 comprises an amino acid sequence SEQ ID NO:6 or at least a CDR with at least 80% of homology after optimal alignment with SEQ ID NO:4, SEQ ID NO:5, and SEQ ID NO:6.

In some embodiments, the anti-IGF-1R inhibitor mAbs or antigen binding fragment thereof comprises a heavy chain amino acid sequence of SEQ ID NO:7 or at least a heavy chain with at least 85%, 90%, 95%, 97%, 98%, or 99% of sequence identity after optimal alignment with SEQ ID NO:7. Alternatively, or in addition, the anti-IGF-1R inhibitor mAbs or antigen binding fragment thereof may comprise a light chain having an amino acid sequence of SEQ ID NO:8 or at least a heavy chain with at least 85%, 90%, 95%, 97%, 98%, or 99% of sequence identity after optimal alignment with SEQ ID NO:8.

### Example 2

### Ganitumab

Ganitumab and other related IGF-1R inhibitor antibodies and their methods of preparation can be found in WO 2006/069202, which is hereby incorporated by reference in its entirety.

Heavy Chain CDRs-Ganitumab

| HCDR1 | HCDR2 | HCDR3 |
|---|---|---|
| SSNWWS | EIYHSGSTNYNPSLKS | WTGRTDAFDI |
| (SEQ ID NO: 9) | (SEQ ID NO: 10) | (SEQ ID NO: 11) |

Light Chain CDRs-Ganitumab

| LCDR1 | LCDR2 | LCDR3 |
|---|---|---|
| ISCRSSQSLLHSNGYNYLD | LGSNRAS | MQGTHWPLT |
| (SEQ ID NO: 12) | (SEQ ID NO: 13) | (SEQ ID NO: 14) |
| Heavy Chain | QVQLQES | |
| (HC) | GPGLVKPSGTLSLTCAVSGGSISSSNWWSWVRQPPGKGLEW | |
| | IGEIYHSGSTNYNPSLKSRVTSVDKSKNQFSLKLSSVTAAD | |
| | TAVYYCARWTGRTDAFDIWGQGTMVTVSSASTKGPSVFPL | |
| | APSSKSTSGGTAALGCLVKDYFPEPVTVSWNSGALTSGVH | |
| | TFPAVLQSSGLYSLSSVVTVPSSSLGTQTYICNVNHKPSN | |
| | TKVDKKVEPKSCDKTHTCPPCPAPELLGGPSVFLFPPKPK | |
| | DTLMISRTPEVTCVVVDVSHEDPEVKFNWYVDGVEVHNAK | |
| | TKPREEQYNSTYRVVSVLTVLHQDWLNGKEYKCKVSNKAL | |
| | PAPIEKTISKAKGQPREPQVYTLPPSRDELTKNQVSLTCL | |
| | VKGFYPSDIAVEWESNGQPENNYKTTPPVLDSDGSFFLYS | |
| | KLTVDKSRWQQGNVFSCSVMHEALHNHYTQKSLSLSPGK | |
| | (SEQ ID NO: 15) | |
| Light Chain | DVVMTQSPLSLPVTPGEPASISCRSSQSLLHSNGYNYLDW | |
| (LC) | YLQKPGQSPQLLIYLGSNRASGVPDRFSGSGSGTDFTLKI | |
| | SRVEAEDVGVYYCMQGTHWPLTFGQGTKVEIKRTVAAPSV | |
| | FIFPPSDEQLKSGTASVVCLLNNFYPREAKVQWKVDNALQ | |
| | SGNSQESVTEQDSKDSTYSLSSTLTLSKADYEKHKVYACE | |
| | VTHQGLSSPVTKSENRGEC (SEQ ID NO: 16) | |

Some embodiments of the disclosure are anti-IGF-1R inhibitor mAbs or antigen binding fragments thereof, comprising a heavy chain comprising a variable heavy chain CDR1, a variable heavy chain CDR2, and a variable heavy chain CDR3, wherein the variable heavy chain CDR1 comprises an amino acid sequence SEQ ID NO:9, the variable heavy chain CDR2 comprises an amino acid sequence SEQ ID NO: 10; and the variable heavy chain CDR3 comprises an amino acid sequence SEQ ID NO:11 or at least a CDR with at least 80% of sequence identity after optimal alignment with SEQ ID NO:9, SEQ ID NO:10, and SEQ ID NO:11.

The anti-IGF-1R inhibitor mAbs or antibody or antigen binding fragment thereof may additionally comprise a light chain which is paired with the heavy chain to form an antigen binding domain. In some embodiments, the light chain comprises a variable light chain CDR1, a variable light chain CDR2, and a variable light chain CDR3, wherein the variable light chain CDR1 comprises an amino acid sequence SEQ ID NO:12, the variable light chain CDR2 comprises an amino acid sequence SEQ ID NO:13; and the variable light chain CDR3 comprises an amino acid sequence SEQ ID NO:14 or at least a CDR with at least 80% of homology after optimal alignment with SEQ ID NO:12, SEQ ID NO:13, and SEQ ID NO:14.

In some embodiments, the anti-IGF-1R inhibitor mAbs or antigen binding fragment thereof comprises a heavy chain amino acid sequence of SEQ ID NO:15 or at least a heavy chain with at least 85%, 90%, 95%, 97%, 98%, or 99% of sequence identity after optimal alignment with SEQ ID NO:15. Alternatively, or in addition, the anti-IGF-1R inhibitor mAbs or antigen binding fragment thereof may comprise a light chain having an amino acid sequence of SEQ ID NO:16 or at least a heavy chain with at least 85%, 90%, 95%, 97%, 98%, or 99% of sequence identity after optimal alignment with SEQ ID NO:16.

### Example 3

### Xentuzumab

Xentuzumab and other related IGF-1R inhibitor antibodies and their methods of preparation can be found in WO 2014/135611, which is hereby incorporated by reference in its entirety.

Heavy Chain CDRs-Xentuzumab

| HCDR1 | HCDR2 | HCDR3 |
|---|---|---|
| SYWMS | SITSYGSFTYADSVK | NMYTHFDS |
| (SEQ ID NO: 17) | (SEQ ID NO: 18) | (SEQ ID NO: 19) |

Light Chain CDRs-Xentuzumab

| LCDR1 | LCDR2 | LCDR3 |
|---|---|---|
| SGSSSNIGSNSVS | DNSKRPS | QSRDTYGYYWV |
| (SEQ ID NO: 20) | (SEQ ID NO: 21) | (SEQ ID NO: 22) |

| | |
|---|---|
| Heavy Chain | QVELVESGGGLVQPGGSLRLSCAASGFTFTSYWMSWVRQA |
| (HC) | PGKGLELVSSITSYGSFTYYADSVKGRFTISRDNSKNTLY |
| | LQMNSLRAEDTAVYYCARNMYTHFDSWGQGTLVTVSSAST |
| | KGPSVFPLAPSSKSTSGGTAALGCLVKDYFPEPVTVSWNS |
| | GALTSGVHTFPAVLQSSGLYSLSSVVTVPSSSLGTQTYIC |
| | NVNHKPSNTKVDKKVEPKSCDKTHTCPPCPAPELLGGPSV |
| | FLFPPKPKDTLMISRTPEVTCVVVDVSHEDPEVKFNWYVD |
| | GVEVHNAKTKPREEQYNSTYRVVSVLTVLHQDWLNGKEYK |
| | CKVSNKALPAPIEKTISKAKGQPREPQVYTLPPSRDELTK |
| | NQVSLTCLVKGFYPSDIAVEWESNGQPENNYKTTPPVLDS |
| | (SEQ ID NO: 23) |
| Light Chain | DIVLTQPPSVSGAPGQRVTISCSGSSSNIGSNSVSWYQQL |
| (LC) | PGTAPKLLIYDNSKRPSGVPDRFSGSKSGTSASLAITGLQ |
| | SEDEADYYCQSRDTYGYYWVFGGGTKLTVLGQPKAAPSVT |
| | LFPPSSEELQANKATLVCLISDFYPGAVTVAWKGDSSPVK |
| | AGVETTTPSKQSNNKYAASSYLSLTPEQWKSHRSYSCQVT |
| | HEGSTVEKTVAPTECS (SEQ ID NO: 24) |

Some embodiments of the disclosure are anti-IGF-1R inhibitor mAbs or antigen binding fragments thereof, comprising a heavy chain comprising a variable heavy chain CDR1, a variable heavy chain CDR2, and a variable heavy chain CDR3, wherein the variable heavy chain CDR1 comprises an amino acid sequence SEQ ID NO:17, the variable heavy chain CDR2 comprises an amino acid sequence SEQ ID NO:18; and the variable heavy chain CDR3 comprises an amino acid sequence SEQ ID NO:19 or at least a CDR with at least 80% of sequence identity after optimal alignment with SEQ ID NO:17, SEQ ID NO:18, and SEQ ID NO:19.

The anti-IGF-1R inhibitor mAbs or antibody or antigen binding fragment thereof may additionally comprise a light chain which is paired with the heavy chain to form an antigen binding domain. In some embodiments, the light chain comprises a variable light chain CDR1, a variable light chain CDR2, and a variable light chain CDR3, wherein the variable light chain CDR1 comprises an amino acid sequence SEQ ID NO:20, the variable light chain CDR2 comprises an amino acid sequence SEQ ID NO:21; and the variable light chain CDR3 comprises an amino acid sequence SEQ ID NO:22 or at least a CDR with at least 80% of homology after optimal alignment with SEQ ID NO:20, SEQ ID NO:21, and SEQ ID NO:22.

In some embodiments, the anti-IGF-1R inhibitor mAbs or antigen binding fragment thereof comprises a heavy chain amino acid sequence of SEQ ID NO:23 or at least a heavy chain with at least 85%, 90%, 95%, 97%, 98%, or 99% of sequence identity after optimal alignment with SEQ ID NO:23. Alternatively, or in addition, the anti-IGF-1R inhibitor mAbs or antigen binding fragment thereof may comprise a light chain having an amino acid sequence of SEQ ID NO:24 or at least a heavy chain with at least 85%, 90%, 95%, 97%, 98%, or 99% of sequence identity after optimal alignment with SEQ ID NO:24.

### Example 4

### AVE1642

AVE1642 and other related IGF-1R inhibitor antibodies and their methods of preparation can be found in WO 2003/106621, which is hereby incorporated by reference in its entirety.

Heavy Chain CDRs-AVE1642

| HCDR1 | HCDR2 | HCDR3 |
|---|---|---|
| SYWMH | EINPSNGRTNYNEKFKR | GRPDYYGSSKWYFDV |
| (SEQ ID NO: 25) | (SEQ ID NO: 26) | (SEQ ID NO: 27) |
| GYTFTSYWMH | EINPSNGRTN | GRPDYYGSSKWYFDV |
| (SEQ ID NO: 75) | (SEQ ID NO: 76) | (SEQ ID NO: 27) |
| SYWMH | EINPSNGRTN | GRPDYYGSSKWYFDV |
| (SEQ ID NO: 25) | (SEQ ID NO: 76) | (SEQ ID NO: 27) |
| SYWMH | EINPSNGRTNYNQKFQG | GRPDYYGSSKWYFDV |
| (SEQ ID NO: 25) | (SEQ ID NO: 77) | (SEQ ID NO: 27) |
| GYTFTSYWMH | EINPSNGRTNYNQKFQG | GRPDYYGSSKWYFDV |
| (SEQ ID NO: 75) | (SEQ ID NO: 77) | (SEQ ID NO: 27) |

Light Chain CDRs-AVE1642

| LCDR1 | LCDR2 | LCDR3 |
|---|---|---|
| RSSQSIVHSNVNTYLE | KVSNRFS | FQGSHVPPT |
| (SEQ ID NO: 28) | (SEQ ID NO: 29) | (SEQ ID NO: 30) |

Variable Domains-AVE1642

| | |
|---|---|
| Heavy Chain | QVQLQQSGAELVKPGASVKLSCKASGYTFTSYWMHWVKQRPGQGLE |
| (VH1) | WIGEINPSNGRTNYNEKFKRKATLTVDKSSSTAYMQLSSLTSEDSA |
| | VYYFARGRPDYYGSSKWYFDVWGAGTTVTVSS (SEQ ID NO: 31) |
| Heavy Chain | QVQLVQSGAEVVKPGASVKLSCKASGYTFTSYWMHWVKQRPGQGLEW |
| (VH2) | IGEINPSNGRTNYNQKFQGKATLTVDKSSSTAYMQLSSLTSEDSAV |
| | YYFARGRPDYYGSSKWYFDVWGQGTTVTVSS (SEQ ID NO: 78) |
| Heavy Chain | QVQLVQSGAEVVKPGASVKLSCKASGYTFTSYWMHWVKQRPGQGLE |
| (VH3) | WIGEINPSNGRTNYNQKFQGKATLTVDKSSSTAYMQLSSLTSEDSAV |
| | YYFARGRPDYYGSSKWYFDVWGQGTTVTVS (SEQ ID NO: 79) |
| Light Chain | DVLMTQTPLSLPVSLGDQASISCRSSQSIVHSNVNTYLEWYLQKPGQS |
| (VL1) | PKLLIYKVSNRFSGVPDRESGSGSGTDFTLRISRVEAEDLGIYYCFQG |
| | SHVPPTFGGGTKLEIKR (SEQ ID NO: 32) |
| Light Chain | DVVMTQTPLSLPVSLGDPASISCRSSQSIVHSNVNTYLEWYLQKPGQS |
| (VL2) | PRLLIYKVSNRFSGVPDRESGSGAGTDFTLRISRVEAEDLGIYYCFQG |
| | SHVPPTFGGGTKLEIKR (SEQ ID NO: 80) |
| Light Chain | DVLMTQTPLSLPVSLGDPASISCRSSQSIVHSNVNTYLEWYLQKPGQS |
| (VL3) | PKLLIYKVSNRFSGVPDRESGSGAGTDFTLRISRVEAEDLGIYYCFQG |
| | SHVPPTFGGGTKLEIKR (SEQ ID NO: 81) |
| Light Chain | DVLMTQTPLSLPVSLGDPASISCRSSQSIVHSNVNTYLEWYLQKPGQS |
| (VL4) | PRLLIYKVSNRFSGVPDRESGSGAGTDFTLRISRVEAEDLGIYYCFQG |
| | SHVPPTFGGGTKLEIKR (SEQ ID NO: 82) |
| Light Chain | DVVMTQTPLSLPVSLGDPASISCRSSQSIVHSNVNTYLEWYLQKPGQS |
| (VL5) | PKLLIYKVSNRFSGVPDRESGSGAGTDFTLRISRVEAEDLGIYYCFQG |
| | SHVPPTFGGGTKLEIKR (SEQ ID NO: 83) |

Some embodiments of the disclosure are anti-IGF-1R inhibitor mAbs or antigen binding fragments thereof, comprising a heavy chain comprising a variable heavy chain CDR1, a variable heavy chain CDR2, and a variable heavy chain CDR3, wherein the variable heavy chain CDR1 comprises an amino acid sequence SEQ ID NO:25, the variable heavy chain CDR2 comprises an amino acid sequence SEQ ID NO:26; and the variable heavy chain CDR3 comprises an amino acid sequence SEQ ID NO:27 or at least a CDR with at least 80% of sequence identity after optimal alignment with SEQ ID NO:25, SEQ ID NO:26, and SEQ ID NO:27.

The anti-IGF-1R inhibitor mAbs or antibody or antigen binding fragment thereof may additionally comprise a light chain which is paired with the heavy chain to form an antigen binding domain. In some embodiments, the light chain comprises a variable light chain CDR1, a variable light chain CDR2, and a variable light chain CDR3, wherein the variable light chain CDR1 comprises an amino acid sequence SEQ ID NO:28, the variable light chain CDR2 comprises an amino acid sequence SEQ ID NO:29; and the variable light chain CDR3 comprises an amino acid sequence SEQ ID NO:30 or at least a CDR with at least 80% of homology after optimal alignment with SEQ ID NO:28, SEQ ID NO:29, and SEQ ID NO:30.

In some embodiments, the anti-IGF-1R inhibitor mAbs or antigen binding fragment thereof comprises a heavy chain amino acid sequence of SEQ ID NO:31 or at least a heavy chain with at least 85%, 90%, 95%, 97%, 98%, or 99% of sequence identity after optimal alignment with SEQ ID NOs:31, 78, or 79. Alternatively, or in addition, the anti-IGF-1R inhibitor mAbs or antigen binding fragment thereof may comprise a light chain having an amino acid sequence of SEQ ID NO:32 or at least a heavy chain with at least 85%, 90%, 95%, 97%, 98%, or 99% of sequence identity after optimal alignment with SEQ ID NOs:32, 80, 81, 82, or 83.

### Example 5

### Figitumumab

Figitumumab and other related IGF-1R inhibitor antibodies and their methods of preparation can be found in U.S. Pat. No. 7,037,498 which is hereby incorporated by reference in its entirety.

Heavy Chain CDRs-Figitumumab

| HCDR1 | HCDR2 | HCDR3 |
|---|---|---|
| GFTFSSYAMN | AISGSGGTTFYADSVKG | DLGWSDSYYYYYGMDV |
| (SEQ ID NO: 33) | (SEQ ID NO: 34) | (SEQ ID NO: 35) |

Light Chain CDRs-Figitumumab

| LCDR1 | LCDR2 | LCDR3 |
|---|---|---|
| RASQGIRNDLG | AASRLHR | LOHNSYPCS |
| (SEQ ID NO: 36) | (SEQ ID NO: 37) | (SEQ ID NO: 38) |

| | |
|---|---|
| Heavy Chain | EVQLLESGGGLVQPGGSLRLSCTASGFTFSSYAMNWVRQA |
| (HC) | PGKGLEWVSAISGSGGTTFYADSVKGRFTISRDNSRTTLY |
| | LQMNSLRAEDTAVYYCAKDLGWSDSYYYYYGMDVWGQGTT |
| | VTVSSASTKGPSVFPLAPCSRSTSESTAALGCLVKDYFPE |
| | PVTVSWNSGALTSGVHTFPAVLQSSGLYSLSSVVTVPSSN |
| | FGTQTYTCNVDHKPSNTKVDKTVERKCCVECPPCPAPPVA |
| | GPSVFLFPPKPKDTLMISRTPEVTCVVVDVSHEDPEVQFN |
| | WYVDGVEVHNAKTKPREEQFNSTFRVVSVLTVVHQDWLNG |
| | KEYKCKVSNKGLPAPIEKTISKTKGQPREPQVYTLPPSRE |
| | EMTKNQVSLTCLVKGFYPSDIAVEWESNGQPENNYKTTPP |
| | MLDSDGSFFLYSKLTVDKSRWQQGNVFSCSVMHEALHNHY |
| | TQKSLSLSPGK (SEQ ID NO: 39) |
| Light Chain | DIQMTQFPSSLSASVGDRVTITCRASQGIRNDLGWYQQKP |
| (LC) | GKAPKRLIYAASRLHRGVPSRFSGSGSGTEFTLTISSLQP |
| | EDFATYYCLQHNSYPCSFGQGTKLEIKRTVAAPSVFIFPP |
| | SDEQLKSGTASVVCLLNNFYPREAKVQWKVDNALQSGNSQ |
| | ESVTEQDSKDSTYSLSSTLTLSKADYEKHKVYACEVTHQG |
| | LSSPVTKSFNRGEC (SEQ ID NO: 40) |

Some embodiments of the disclosure are anti-IGF-1R inhibitor mAbs or antigen binding fragments thereof, comprising a heavy chain comprising a variable heavy chain CDR1, a variable heavy chain CDR2, and a variable heavy chain CDR3, wherein the variable heavy chain CDR1 comprises an amino acid sequence SEQ ID NO:33, the variable heavy chain CDR2 comprises an amino acid sequence SEQ ID NO:34; and the variable heavy chain CDR3 comprises an amino acid sequence SEQ ID NO:35 or at least a CDR with at least 80% of sequence identity after optimal alignment with SEQ ID NO:33, SEQ ID NO:34, and SEQ ID NO:35.

The anti-IGF-1R inhibitor mAbs or antibody or antigen binding fragment thereof may additionally comprise a light chain which is paired with the heavy chain to form an antigen binding domain. In some embodiments, the light chain comprises a variable light chain CDR1, a variable light chain CDR2, and a variable light chain CDR3, wherein the variable light chain CDR1 comprises an amino acid sequence SEQ ID NO:36, the variable light chain CDR2 comprises an amino acid sequence SEQ ID NO:37; and the variable light chain CDR3 comprises an amino acid sequence SEQ ID NO:38 or at least a CDR with at least 80% of homology after optimal alignment with SEQ ID NO:36, SEQ ID NO:37, and SEQ ID NO:38.

In some embodiments, the anti-IGF-1R inhibitor mAbs or antigen binding fragment thereof comprises a heavy chain amino acid sequence of SEQ ID NO:39 or at least a heavy chain with at least 85%, 90%, 95%, 97%, 98%, or 99% of sequence identity after optimal alignment with SEQ ID NO:39. Alternatively, or in addition, the anti-IGF-1R inhibitor mAbs or antigen binding fragment thereof may comprise a light chain having an amino acid sequence of SEQ ID NO:40 or at least a heavy chain with at least 85%, 90%, 95%, 97%, 98%, or 99% of sequence identity after optimal alignment with SEQ ID NO:40.

### Example 6

### Dusigitumab

Dusigitumab (MEDI-573) and other related IGF-1R inhibitor antibodies and their methods of preparation can be found in U.S. Pat. No. 7,939,637 which is hereby incorporated by reference in its entirety.

Heavy Chain CDRs-Dusigitumab

| HCDR1 | HCDR2 | HCDR3 |
|---|---|---|
| SYDIN | WMNPNSGNTGYAQKFQG | DPYYYYYGMDV |
| (SEQ ID NO: 41) | (SEQ ID NO: 42) | (SEQ ID NO: 43) |

Light Chain CDRs-Dusigitumab

| LCDR1 | LCDR2 | LCDR3 |
|---|---|---|
| SGSSSNIENNHVS | DNNKRPS | ETWDTSLSAGRV |
| (SEQ ID NO: 44) | (SEQ ID NO: 45) | (SEQ ID NO: 46) |

| | |
|---|---|
| Heavy Chain | QVQLVQSGAEVKKPGASVKVSCKAS |
| (HC) | GYTFTSYDINWVRQATGQGLEWMGW |
| | MNPNSGNTGYAQKFQGRVTMTRNTS |
| | ISTAYMELSSLRSEDTAVYYCARDP |
| | YYYYYGMDVWGQGTTVTVSSASTKG |
| | PSVFPLAPCSRSTSESTAALGCLVK |
| | DYFPEPVTVSWNSGALTSGVHTFPA |
| | VLQSSGLYSLSSVVTVPSSNFGTQT |
| | YTCNVDHKPSNTKVDKTVERKCCVE |
| | CPPCPAPPVAGPSVFLFPPKPKDTL |
| | MISRTPEVTCVVVDVSHEDPEVQFN |
| | WYVDGVEVHNAKTKPREEQFNSTFR |
| | VVSVLTVVHQDWLNGKEYKCKVSNK |
| | GLPAPIEKTISKTKGQPREPQVYTL |
| | PPSREEMTKNQVSLTCLVKGFYPSD |
| | IAVEWESNGQPENNYKTTPPMLDSD |
| | GSFFLYSKLTVDKSRWQQGNVFSCS |
| | VMHEALHNHYTQKSLSLSPGK |
| | (SEQ ID NO: 47) |
| Light Chain | QSVLTQPPSVSAAPGQKVTISCSGS |
| (LC) | SSNIENNHVSWYQQLPGTAPKLLIY |
| | DNNKRPSGIPDRFSGSKSGTSATLG |
| | ITGLQTGDEADYYCETWDTSLSAGR |
| | VFGGGTKLTVLGQPKAAPSVTLFPP |
| | SSEELQANKATLVCLISDFYPGAVT |
| | VAWKADSSPVKAGVETTTPSKQSNN |
| | KYAASSYLSLTPEQWKSHRSYSCQV |
| | THEGSTVEKTVAPTECS |
| | (SEQ ID NO: 48) |

Some embodiments of the disclosure are anti-IGF-1R inhibitor mAbs or antigen binding fragments thereof, comprising a heavy chain comprising a variable heavy chain CDR1, a variable heavy chain CDR2, and a variable heavy chain CDR3, wherein the variable heavy chain CDR1 comprises an amino acid sequence SEQ ID NO:41, the variable heavy chain CDR2 comprises an amino acid sequence SEQ ID NO:42; and the variable heavy chain CDR3 comprises an amino acid sequence SEQ ID NO:43 or at least a CDR with at least 80% of sequence identity after optimal alignment with SEQ ID NO:41, SEQ ID NO:42, and SEQ ID NO:43.

The anti-IGF-1R inhibitor mAbs or antibody or antigen binding fragment thereof may additionally comprise a light chain which is paired with the heavy chain to form an antigen binding domain. In some embodiments, the light chain comprises a variable light chain CDR1, a variable light chain CDR2, and a variable light chain CDR3, wherein the variable light chain CDR1 comprises an amino acid sequence SEQ ID NO:44, the variable light chain CDR2 comprises an amino acid sequence SEQ ID NO:45; and the variable light chain CDR3 comprises an amino acid sequence SEQ ID NO:46 or at least a CDR with at least 80% of homology after optimal alignment with SEQ ID NO:44, SEQ ID NO:45, and SEQ ID NO:46.

In some embodiments, the anti-IGF-1R inhibitor mAbs or antigen binding fragment thereof comprises a heavy chain amino acid sequence of SEQ ID NO:39 or at least a heavy chain with at least 85%, 90%, 95%, 97%, 98%, or 99% of sequence identity after optimal alignment with SEQ ID NO:47. Alternatively, or in addition, the anti-IGF-1R inhibitor mAbs or antigen binding fragment thereof may comprise a light chain having an amino acid sequence of SEQ ID NO:40 or at least a heavy chain with at least 85%, 90%, 95%, 97%, 98%, or 99% of sequence identity after optimal alignment with SEQ ID NO:48.

### Example 7

### Cixutumumab

Cixutumumab and other related IGF-1R inhibitor antibodies and their methods of preparation can be found in U.S. Pat. No. 7,638,605 which is hereby incorporated by reference in its entirety.

Heavy Chain CDRs-Cixutumumab

| HCDR1 | HCDR2 | HCDR3 |
|---|---|---|
| SYAIS | GIIPIFGTANYAQKFQ | APLRFLEWSTQDHYYYYYMDV |
| (SEQ ID NO: 49) | (SEQ ID NO: 50) | (SEQ ID NO: 51) |

Light Chain CDRs-Cixutumumab

| LCDR1 | LCDR2 | LCDR3 |
|---|---|---|
| QGDSLRSYYAT | GENKRPS | KSRDGSGQHLV |
| (SEQ ID NO: 52) | (SEQ ID NO: 53) | (SEQ ID NO: 54) |

| | |
|---|---|
| Heavy Chain | EVQLVQSGAEVKKPGSSVKVSCKASGGTFSSYAISWVRQA |
| (HC) | PGQGLEWMGGIIPIFGTANYAQKFQGRVTITADKSTSTAY |
| | MELSSLRSEDTAVYYCARAPLRFLEWSTQDHYYYYYMDVW |
| | GKGTTVTVSSASTKGPSVFPLAPSSKSTSGGTAALGCLVK |
| | DYFPEPVTVSWNSGALTSGVHTFPAVLQSSGLYSLSSVVT |
| | VPSSSLGTQTYICNVNHKPSNTKVDKKVEPKSCDKTHTCP |
| | PCPAPELLGGPSVFLFPPKPKDTLMISRTPEVTCVVVDVS |
| | HEDPEVKFNWYVDGVEVHNAKTKPREEQYNSTYRVVSVLT |
| | VLHQDWLNGKEYKCKVSNKALPAPIEKTISKAKGQPREPQ |
| | VYTLPPSREEMTKNQVSLTCLVKGFYPSDIAVEWESNGQP |
| | ENNYKTTPPVLDSDGSFFLYSKLTVDKSRWQQGNVFSCSV |
| | MHEALHNHYTQKSLSLSPGK (SEQ ID NO: 55) |
| Light Chain | SSELTQDPAVSVALGQTVRITCQGDSLRSYYATWYQQKPG |
| (LC) | QAPILVIYGENKRPSGIPDRFSGSSSGNTASLTITGAQAE |
| | DEADYYCKSRDGSGQHLVFGGGTKLTVLGQPKAAPSVTLF |
| | PPSSEELQANKATLVCLISDFYPGAVTVAWKADSSPVKAG |
| | VETTTPSKQSNNKYAASSYLSLTPEQWKSHRSYSCQVTHE |
| | GSTVEKTVAPAECS |
| | (SEQ ID NO: 56) |

Some embodiments of the disclosure are anti-IGF-1R inhibitor mAbs or antigen binding fragments thereof, comprising a heavy chain comprising a variable heavy chain CDR1, a variable heavy chain CDR2, and a variable heavy chain CDR3, wherein the variable heavy chain CDR1 comprises an amino acid sequence SEQ ID NO:49, the variable heavy chain CDR2 comprises an amino acid sequence SEQ ID NO:50; and the variable heavy chain CDR3 comprises an amino acid sequence SEQ ID NO:51 or at least a CDR with at least 80% of sequence identity after optimal alignment with SEQ ID NO:49, SEQ ID NO:50, and SEQ ID NO:51.

The anti-IGF-1R inhibitor mAbs or antibody or antigen binding fragment thereof may additionally comprise a light chain which is paired with the heavy chain to form an antigen binding domain. In some embodiments, the light chain comprises a variable light chain CDR1, a variable light chain CDR2, and a variable light chain CDR3, wherein the variable light chain CDR1 comprises an amino acid sequence SEQ ID NO:52, the variable light chain CDR2 comprises an amino acid sequence SEQ ID NO:53; and the variable light chain CDR3 comprises an amino acid sequence SEQ ID NO:54 or at least a CDR with at least 80% of homology after optimal alignment with SEQ ID NO:52, SEQ ID NO:53, and SEQ ID NO:54.

In some embodiments, the anti-IGF-1R inhibitor mAbs or antigen binding fragment thereof comprises a heavy chain amino acid sequence of SEQ ID NO:55 or at least a heavy chain with at least 85%, 90%, 95%, 97%, 98%, or 99% of sequence identity after optimal alignment with SEQ ID NO:55. Alternatively, or in addition, the anti-IGF-1R inhibitor mAbs or antigen binding fragment thereof may comprise a light chain having an amino acid sequence of SEQ ID NO:56 or at least a heavy chain with at least 85%, 90%, 95%, 97%, 98%, or 99% of sequence identity after optimal alignment with SEQ ID NO:56.

### Example 8

### BIIB022

BIIB022 and other related IGF-1R inhibitor antibodies and their methods of preparation can be found in U.S. Pat. No. 7,612,178 which is hereby incorporated by reference in its entirety

Heavy Chain CDRs-BIIB022

| HCDR1 | HCDR2 | HCDR3 |
|---|---|---|
| IYRMQ | GISPSGGTTWYADSVKG | WSGGSGYAFDI (SEQ |
| (SEQ ID NO: 57) | (SEQ ID NO: 58) | ID NO: 59) |

Light Chain CDRs-BIIB022

| LCDR1 | LCDR2 | LCDR3 |
|---|---|---|
| QASRDIRNYN | DASSLQT | QQFDSLPHT |
| (SEQ ID NO: 60) | (SEQ ID NO: 61) | (SEQ ID NO: 62) |

| | |
|---|---|
| Heavy Chain | EVQLLESGGGLVQPGGSLRLSCAASGFTFSIYRMQWVRQAPGKGLEW |
| (HC) | VSGISPSGGTTWYADSVKGRFTISRDNSKNTLYLQMNSLRAEDTAVY |
| | YCARWSGGSGYAFDIWGQGTMVTVSS (SEQ ID NO: 63) |
| Light Chain | DIQMTQSPLSLSASVGDRVTITCQASRDIRNYLNWYQQKPGKAPKLLI |
| (LC) | YDASSLQTGVPSRFGGSGSGTDFSFTIGSLQPEDIATYYCQQFDSLPH |
| | TFGQGTKLEIK (SEQ ID NO: 64) |

Some embodiments of the disclosure are anti-IGF-1R inhibitor mAbs or antigen binding fragments thereof, comprising a heavy chain comprising a variable heavy chain CDR1, a variable heavy chain CDR2, and a variable heavy chain CDR3, wherein the variable heavy chain CDR1 comprises an amino acid sequence SEQ ID NO:57, the variable heavy chain CDR2 comprises an amino acid sequence SEQ ID NO:58; and the variable heavy chain CDR3 comprises an amino acid sequence SEQ ID NO:59 or at least a CDR with at least 80% of sequence identity after optimal alignment with SEQ ID NO:57, SEQ ID NO:58, and SEQ ID NO:59.

The anti-IGF-1R inhibitor mAbs or antibody or antigen binding fragment thereof may additionally comprise a light chain which is paired with the heavy chain to form an antigen binding domain. In some embodiments, the light chain comprises a variable light chain CDR1, a variable light chain CDR2, and a variable light chain CDR3, wherein the variable light chain CDR1 comprises an amino acid sequence SEQ ID NO:60, the variable light chain CDR2 comprises an amino acid sequence SEQ ID NO:61; and the variable light chain CDR3 comprises an amino acid sequence SEQ ID NO:62 or at least a CDR with at least 80% of homology after optimal alignment with SEQ ID NO:60, SEQ ID NO:61, and SEQ ID NO:62.

In some embodiments, the anti-IGF-IR inhibitor mAbs or antigen binding fragment thereof comprises a heavy chain amino acid sequence of SEQ ID NO:63 or at least a heavy chain with at least 85%, 90%, 95%, 97%, 98%, or 99% of sequence identity after optimal alignment with SEQ ID NO:63. Alternatively, or in addition, the anti-IGF-IR inhibitor mAbs or antigen binding fragment thereof may comprise a light chain having an amino acid sequence of SEQ ID NO:64 or at least a heavy chain with at least 85%, 90%, 95%, 97%, 98%, or 99% of sequence identity after optimal alignment with SEQ ID NO:64.

### Example 9

### Robatumumab

Heavy Chain (HC) and Light Chain (LC) for Robatumumab

| | |
|---|---|
| Heavy Chain | EVQLVQSGGG LVKPGGSLRL SCAASGFTFS SFAMHWVRQA |
| (HC) | PGKGLEWISV IDTRGATYYADSVKGRFTIS RDNAKNSLYL |
| | QMNSLRAEDT AVYYCARLGN FYYGMDVWGQ GTTVTVSSAS |
| | TKGPSVFPLA PSSKSTSGGT AALGCLVKDY FPEPVTVSWN |
| | SGALTSGVHT FPAVLQSSGLYSLSSVVTVP SSSLGTQTYI |
| | CNVNHKPSNT KVDKKVEPKS CDKTHTCPPC PAPELLGGPS |
| | VFLFPPKPKD TLMISRTPEV TCVVVDVSHE DPEVKFNWYV |
| | DGVEVHNAKT KPREEQYNSTYRVVSVLTVL HQDWLNGKEY |
| | KCKVSNKALP APIEKTISKA KGQPREPQVY TLPPSRDELT |
| | KNQVSLTCLV KGFYPSDIAV EWESNGQPEN NYKTTPPVLD |
| | SDGSFFLYSK LTVDKSRWQQGNVFSCSVMH EALHNHYTQK |
| | SLSLSPGK (SEQ ID NO: 65) |
| Light Chain | EIVLTQSPGTLSVSPGERATLSCRASQSIGSSLHWYQQKPGQA |
| (LC) | PRLLIKYASQSLSGIPDRESGSGSGTDFTLTISRLEPEDFAVY |
| | YCHQSSRLPHTFGQGTKVEIKRTVAAPSVFIFPPSDEQLKSGT |
| | ASVVCLLNNFYPREAKVQWKVDNALQSGNSQESVTEQDSKDST |
| | YSLSSTLTLSKADYEKHKVYACEVTHQGLSSPVTKSFNRGEC |
| | (SEQ ID NO: 66) |

In some embodiments, the anti-IGF-IR inhibitor mAbs or antigen binding fragment thereof comprises a heavy chain amino acid sequence of SEQ ID NO:65 or at least a heavy chain with at least 85%, 90%, 95%, 97%, 98%, or 99% of sequence identity after optimal alignment with SEQ ID NO:65. Alternatively, or in addition, the anti-IGF-IR inhibitor mAbs or antigen binding fragment thereof may comprise a light chain having an amino acid sequence of SEQ ID NO:66 or at least a heavy chain with at least 85%, 90%, 95%, 97%, 98%, or 99% of sequence identity after optimal alignment with SEQ ID NO:66.

In some embodiments, said IGF-1R inhibitor is a small molecule.

### Example 10

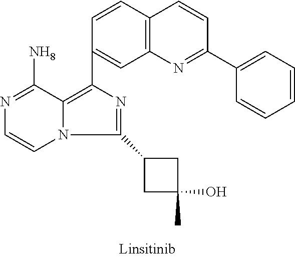 [SMILES: C[C@]1(O)C[C@@H](c2nc(-c3ccc4ccc(-c5ccccc5)nc4c3)c3c(N)nccn32)C1]

Linsitinib and other related IGF-1R inhibitor small molecules and their methods of preparation can be found in U.S. Pat. No. 8,101,613, which is hereby incorporated by reference in its entirety. Linsitinib and the other IGF-1R inhibitors described therein are predicted to have activity in the activity measures or assessments for the treatment of TED described herein.

### Example 11

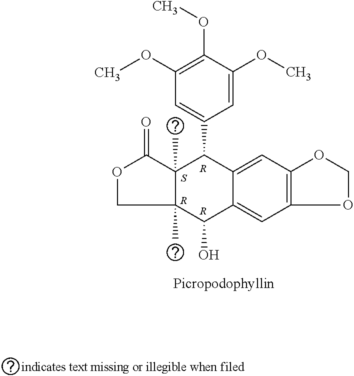

Picropodophyllin (AXL1717) and other related IGF-1R inhibitor small molecules and their methods of preparation can be found in US U.S. Pat. No. 4,567,253, which is hereby incorporated by reference in its entirety. Picropodophyllin and the other IGF-1R inhibitors described therein are predicted to have activity in the activity measures or assessments for the treatment of TED described herein.

### Example 12

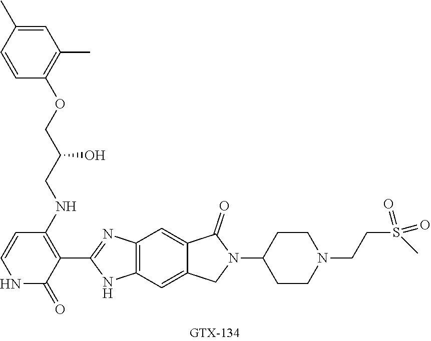 [SMILES: Cc1ccc(OC[C@H](O)CNc2cc[nH]c(=O)c2-c2nc3cc4c(cc3[nH]2)CN(C2CCN(CCS(C)(=O)=O)CC2)C4=O)c(C)c1]

GTX-134 and other related IGF-1R inhibitor small molecules and their methods of preparation can be found in U.S. Pat. No. 8,063,225, which is hereby incorporated by reference in its entirety. GTX-134 and the other IGF-1R inhibitors described therein are predicted to have activity in the activity measures or assessments for the treatment of TED described herein.

### Example 13

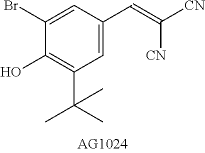

AG1024 and other related IGF-1R inhibitor small molecules and their methods of preparation can be found in WO1995024190, which is hereby incorporated by reference in its entirety. AG1024 and the other IGF-1R inhibitors described therein are predicted to have activity in the activity measures or assessments for the treatment of TED described herein.

### Example 14

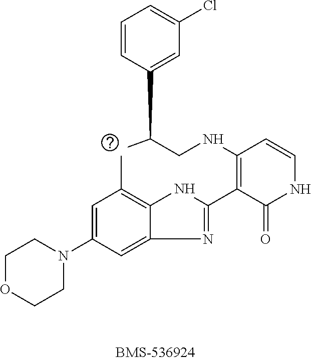 [SMILES: Cc1cc(N2CCOCC2)cc2nc(-c3c(NC[C@@H](C)c4cccc(Cl)c4)cc[nH]c3=O)[nH]c12]

BMS-536924 and other related IGF-1R inhibitor small molecules and their methods of preparation can be found in U.S. Pat. No. 7,081,454, which is hereby incorporated by reference in its entirety. BMS-536924 and the other IGF-1R inhibitors described therein are predicted to have activity in the activity measures or assessments for the treatment of TED described herein.

### Example 15

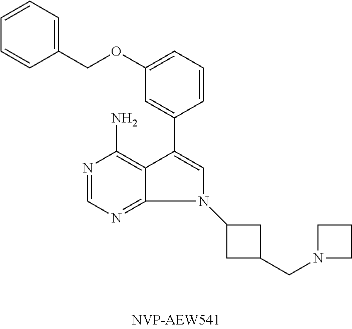 [SMILES: Nc1ncnc2c1c(-c1cccc(OCc3ccccc3)c1)cn2C1CC(CN2CCC2)C1]

NVP-AEW541 and other related IGF-1R inhibitor small molecules and their methods of preparation can be found in U.S. Pat. No. 7,326,699, which is hereby incorporated by reference in its entirety. NVP-AEW541 and the other IGF-1R inhibitors described therein are predicted to have activity in the activity measures or assessments for the treatment of TED described herein.

### Example 16

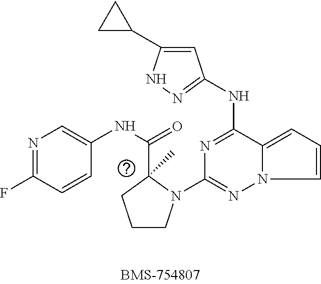 [SMILES: C[C@@]1(C(=O)Nc2ccc(F)nc2)CCCN1c1nc(Nc2cc(C3CC3)[nH]n2)c2cccn2n1]

BMS-754807 and other related IGF-1R inhibitor small molecules and their methods of preparation can be found in U.S. Pat. No. 7,534,792, which is hereby incorporated by reference in its entirety. BMS-754807 and the other IGF-1R inhibitors described therein are predicted to have activity in the activity measures or assessments for the treatment of TED described herein.

### Example 17

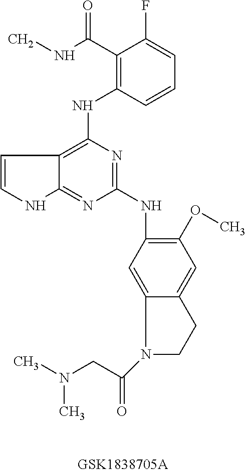 [SMILES: CNC(=O)c1c(F)cccc1Nc1nc(Nc2cc3c(cc2OC)CCN3C(=O)CN(C)C)nc2[nH]ccc12]

GSK1838705A and other related IGF-1R inhibitor small molecules and their methods of preparation can be found in U.S. Pat. No. 7,981,903, which is hereby incorporated by reference in its entirety. GSK1838705A and the other IGF-1R inhibitors described therein are predicted to have activity in the activity measures or assessments for the treatment of TED described herein.

### Example 18

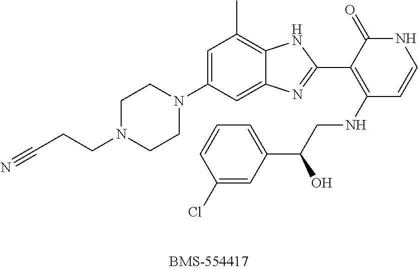 [SMILES: Cc1cc(N2CCN(CCC#N)CC2)cc2nc(-c3c(NC[C@@H](O)c4cccc(Cl)c4)cc[nH]c3=O)[nH]c12]

BMS-554417 and other related IGF-1R inhibitor small molecules and their methods of preparation can be found in U.S. Pat. No. 7,081,454, which is hereby incorporated by reference in its entirety. BMS-554417 and the other IGF-1R inhibitors described therein are predicted to have activity in the activity measures or assessments for the treatment of TED described herein.

### Example 19

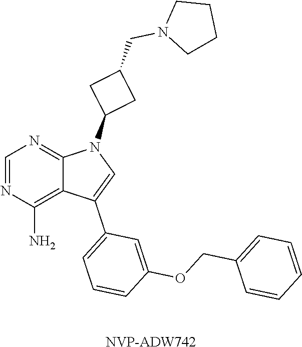 [SMILES: Nc1ncnc2c1c(-c1cccc(OCc3ccccc3)c1)cn2[C@H]1C[C@H](CN2CCCC2)C1 ; "NVP-ADW742"]

NVP-ADW742 and other related IGF-1R inhibitor small molecules and their methods of preparation can be found in U.S. Pat. No. 7,326,699, which is hereby incorporated by reference in its entirety. NVP-ADW742 and the other IGF-1R inhibitors described therein are predicted to have activity in the activity measures or assessments for the treatment of TED described herein.

### Example 20

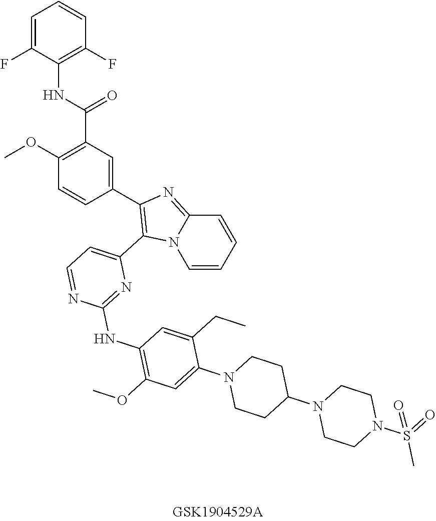 [SMILES: CCc1cc(Nc2nccc(-c3c(-c4ccc(OC)c(C(=O)Nc5c(F)cccc5F)c4)nc4ccccn34)n2)c(OC)cc1N1CCC(N2CCN(S(C)(=O)=O)CC2)CC1]

GSK1904529A and other related IGF-1R inhibitor small molecules and their methods of preparation can be found in U.S. Pat. No. 8,093,239, which is hereby incorporated by reference in its entirety. GSK1904529A and the other IGF-1R inhibitors described therein are predicted to have activity in the activity measures or assessments for the treatment of TED described herein.

### Example 21

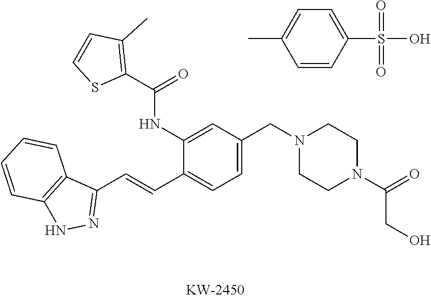 [SMILES: Cc1ccsc1C(=O)Nc1cc(CN2CCN(C(=O)CO)CC2)ccc1/C=C/c1n[nH]c2ccccc12 ; Cc1ccc(S(=O)(=O)O)cc1]

KW-2450, shown above as the tosylate salt but not limited thereto, and other related IGF-1R inhibitor small molecules and their methods of preparation can be found in WO2006080450, U.S. Pat. No. 7,605,272, and WO2011158931, which are hereby incorporated by reference in their entireties. KW-2450 and the other IGF-1R inhibitors described herein are predicted to have activity in the activity measures or assessments for the treatment of TED described herein.

### Example 22

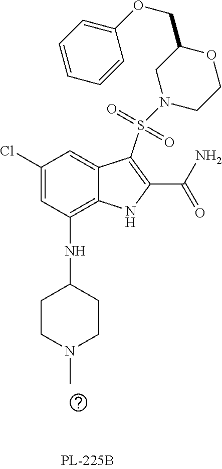 [SMILES: CN1CCC(Nc2cc(Cl)cc3c(S(=O)(=O)N4CCO[C@H](COc5ccccc5)C4)c(C(N)=O)[nH]c23)CC1]

PL-225B and other related IGF-1R inhibitor small molecules and their methods of preparation can be found in WO2012145471 and WO2012007926, which is hereby incorporated by reference in its entirety. PL225B selectively inhibits IGF-1 R, resulting in inhibition of tumor cell proliferation and the induction of tumor cell apoptosis in IGF-1 R-overexpressing tumor cells. PL-225B and the other IGF-1R inhibitors described herein are predicted to have activity in the activity measures or assessments for the treatment of TED described herein.

### Example 23

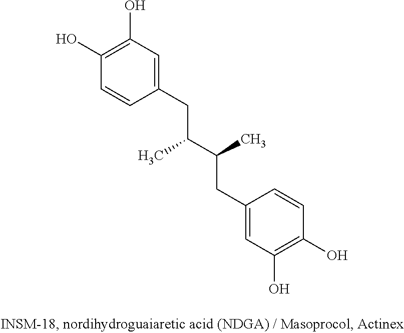 [SMILES: C[C@H](Cc1ccc(O)c(O)c1)[C@@H](C)Cc1ccc(O)c(O)c1]

INSM-18, nordihydroguaiaretic acid (NDGA) (shown above with relative stereochemistry, in which case it is also referred to as Masoprocol or Actinex, but not limited thereto) referred to in this Example as INSM-18, and other related IGF-1R inhibitor small molecules and their methods of preparation can be found at least in U.S. Pat. No. 2,373,192, which is hereby incorporated by reference in its entirety. INSM-18 directly inhibits activation of IGF-1 R and the c-erbB2/HER2/neu receptor, resulting in decreased proliferation of susceptible tumor cell populations. INSM-18 and the other IGF-1R inhibitors described herein are predicted to have activity in the activity measures or assessments for the treatment of TED described herein.

### Example 24

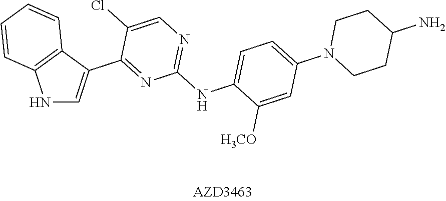 [SMILES: COc1cc(N2CCC(N)CC2)ccc1Nc1ncc(Cl)c(-c2c[nH]c3ccccc23)n1]

AZD3463 and other related IGF-1R inhibitor small molecules and their methods of preparation can be found in U.S. Pat. No. 8,461,170, which is hereby incorporated by reference in its entirety. AZD3463 is a potent ALK/IGF-1 R inhibitor, resulting in inhibition of neuroblastoma growth by overcoming crizotinib resistance and inducing apoptosis. AZD3463 and the other IGF-1R inhibitors described herein are predicted to have activity in the activity measures or assessments for the treatment of TED described herein.

### Example 25

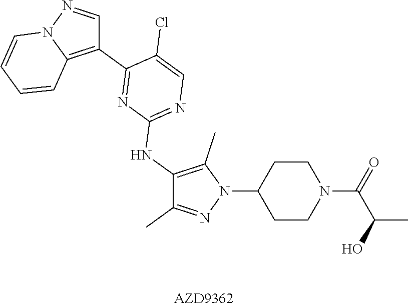 [SMILES: Cc1nn(C2CCN(C(=O)[C@@H](C)O)CC2)c(C)c1Nc1ncc(Cl)c(-c2cnn3ccccc23)n1]

AZD9362 and other related IGF-1R inhibitor small molecules and their methods of preparation can be found in Degorce, S L et al., “Discovery of a Potent, Selective, Orally Bioavailable, and Efficacious Novel 2-(Pyrazol-4-ylamino)-pyrimidine Inhibitor of the Insulin-like Growth Factor-1 Receptor (IGF-1R),” J Med Chem (2016), 59(10), 4859-4866., which is hereby incorporated by reference in its entirety. AZD9362 is a dual inhibitor of IGF-1R/InsR. AZD9362 and the other IGF-1R inhibitors described herein are predicted to have activity in the activity measures or assessments for the treatment of TED described herein.

### Example 26

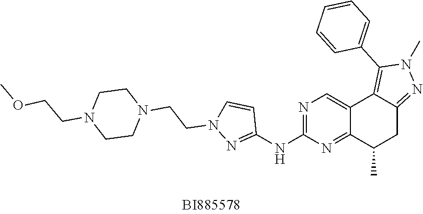

BI885578 and other related IGF-1R inhibitor small molecules and their methods of preparation can be found in U.S. Ser. No. 10/414,769, U.S. Pat. No. 9,150,578, and Sanderson M P et al., “BI 885578, a Novel IGF1R/INSR Tyrosine Kinase Inhibitor with Pharmacokinetic Properties That Dissociate Antitumor Efficacy and Perturbation of Glucose Homeostasis,” Mol Cancer Ther 2015 December; 14(12):2762-72, which are hereby incorporated by reference in its entirety. BI885578 is an IGF1R/INSR tyrosine kinase inhibitor distinguished by rapid intestinal absorption and a short in vivo half-life as a result of rapid metabolic clearance, resulting in inhibition of cell proliferation and induction of apoptosis in tumors. BI885578 and the other IGF-1R inhibitors described herein are predicted to have activity in the activity measures or assessments for the treatment of TED described herein.

### Example 27

### BI893923

BI893923 and other related IGF-1R inhibitor small molecules and their methods of preparation can be found in U.S. Pat. No. 8,546,443 and Titze M I et al., “An allometric pharmacokinetic/pharmacodynamics model for BI893923, a novel IGF-1 receptor inhibitor,” Cancer Chemother Pharmacol 2017 March; 79(3):545-558, which is hereby incorporated by reference in its entirety. BI893923 is an IGF1R/INSR tyrosine kinase inhibitor demonstrating anti-tumor efficacy and good tolerability. BI893923 and the other IGF-1R inhibitors described herein are predicted to have activity in the activity measures or assessments for the treatment of TED described herein.

### Example 28

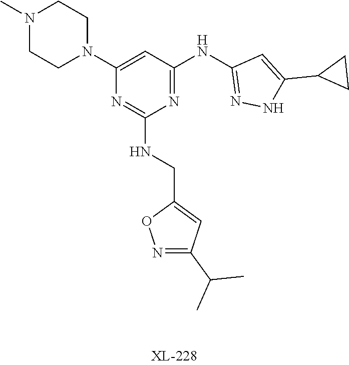 [SMILES: CC(C)c1cc(CNc2nc(Nc3cc(C4CC4)[nH]n3)cc(N3CCN(C)CC3)n2)on1]

XL-228 and other related IGF-1R inhibitor small molecules and their methods of preparation can be found in US20090232828, which is hereby incorporated by reference in its entirety. XL-228 is a broad protein kinase inhibitor that contributes to cell proliferation, cell survival, and resistance to cytotoxic agents. XL-228 and the other IGF-1R inhibitors described herein are predicted to have activity in the activity measures or assessments for the treatment of TED described herein.

### Example 29

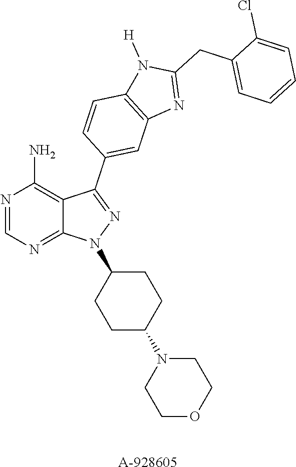 [SMILES: Nc1ncnc2c1c(-c1ccc3[nH]c(Cc4ccccc4Cl)nc3c1)nn2[C@H]1CC[C@H](N2CCOCC2)CC1 ; "A-928605"]

A-928605 and other related IGF-1R inhibitor small molecules and their methods of preparation can be found in U.S. Pat. No. 7,772,231 and WO2007079164, which are hereby incorporated by reference in its entirety. A-928605 is a potent inhibitor of IGF-IR both on the purified enzyme and intracellular IGF-IR phosphorylation. A-928605 and the other IGF-1R inhibitors described herein are predicted to have activity in the activity measures or assessments for the treatment of TED described herein.

### Example 30

### Istiratumab (MM-141)

Istiratumab and other related IGF-1R inhibitor antibodies and their methods of preparation can be found in U.S. Pat. No. 8,476,409, which is hereby incorporated by reference in its entirety.

Heavy Chain CDRs-Istiratumab

| HCDR1 | HCDR2 | HCDR3 |
|---|---|---|
| GFMFSRYPMH | ISGSGGATPYADSVKG | DFYQILTGNAFDY |
| (SEQ ID NO: 67) | (SEQ ID NO: 68) | (SEQ ID NO: 69) |

Light Chain CDRs-Istiratumab

| LCDR1 | LCDR2 | LCDR3 |
|---|---|---|
| RASQGISSYLA | AKSTLQS | QQYWTFPLT |
| (SEQ ID NO: 70) | (SEQ ID NO: 71) | (SEQ ID NO: 72) |

| | |
|---|---|
| Heavy Chain | EVQLLQSGGGLVQPGGSLRLSCAASGFMFS |
| (HC) | RYPMHWVRQAPGKGLEWVGSISGSGGATPY |
| | ADSVKGRFTISRDNSKNTLYLQMNSLRAED |
| | TAVYYCAKDFYQILTGNAFDYWGQGTTVTV |
| | SSASTKGPSVFPLAPSSKSTSGGTAALGCL |
| | VKDYFPEPVTVSWNSGALTSGVHTFPAVLQ |
| | SSGLYSLSSVVTVPSSSLGTQTYICNVNHK |
| | PSNTKVDKKVEPKSCDKTHTCPPCPAPELL |
| | GGPSVFLFPPKPKDTLMISRTPEVTCVVVD |
| | VSHEDPEVKFNWYVDGVEVHNAKTKPREEQ |
| | YNSTYRVVSVLTVLHQDWLNGKEYKCKVSN |
| | KALPAPIEKTISKAKGQPREPQVYTLPPSR |
| | EEMTKNQVSLTCLVKGFYPSDIAVEWESNG |
| | QPENNYKTTPPVLDSDGSFFLYSKLTVDKS |
| | RWQQGNVFSCSVMHEALHNHYTQKSLSLSP |
| | GGGGGSGGGGSGGGGSQVQLVQSGGGLVQP |
| | GGSLRLSCAASGFTFDDYAMHWVRQAPGKG |
| | LEWVAGISWDSGSTGYADSVKGRFTISRDN |
| | AKNSLYLQMNSLRAEDTALYYCARDLGAYQ |
| | WVEGFDYWGQGTLVTVSSASTGGGGSGGGG |
| | SGGGGSGGGGSSYELTQDPAVSVALGQTVR |
| | ITCQGDSLRSYYASWYQQKPGQAPVLVIYG |
| | KNNRPSGIPDRESGSTSGNSASLTITGAQA |
| | EDEADYYCNSRDSPGNQWVFGGGTKVTVLG |
| | (SEQ ID NO: 73) |
| Light Chain | DIQMTQSPSSLSASLGDRVTITCRASQGIS |
| (LC) | SYLAWYQQKPGKAPKLLIYAKSTLQSGVPS |
| | RFSGSGSGTDFTLTISSLQPEDSATYYCQQ |
| | YWTFPLTFGGGTKVEIKRTVAAPSVFIFPP |
| | SDEQLKSGTASVVCLLNNFYPREAKVQWKV |
| | DNALQSGNSQESVTEQDSKDSTYSLSSTLT |
| | LSKADYEKHKVYACEVTHQGLSSPVTKSFN |
| | RGEC |
| | (SEQ ID NO: 74) |

Some embodiments of the disclosure are anti-IGF-1R inhibitor mAbs or antigen binding fragments thereof, comprising a heavy chain comprising a variable heavy chain CDR1, a variable heavy chain CDR2, and a variable heavy chain CDR3, wherein the variable heavy chain CDR1 comprises an amino acid sequence SEQ ID NO:67, the variable heavy chain CDR2 comprises an amino acid sequence SEQ ID NO:68; and the variable heavy chain CDR3 comprises an amino acid sequence SEQ ID NO:69 or at least a CDR with at least 80% of sequence identity after optimal alignment with SEQ ID NO:67, SEQ ID NO:68, and SEQ ID NO:69.

The anti-IGF-1R inhibitor mAbs or antibody or antigen binding fragment thereof may additionally comprise a light chain which is paired with the heavy chain to form an antigen binding domain. In some embodiments, the light chain comprises a variable light chain CDR1, a variable light chain CDR2, and a variable light chain CDR3, wherein the variable light chain CDR1 comprises an amino acid sequence SEQ ID NO:70, the variable light chain CDR2 comprises an amino acid sequence SEQ ID NO:71; and the variable light chain CDR3 comprises an amino acid sequence SEQ ID NO:72 or at least a CDR with at least 80% of homology after optimal alignment with SEQ ID NO:70, SEQ ID NO:71, and SEQ ID NO:72.

In some embodiments, the anti-IGF-1R inhibitor mAbs or antigen binding fragment thereof comprises a heavy chain amino acid sequence of SEQ ID NO:73 or at least a heavy chain with at least 85%, 90%, 95%, 97%, 98%, or 99% of sequence identity after optimal alignment with SEQ ID NO:73. Alternatively, or in addition, the anti-IGF-1R inhibitor mAbs or antigen binding fragment thereof may comprise a light chain having an amino acid sequence of SEQ ID NO:74 or at least a heavy chain with at least 85%, 90%, 95%, 97%, 98%, or 99% of sequence identity after optimal alignment with SEQ ID NO:74.

### Example 31

### Description of Randomized, Double-Masked, Placebo- and/or Active-Controlled, Parallel-Group, Multicenter Study Evaluating IGF-1R Inhibition in Subjects with Chronic/Inactive Thyroid Eye Disease (TED)

Overview. Multicenter, optionally double-masked, randomized, parallel-group, placebo- and/or active- (e.g., teprotumumab-) controlled clinical trials may be conducted to determine the efficacy and safety of any Study Drug disclosed herein in patients with either active/acute, or inactive/chronic, moderate-to-severe TED. The study may be conducted in male and non-pregnant female patients between the ages of 18 and 80 years, inclusive. Patients will be enrolled and randomly assigned on Day 1 in an appropriate ratio, (e.g., 1:1, 2:1, or 3:1) to receive placebo or active control or Study Drug administered as described herein. Subjects will be screened for the study within 4 weeks prior to the Baseline (Day 1) Visit. Subjects may be stratified by duration of disease, ≤2 years or >2 years.

Patient Population. The study may be designed to assess activity and safety in either 1) patients with moderate-to-severe active/acute TED or 2) moderate-to-severe inactive/chronic TED. Moderate to severe acute disease may be defined as: i) ≥3 mm proptosis beyond race/gender norms or beyond patient's pre-TED, ii) clinical activity score of at least 3, and iii) within 15 months of symptom onset. Moderate to severe chronic disease may be defined as: i) ≥3 mm proptosis beyond race/gender norms or beyond patient's pre-TED, ii) clinical activity score of 0 or 1, and iii) no significant progression or inflammatory symptoms within 1 year.

Treatment Period. The planned duration of the Treatment Period may be, e.g., 12, 24, or 48 weeks (3, 6, or 12 months), with an optional open-label extension study period. At the End of the Treatment Period—Week 12, Week 24, or week 48, as appropriate—primary endpoint responders, as well as non-responders who choose not to enroll in the open-label extension study, or will enter a safety Follow-Up Period. Subjects who are considered non-responders at the end of the treatment period may enroll in the open-label extension study.

All subjects will enter a Treatment Period of, e.g., 12-week or 24-week or 48-week. All study drug dosing or initial study drug dosing will be performed at the clinic under the supervision of clinic staff. On each dosing day, scheduled assessments (except for AE and concomitant medication use monitoring, which will be monitored throughout the clinic visit) will be completed prior to study drug dosing. Additional phone/email contacts and clinic visits may also be conducted for any subject experiencing a drug-related adverse event.

Study Endpoints. The primary end point may be either proptosis or diplopia, e.g. in the study eye. Proptosis may be assessed either as proportion of responders (where a responder is defined as a patient experiencing a ≥2 mm reduction from Baseline in the study eye without deterioration—≥2 mm increase—of proptosis in the fellow eye) or assessed as a continuous variable (i.e., average or median change from baseline), measured using a standardized exophthalmometer (e.g., Hertel). Diplopia may be assessed using any of the subject Gorman scale, the Goldman perimeter, or the cervical range of motion method, as long as the same assessment is used for all patients.

Secondary end points, measured as continuous variables, may include: proptosis, diplopia, orbital pain, MDI and PVR for inferior rectus, superior rectus, the medial rectus, lateral rectus and orbital fat, clinical activity score (CAS), circumference of calf and area of the lesion, inflammatory and fibrotic biomarkers, transcriptomics associated with IGF-1R inhibition, and results on the Graves' ophthalmopathy-specific quality-of-life questionnaire (GO-QoL) or appearance and functioning subscales thereof. Adverse events will also be assessed.

Subjects who prematurely discontinue study drug dosing will return to the clinic and undergo the scheduled End of Treatment Period assessments and will be encouraged to remain in the study and participate in the Follow-Up Period.

Inclusion Criteria. Major inclusion criteria will include the following:
Written informed consent.
Male or female subject between the ages of 18 and 80 years, inclusive, at Screening.
For chronic/inactive TED:
   Moderate-to-severe chronic/inactive TED (not sight-threatening but has an appreciable impact on daily life), usually associated with one or more of the following: lid retraction >2 mm, moderate or severe soft tissue involvement, and/or inconstant or constant diplopia.
   Initial diagnosis of TED >2 years prior to Screening. Clinical diagnosis of stable, chronic/inactive TED as determined by patient medical records indicating a CAS ≤1 in both eyes for at least 1 year prior to Screening or all of the following: (a) no progression in proptosis for at least 1 year prior to Screening; (b) if subject has history of diplopia due to TED, no progression in diplopia for at least 1 year prior to Screening; (c) no inflammatory symptoms for at least 1 year prior to Screening; and no new TED symptoms for at least 1 year prior to Screening.
   CAS ≤1 at Screening and Baseline visits.
For acute/active TED:
   Moderate-to-severe active TED (not sight-threatening but has an appreciable impact on daily life), usually associated with one or more of the following: lid retraction ≥2 mm, moderate or severe soft tissue involvement, and/or inconstant or constant diplopia.
   Onset of active TED symptoms (as determined by patient records) within 9 months prior to Baseline.
   CAS≥3 or ≥4 (on the 7-item scale) for the most severely affected eye at Screening and Baseline.
   Optionally, clinical diagnosis of Graves' disease associated with active TED.
Exophthalmos ≥3 mm above normal for race and gender or compared with the patient's pre-TED state, in the opinion of the investigator (e.g., according to pre-disease patient photos).
Subjects must be euthyroid with the baseline disease under control, or have mild hypo- or hyperthyroidism (defined as free thyroxine [FT4] and free triiodothyronine [FT3] levels <50% above or below the normal limits) at Screening. Every effort should be made to correct the mild hypo- or hyperthyroidism promptly and to maintain the euthyroid state for the full duration of the clinical trial.
Does not require immediate surgical ophthalmological intervention and is not planning corrective surgery/irradiation during the course of the study.
Diabetic subjects must have HbA1c ≤8.0%.

Exclusion Criteria. Patients nay be ineligible for study participation if they meet any of the following criteria:
Decreased best corrected visual acuity due to optic neuropathy as defined by a decrease in vision of 2 lines on the Snellen chart, new visual field defect, or color defect secondary to optic nerve involvement within the last 6 months.
Corneal decompensation unresponsive to medical management.
Decrease in CAS≥1 in either eye (for chronic/inactive TED) or of ≥2 points in the study eye (for acute/active TED) between Screening and Baseline.
Decrease in proptosis of ≥2 mm in the study eye between Screening and Baseline.
Prior orbital irradiation, orbital decompression, or strabismus surgery.
Intravenous (IV) or oral steroids for the treatment of TED or use of steroid eye drops for the treatment of TED within 6 months prior to Screening.
Corticosteroid use for conditions other than TED within 4 weeks prior to Screening (topical steroids for dermatological conditions and inhaled steroids are allowed).
Previous treatment with rituximab (Rituxan® or MabThera®).
Previous treatment with teprotumumab.
Treatment with tocilizumab (Actemra® or Roactemra®) or any other non-steroid immunosuppressive agent within 6 months prior to Screening.
Use of an investigational agent for any condition within 60 days prior to Screening or anticipated use during the course of the trial.
Identified pre-existing ophthalmic disease that, in the judgment of the Investigator, would preclude study participation or complicate interpretation of study results.
Malignant condition in the past 12 months (except successfully treated basal/squamous cell carcinoma of the skin).
Pregnant or lactating women.
Current drug or alcohol abuse, or history of either within the previous 2 years, in the opinion of the Investigator or as reported by the subject.
Biopsy-proven or clinically suspected inflammatory bowel disease (e.g., diarrhea with or without blood or rectal bleeding associated with abdominal pain or cramping/colic, urgency, tenesmus, or incontinence for more than 4 weeks without a confirmed alternative diagnosis OR endoscopic or radiologic evidence of enteritis/colitis without a confirmed alternative diagnosis).
Known hypersensitivity to any of the components of the Study Drug [or prior hypersensitivity reactions to mAbs]
Any other condition that, in the opinion of the Investigator, would preclude inclusion in the study.
Previous enrollment in this study or participation in a prior clinical trial for the Study Drug.
Human immunodeficiency virus, hepatitis C or hepatitis B infections.
Alanine aminotransferase (ALT) or aspartate aminotransferase (AST) >3 times the upper limit of normal (ULN) or estimated glomerular filtration rate of <30 mL/min/1.73 m2 at Screening.

Study Objectives

The overall objective of the study will be to investigate the efficacy, safety, and tolerability of a monoclonal antibody (mAb) or small molecule inhibitor of the insulin-like growth factor-1 receptor (IGF-1R), in the treatment of subjects with acute or chronic TED.

The primary objective is to evaluate the effect of Study Drug versus placebo or teprotumumab on the mean change from Baseline to End of the Treatment Period (Week 12, Week 24, or Week 48) in proptosis measurement or diplopia (measured by improvement in subjective Gorman scale, Goldmann perimeter, or Cervical range of motion method) in the study eye in subjects with Chronic/inactive TED.

Other objectives include the following:

Evaluate the effect of Study Drug versus placebo or teprotumumab on the mean change from Baseline to Week 12, Week 24, and/or Week 48 in the Graves' Ophthalmopathy Quality of Life (GO-QoL) questionnaire appearance and visual functioning sub-scales.

Evaluate the effect of Study Drug versus placebo or teprotumumab on the proptosis responder rate (i.e., the percentage of subjects with a ≥2 mm reduction from Baseline in the study eye without deterioration [≥2 mm increase] of proptosis in the fellow eye) at Week 12, Week 24, and/or Week 48.

Evaluate the effect of Study Drug versus placebo or teprotumumab on the binocular diplopia responder rate (i.e., the percentage of subjects with baseline diplopia >0 who have a reduction of ≥1 grade) at Week 12, Week 24, and/or Week 48.

Evaluate the effect of Study Drug versus placebo or teprotumumab on the mean change from Baseline to Week 12, Week 24, and/or Week 48 in orbital pain (measured on a visual analog scale [VAS]).

Evaluate the effect of Study Drug versus placebo or teprotumumab on the mean change from Baseline to Week 12, Week 24, and/or Week 48 in the Muscle Diameter Index (MDI) and Pixel Value Ratio (PVR) for the inferior rectus, superior rectus, the medial rectus, lateral rectus and orbital fat (measured by magnetic resonance imaging [MRI]) on subjects where MRI is obtained.

Evaluate the effect of Study Drug versus placebo or teprotumumab on percentage of subjects with a Clinical Activity Score (CAS) of ≥3 in the study eye at Week 12, Week 24, and/or Week 48.

Evaluate the effect of Study Drug versus placebo or teprotumumab on the mean change from Baseline to Week 12, Week 24, and/or Week 48 in the circumference of calf and area of the lesion (maximum length and width of lesion) in subjects with baseline pretibial myxedema (PTM).

Evaluate the effect of Study Drug versus placebo or teprotumumab on changes from Baseline at Weeks 3, 12, 24, and/or 48 in inflammatory and fibrotic biomarkers.

Evaluate the effect of Study Drug versus placebo or teprotumumab on changes from Baseline at Weeks 3, 12, 24, and/or 48 in transcriptomics associated with IGF-1R inhibition.

Pharmacokinetic and Anti-drug Antibody (ADA) Objectives include the following:

Evaluate pharmacokinetics (PK) of Study Drug to estimate exposure.

Evaluate the immunogenicity of Study Drug.

Safety and Tolerability Objectives include the following:

Assess the safety and tolerability of Study Drug versus placebo or teprotumumab based on adverse event (AE) reports, adverse events of special interest (AESI; hyperglycemia, hearing impairment and muscle spasms), concomitant medication use, ophthalmic examinations, vital signs, clinical safety laboratory evaluations, electrocardiograms (ECGs) and immunogenicity.

Restrictions During Study

The trial will comprise three phases: screening (28 days prior to Day 1), treatment or intervention period (Day 1 to Week 12, Week 24, and/or Week 48), and follow-up for, e.g., 6 weeks of more after the nd of the treatment period. Screening involves one to three visits. During the treatment period, patients will be assessed at Day 1/Baseline and every 3 weeks for 12, 24, or 48 weeks. Efficacy may be assessed throughout the Treatment Period, e.g.:
CAS at Day 1/Baseline and weeks 12 and 24 for a study with a 24-week treatment period, or a similar schedule adjusted for a 12 or 48 week treatment period;
proptosis and diplopia at Day 1/Baseline and weeks 3, 6, 12, 18, and 24 for a study with a 24-week treatment period, or a similar schedule adjusted for a 12 or 48 week treatment period;
PTM at Day 1/Baseline and weeks 12 and 24 for a study with a 24-week treatment period, or a similar schedule adjusted for a 12 or 48 week treatment period; and
orbital pain at Day 1/Baseline and weeks 3, 6, 12, 18, and 24 for a study with a 24-week treatment period, or a similar schedule adjusted for a 12 or 48 week treatment period.

Data from the end of the Treatment Period, i.e. week 12, 24, or 48, will be used to assess the primary and secondary end points. A change of 2 points in the 7-component CAS will be considered to be clinically relevant, as will achievement of a CAS of 0 or 1 in acute/active TED patients. Proptosis will be assessed with the use of a Hertel exophthalmometer. A change of 2 mm will be considered to be clinically relevant. Quality of life will be evaluated with the use of the Graves' ophthalmopathy-specific quality-of-life questionnaire (GO-QoL), comprising two subscales assessed separately or in combination; scores on each subscale as well as the score on the overall GO-QoL scale have a range of 0 to 100 points, with a change of 8 points being considered to be clinically relevant. Subjective diplopia will be assessed by improvement in subjective Gorman scale or Goldmann perimeter or Cervical range of motion method.

Institutional review and ethics committees of the participating centers and the investigators will approve the research protocol. Witnessed, written informed consent will be obtained from all patients. Data will be obtained by the investigators and their staff.

### Moieties/Interventions Used in the Trial

Drugs for evaluation in TED in the present study may include any of the biologic or small molecule drugs listed in Examples 1-22. Teprotumumab may be provided according to its marketed formulation. Other study drugs will be provided as given below or as appropriate. The placebo will be appropriate to the given Study Drug, e.g. IV saline or buffer solution or matching placebo tablet/capsule. Patients will receive equivalent types and numbers of administrations of either a Study Drug listed in Table 1, or teprotumumab, or placebo. Dosages are provided below. For example, for subjects assigned to the teprotumumab group, the drug may be administered every 3 weeks starting with an initial dose of 10 mg per kilogram of body weight, followed by 20 mg per kilogram for the remaining infusions. Dosages or frequency of administration can be altered as deemed appropriate by a clinician or study coordinator.

As disclosed herein for the anti-IGF-R1 antibody drugs listed in Table 1, the lower end of the dose range appropriate for use in TED were estimated from the minimum concentration (Cₘᵢₙ) that would achieve an in vitro IC₅₀ as disclosed in the art. The higher end of the dose range appropriate for use in TED was estimated as three-fold of the recommended phase 2 dose (RP2D) if it is not the maximum tolerated dose (MTD), or 2.5-fold of the RP2D if it is the MTD.

As disclosed herein for the anti-IGF-R1 small molecule drugs listed in Table 1, the lower end of the dose range appropriate for use in TED were estimated from the maximum concentration (Cₘₐₓ) that would achieve an in vitro IC₅₀ as disclosed in the art. The higher end of the dose range appropriate for use in TED was estimated as three-fold of the recommended phase 2 dose (RP2D) if it is not the maximum tolerated dose (MTD), or 2.5-fold of the RP2D if it is the MTD.

Dose ranges in Table 1 are given as total dose for a 3-week interval (antibodies) or daily (small molecules) unless otherwise specified.

TABLE 1
List of Study Drugs.

| | | Drug | Oncology | Dose (s): range and/or | Route, |
|---|---|---|---|---|---|
| Ex. | Drug Name(s) | Type | RP2D | examples for TED | T_{1/2} |
| A | TEPEZZA | Antibody | * | 5-20 mg/kg Q3W; e.g. 10 or 20 | IV; 8 |
| | (teprotumumab) | | | mg/kg Q3W | days |
| 1 | Dalotuzumab | antibody | 10 mg/kg | total dose 1-90 mg/kg or 75-6800 | IV;5 days |
| | (MK-0646) | | QW, | mg Q3W; or 0.6-60 mg/kg or 45- | |
| | | | 20 mg/kg | 4500 mg Q2W; or 0.3-30 mg/kg or | |
| | | | Q2W, | 22-2300 mg QW | |
| | | | 30 mg/kg | | |
| | | | Q3W | | |
| 2 | Ganitumab | antibody | 12 mg/kg | total dose 1-60 mg/kg or 75-4500 | IV; 6-9 |
| | (AMG 479) | | Q2W | mg Q3W; 0.6-40 mg/kg or 45-3000 | days |
| | | | | mg Q2W; 0.3-20 mg/kg; or 22-1500 QW | |
| 3 | Xentuzumab (BI | antibody | 1000 | 1-112 mg/kg or 75-8400 mg Q3W; | IV |
| | 836845) | | mg/kg | or 0.6-75 mg/kg or 45-5700 mg | |
| | | | | Q2W; or 0.3-38 mg/kg or 22-2900 mg QW | |
| 4 | AVE1642 | antibody | 6 mg/kg | total dose 1-60 mg/kg or 75-4500 | IV , 8 |
| | | | Q3W | mg Q3W; or 0.6-40 mg/kg or 45- | days |
| | | | | 3000 mg Q2W; or 0.3-20 mg/kg or | |
| | | | | 22-1500 mg QW | |
| 5 | Figitumumab | antibody | 20 mg/kg | total dose 1-60 mg/kg or 75-4500 | IV; 20 |
| | (CP-751) | | Q3W | mg Q3W; or 0.6-40 mg/kg or 45- | days |
| | | | | 3000 mg Q2W; or 0.3-20 mg/kg or | |
| | | | | 22-1500 mg QW | |
| 6 | Dusigitumab | antibody | 30-45 | total dose 1-75 mg/kg or 75-5700 | IV |
| | (MEDI-573) | | mg/kg | mg Q3W; or 0.6-50 mg/kg or 45- | |
| | | | | 3800 mg Q2W; or 0.3-25 mg/kg or | |
| | | | | 22-1900 mg QW | |
| 7 | Cixutumumab | antibody | 10 mg/kg | total dose 1-45 mg/kg or 75-3400 | IV; 5 |
| | (IMC-A12) | | Q2W | mg Q3W; or 0.6-30 mg + D2/kg or | days |
| | | | | 45-2300 mg Q2W; or 0.3-15 mg/kg | |
| | | | | or 22-1200 mg QW | |
| 8 | BIIB022 | antibody | 30 mg/kg | total dose 1-75 mg/kg or 75-5700 | IV;15 |
| | | | Q3W | mg Q3W; or 0.6-50 mg/kg; or 45- | days |
| | | | | 3800 mg Q2W; or 0.3-25 mg/kg or | |
| | | | | 22-1900 mg | |
| 9 | Robatumumab | antibody | 10 mg/kg | total dose 1-75 mg/kg or 75-5700 | IV |
| | (SCH 717454) | | Q2W | mg Q3W; or 0.6-50 mg/kg or 45- | |
| | | | | 3800 mg Q2W; or 0.3-25 mg/kg or | |
| | | | | 22-1900 mg QW | |
| 10 | Linsitinib (OSI-906) | Small | 150 mg | QD: 10-750 mg/day for continuous | oral; 2-4 |
| | | molecule | BID | dosing; or 10-1500 mg/day for | hrs |
| | | | (tested in | intermittent dosing (for up to 7 days | |
| | | | Ph2); or | of every 14 days); BID: 6-500 mg | |
| | | | 600 mg | for continuous dosing; 6-1000 mg | |
| | | | QD for | for intermittent dosing (for up to 7 | |
| | | | days 1-3 | days of every 14 days); TID: 3-250 | |
| | | | or 1-7 for | mg for continuous dosing; 3-500 | |
| | | | every 14 | mg for intermittent dosing (for up | |
| | | | days | to 7 days of every 14 days) | |
| 11 | Picropodophyllin | small | 390 mg | QD: 20-2000 mg; BID: 13-1400 | oral, e.g. |
| | (AXL1717) | molecule | BID | mg; or TID: 6-700 mg | susp. |
| | | | (tested in | | |
| | | | Ph2 | | |
| | | | NSCLC) | | |
| 12 | GTx-134 | small | ** | 1-2000 mg QD; or 0.6-1400 mg | oral |
| | | molecule | | BID; or 0.3-700 mg TID. | |
| 13 | AG1024 | small | ** | 1-2000 mg QD; or 0.6-1400 mg | oral |
| | | molecule | | BID; or 0.3-700 mg TID. | |
| 14 | BMS-536924 | small | ** | 1-2000 mg QD; or 0.6-1400 mg | oral |
| | | molecule | | BID; or 0.3-700 mg TID. | |
| 15 | NVP-AEW541 | small | ** | 1-2000 mg QD; or 0.6-1400 mg | oral |
| | | molecule | | BID; or 0.3-700 mg TID. | |
| 16 | BMS-754807 | small | | 5-600 mg QD; 3-400 mg BID; 1- | oral |
| | | molecule | | 200 mg TID | |
| 17 | GSK1838705A | small | ** | 1-2000 mg QD; or 0.6-1400 mg | oral |
| | | molecule | | BID; or 0.3-700 mg TID. | |
| 18 | BMS-554417 | small | ** | 1-2000 mg QD; or 0.6-1400 mg | oral |
| | | molecule | | BID; or 0.3-700 mg TID. | |
| 19 | NVP-ADW742 | small | ** | 1-2000 mg QD; or 0.6-1400 mg | oral |
| | | molecule | | BID; or 0.3-700 mg TID. | |
| 20 | GSK1904529A | small | ** | 1-2000 mg QD; or 0.6-1400 mg | oral |
| | | molecule | | BID; or 0.3-700 mg TID. | |
| 21 | KW-2450 | small | ** | 1-100 mg QD; 0.7-70 mg BID; 0.3- | oral |
| | | molecule | | 30 mg TID | |
| 22 | PL-2258 / PL-225B | small | | 1-2000 mg QD; or 0.6-1400 mg | Oral; 21d |
| | | molecule | | BID; or 0.3-700 mg TID. | |
| 23 | INSM-18, | small | ** | 1-2000 mg QD; or 0.6-1400 mg | oral |
| | nordihydroguaiaretic | molecule | | BID; or 0.3-700 mg TID. | |
| | acid (NDGA)/ | | | | |
| | Masoprocol, | | | | |
| | Actinex, TT-100 | | | | |
| 24 | AZD3463 | small | ** | 1-2000 mg QD; or 0.6-1400 mg | oral |
| | | molecule | | BID; or 0.3-700 mg TID. | |
| 25 | AZD9362 | small | | 1-2000 mg QD; or 0.6-1400 mg | oral |
| | | molecule | | BID; or 0.3-700 mg TID. | |
| 26 | BI885578 | small | ** | 1-2000 mg QD; or 0.6-1400 mg | oral |
| | | molecule | | BID; or 0.3-700 mg TID. | |
| 27 | BI893923 | small | ** | 1-2000 mg QD; or 0.6-1400 mg | oral |
| | | molecule | | BID; or 0.3-700 mg TID. | |
| 28 | XL-228 | small | ** | 1-2000 mg QD; or 0.6-1400 mg | oral |
| | | molecule | | BID; or 0.3-700 mg TID. | |
| 29 | A-928605 | small | ** | 1-2000 mg QD; or 0.6-1400 mg | oral |
| | | molecule | | BID; or 0.3-700 mg TID. | |
| 30 | Istiratumab (MM-141) | antibody | 2.8 g IV | 1-112 mg/kg or 75-8400 mg Q3W; | IV |
| | | | q2w | or 0.7-75 mg/kg or 45-5700 mg | |
| | | | | Q2W; or 0.3-38 mg/kg or 22-2900 mg QW | |

In Table 1 above, * indicates that RP2D is known in the art; and ** indicates that prior clinical experience with the IGF-1R inhibitor is not known to be published.

In some embodiments, other IGF-1R antibodies may be useful as described herein and are encompassed within the present disclosure. In some embodiments, if prior clinical experience with an anti-IGF-R1 antibody is not published, a dosage appropriate for use for the present disclosure may be 1-112 mg/kg or 75-8400 mg every 3 weeks (Q3W); or 0.6-75 mg/kg or 45-5700 mg every 2 weeks (Q2W); or 0.3-38 mg/kg or 22-2900 mg weekly (QW). In some embodiments, the doses are also useful when given Q4W.

In some embodiments, additional dosages of antibodies disclosed herein appropriate for use include:
1-30 mg/kg or 75-2250 mg (e.g., Q3W SC);
0.6-20 mg/kg or 1500 mg (e.g., Q2W SC);
0.3-10 mg/kg or 750 mg (e.g., QW SC);
   and/or
1-20 mg/kg or 75-1500 mg (e.g., Q3W SC);
0.6-13.5 mg/kg or 1000 mg (e.g., Q2W SC);
0.3-7 mg/kg or 500 mg (e.g., QW SC).
   In some embodiments, the above dosages may also be appropriate when dosed a 1, 2, 3, or 4 week intervals, and/or when dosed IV or SC.

In some embodiments, other IGF-1R small molecule drugs may be useful as described herein and are encompassed within the present disclosure. In some embodiments, if prior clinical experience with a small molecule IGF-1R inhibitor drug is not published, a dosage appropriate for use for the present disclosure may be 1-2000 mg for once daily administration (QD); or 0.6-1400 mg for twice daily administration (BID); or 0.3-700 mg for three-times daily administration (TID).

On Day 1 of the Treatment Period, subjects will be randomized in an appropriate (e.g., a 2:1 or 1:1 ratio, optionally stratified by duration of disease) to the Study Drug. Placebo doses (IV saline or buffer solution or matching placebo tablet/capsule) will be used to maintain the blind due to differing administration schedules and/or methods of administration with the active comparator.

### Detailed Study Procedures

At the Baseline (Day 1) Visit, the eye with the more significant proptosis may be defined as the “study eye.” If both eyes are affected equally, the Investigator may choose the “study eye.” Both eyes will be assessed for efficacy but the study eye may be used to assess the primary outcome measure.

Efficacy will be assessed by proptosis (measured as exophthalmos evaluation of the Clinical Measures of Severity using a Hertel instrument provided by the Sponsor for consistency in measurement), quality of life (using GO-QoL questionnaire), diplopia (measured as part of the Clinical Measures of Severity or using Goldmann perimeter or cervical range of motion method), CAS (7-item or 10-item scale), orbital pain (using a 10-cm VAS), orbital MRI, and/or PTM (calf circumference and area of lesion).

Blood samples for Study Drug PK assessment will be collected prior to dosing on Day 1 and at End of Treatment Period, e.g., Week 12, Week 24, or Week 48. Blood samples may be collected and analyzed for inflammatory and fibrotic biomarkers and evaluated for transcriptomics associated with IGF-1R inhibition prior to treatment on Day 1 and after treatment throughout the study, e.g. for a 24-week treatment period, at Weeks 3, 12, and 24.

Safety will be assessed via AE and concomitant medication use monitoring, immunogenicity testing, ophthalmic examinations, vital signs, clinical safety laboratory evaluations (complete blood count and chemistry (including thyroid panel and HbA1c), pregnancy testing (if applicable), and ECGs.

A summary of the study procedures, including the timing for each, is provided in the Schedule of Assessments (Table 2).

Informed Consent: Informed consent will be obtained from each subject during Screening.

Inclusion/Exclusion Criteria: Inclusion/exclusion criteria will be reviewed with each subject at Screening and on the Day 1/Baseline visit.

Demographics: Demographic data may be obtained from each subject during Screening.

Medical History: Medical history, including thyroid disease history and treatment, TED history and treatment, and tobacco use history, will be obtained from each subject at Screening and on the Day 1/Baseline visit. TED must be either i) acute/active TED (onset of symptoms within 9 months prior to Baseline) or ii) stable, chronic/inactive (not progressing, non-sight threatening but appreciable impact on daily life) with TED diagnosed >2 years, but no longer than 7 years prior to Screening.

Weight: Weight may be recorded at Screening, and throughout the study, e.g. for a 24-week treatment period, Week 12/Month 3, and Week 24/Month 6. Dosing may be adjusted if there is a change in weight during the Treatment Period. The weight obtained mid-study can be used in dose calculations for later doses.

Randomization: On Day 1 (Baseline), subjects will be randomized and receive the first dose of study drug. Baseline assessments will be performed prior to dosing.

Subjects will be randomized as described herein to receive: (a) Study Drug; or (b) placebo or (c) teprotumumab—i.e., the study may be designed with two arms to compare Study Drug to either placebo or teprotumumab, or may be designed with three arms to compare Study Drug, placebo, and teprotumumab. Study Drug will be given as described herein. Teprotumumab Infusion: Infusions will take place on Day 1 (Baseline), and per marketed dosing thereafter. Placebo administration will match that of Study Drug or teprotumumab as appropriate.

Phone (email) contact for safety—day after infusion: Phone (or email) contact by research staff focusing on safety and tolerability aspects will be made the day after infusion for the first and second infusions (Day 1/Baseline and Day 3), and thereafter as deemed appropriate. In addition, subjects who experience an infusion-associated event after any subsequent infusion will also be contacted by phone (or email) by research staff the day after the infusion, and thereafter as deemed appropriate.

### Efficacy Assessments

Clinical Activity Score (CAS): CAS will be obtained from each subject at Screening, Day 1/Baseline, and throughout the study, e.g. for a 24-week treatment period, Week 12/Month 3, and Week 24/Month 6. For patients entering a chronic/inactive study, CAS must be ≤1 in both eyes at the Screening and Baseline visits.

Clinical Measures of Severity—includes proptosis and diplopia: Clinical measures of severity will be obtained at Screening; at Day 1/Baseline, and throughout the study, e.g. for a 24-week treatment period, Week 3, Week 6, Week 12/Month 3, Week 18, and Week 24/Month 6 of the Treatment Period; and Week 30 of the Follow-up Period.

Subjects who have a ≥2 mm decrease in proptosis in the study eye from Screening are not eligible for randomization.

Pretibial myxedema (PTM) assessment: PTM assessment may optionally be performed at Day 1/Baseline and throughout the study, e.g. for a 24-week treatment period, Week 12/Month 3, and Week 24/Month 6.

Orbital pain by 10 cm visual analog scale: Orbital pain may be assessed on Day 1/Baseline and throughout the study, e.g. for a 24-week treatment period, Week 3, Week 6, Week 12/Month 3, Week 18, and Week 24/Month 6.

### Safety Assessments

Pregnancy test: Pregnancy tests will be administered at all visits. Serum pregnancy test at Screening and Week 48 (or 6 months after last infusion if withdrawn early from treatment). Urine pregnancy tests prior to dosing at all other visits, as applicable. Perform for female subjects of childbearing potential (including those with an onset of menopause <2 years prior to Screening, non-therapy-induced amenorrhea for <12 months prior to Screening, or not surgically sterile [absence of ovaries and/or uterus]).

Ophthalmic exam: Ophthalmic exam will be performed at Screening, Day 1/Baseline, and throughout the study, e.g. for a 24-week treatment period, Week 6, Week 12/Month 3, Week 18, and Week 24/Month 6.

Best corrected visual acuity, pupil exam, color vision assessment, Ishihara color plates (or equivalent) or related red desaturation, intraocular pressure, and slit lamp exam. If significant abnormalities are noted compared to previous visits, including a loss of 2 lines or more of vision, development of pupil abnormalities including afferent pupillary defect, rise in intraocular pressure, development of corneal infiltrates or other abnormalities not here specified but of concern to the ophthalmologist, further investigations of visual function will be conducted according to the ophthalmologist decision.

Subjects who have decreased best-corrected visual acuity due to optic neuropathy (defined by a decrease in vision of 2 lines on the Snellen chart, new visual field defect, or color defect secondary to optic nerve involvement within the last 6 months) are not eligible for randomization.

Vital signs: Vital signs (blood pressure, heart rate, respiratory rate, temperature) will be measured at all clinic visits. Vital signs will be measured pre- and post-dose on Day 1, and pre-dose on other dose/infusion days. Additional vital signs will be monitored if infusion-associated AEs occur.

12-Lead ECG: Electrocardiogram (ECG) will be performed at Screening, Day 1/Baseline, and throughout the study, e.g. for a 24-week treatment period, Week 3, Week 6, Week 12/Month 3, and Week 24/Month 6.

### Clinical Laboratory Tests

Chemistry: Chemistry may be assessed at Screening, Day 1/Baseline, and throughout the study, e.g. for a 24-week treatment period, Week 3, Week 6, Week 12/Month 3, Week 18, Week 24/Month 6, Week 30, and Week 36.

Thyroid (FT3, FT4, THS): Thyroid levels may be assessed at Screening, Day 1/Baseline, and throughout the study, e.g. for a 24-week treatment period, Week 3, Week 6, Week 12/Month 3, Week 18, Week 24/Month 6, Week 30, and Week 36. Subjects must be euthyroid with the baseline disease under control or have mild hypo- or hyperthyroidism (defined as FT4 and FT3 levels <50% above or below the normal limits). Every effort should be made to correct the mild hypo- or hyperthyroidism promptly and to maintain the euthyroid state for the full duration of the clinical trial.

Hematology: Hematology may be assessed at Screening, Day 1/Baseline, and throughout the study, e.g. for a 24-week treatment period, Week 3, Week 6, Week 12/Month 3, Week 18, Week 24/Month 6, Week 30, and Week 36.

HbA1c: HbA1c levels may be assessed at Screening, and throughout the study, e.g. for a 24-week treatment period, Week 12/Month 3, and Week 24/Month 6. HbA1c must be ≤8.0% for randomization. If the HbA1c is elevated and considered clinically significant at any time point after Screening, it will be repeated approximately every 90 days until it returns to normal or baseline value.

ADA/Nab samples: Anti-drug antibody (ADA)/neutralizing antibody (Nab) levels may be obtained on Day 1/Baseline, and throughout the study, e.g. for a 24-week treatment period, Week 3, Week 12/Month 3, and Week 24/Month 6. If a sample is positive in the ADA test, after confirmatory and reactive titer testing, the sample will then be tested for NAb. If the subject tests positive for NAb, he/she may be followed until levels either revert to Baseline or the subject's value decreases or remains stable. Any subject with a positive NAb test at the end of the Treatment Period (or PW) may continue to be followed until the subject's value decreases or remains stable.

AE/SAE Assessment: AEs/SAEs will be assessed periodically, up to and including at every visit. AEs that occur within 2 weeks prior to Day 1 and prior to dosing on Day 1 will be considered baseline signs/symptoms. AEs occurring or worsening after the dose on Day 1 through the end of the Treatment Period will be considered treatment-emergent AEs (TEAEs). AEs occurring or worsening during the Follow-Up Period will be considered post-dose AEs. All SAEs that occur from the signing of informed consent through 30 days after study discontinuation will be recorded.

Concomitant medications: Concomitant medications will be assessed periodically, up to and including at every visit.

Graves' Ophthalmopathy Quality of Life (GO-QoL) Questionnaire: GO-QoL may be assessed at Day 1/Baseline, and periodically throughout the study, e.g. for a 24-week treatment period, Week 6, Week 12/Month 3, and Week 24/Month 6.

PK Samples: PK samples may be collected prior to, and at the end of, the dosing or infusion on Day 1, and periodically throughout the study, e.g. for a 24-week treatment period, Week 3 and Week 12/Month 3 of the Treatment Period, and a single sample may be collected at the end of the Treatment Period. PK samples will not be collected for subjects who prematurely discontinue from the Treatment Period.

Biomarker Samples: Biomarker samples may be collected on Day 1 and throughout the study, e.g. for a 24-week treatment period, Week 3 and Week 12/Month 3 of the Treatment Period, and a single sample may be collected at the end of the Treatment Period.

Magnetic Resonance Imaging (MRI): Subjects may undergo MRI on Day 1 and at the visit at the end of the Treatment Period.

### Randomization and Masking the Trial

The randomized trial is designed to assess efficacy and safety. For a study of chronic/inactive TED, patients will be randomly assigned in the (optionally) double-masked Treatment Period to one of three treatment groups in, e.g., a 1:1, 2:1, or 3:1 ratio in blocks of two, stratified by duration of chronic/inactive disease, ≤2 years or >2 years.

Study pharmacists who are aware of the trial-group assignments may prepare masked doses and/or infusions if needed. The on-site principal investigators will identify a patient's intervention or Treatment Group (Study Drug, active control, or placebo) only in the case of an emergency.

### Calculation of Clinical Activity Score (CAS)

The clinical activity score consists of seven components: spontaneous retrobulbar pain, pain on attempted eye movements (upward, side-to-side, and downward gazes), conjunctival redness, redness of the eyelids, chemosis, swelling of the caruncle/plica, and swelling of the eyelids. Each component will be scored as present or absent, 1 or 0. The score at each efficacy assessment will be the sum of all items present to give a range of 0-7, where 0 or 1 constitutes inactive disease and 7 constitutes severe active ophthalmopathy. A change of ≥2 points will be considered clinically meaningful, as would achievement of a CAS score of 0 or 1 in patients with acute/active disease.

### Evaluation of Graves' Ophthalmopathy Quality of Life (GO-QoL)

Quality of life will be evaluated with the use of the GO quality of life questionnaire. The questionnaire has two self-assessment subscales; one covering impact of visual function on daily activities, the other assesses the impact of self-perceived appearance. The visual function subscale covers activities such as driving, walking outdoors, reading, watching television, etc. The appearance subscale asks the subject questions such as whether ophthalmopathy has altered the subject's appearance, caused other people to have a negative reaction to the subject, caused social isolation, and caused the subject to try to mask his or her appearance. Each subscale has 8 questions which are answered with: yes—very much so; yes—a little; or no—not at all. Each question is scored 0-2, respectively, and the total raw score is then mathematically transformed to a 0-100 scale, where 0 represents the most negative impact on quality of life, and 100 represents no impact. A change of ≥8 points on the 0-100 scale has been shown to be clinically meaningful. The combined score takes raw scores from both subscales and again transforms them to a single 0-100 scale.

### Assessment of Gorman Grading of Diplopia

The Gorman assessment of subjective diplopia includes four categories: no diplopia (absent), diplopia when the patient is tired or awakening (intermittent), diplopia at extremes of gaze (inconstant), and continuous diplopia in the primary or reading position (constant). Patients are scored according to which grade of diplopia they are experiencing. An improvement of ≥1 grade is considered clinically meaningful.

### Electrocardiogram

A 12-lead ECG may be performed as described in the Schedule of Events (Table 2) for all subjects or at the discretion of the Investigator. When a subject experiences an AE suspected to be an IR, a 12-lead ECG may also be performed.

Single 12-lead ECG recordings may be made at Screening, Baseline (Day 1), and periodically throughout the study, e.g. for a 24-week treatment period, Week 3, Week 6, Week 12/Month 3, and Week 24/Month 6, after the subject has been in the supine position for at least 5 minutes. A single repeat measurement is permitted at Screening for eligibility determination. Measurements of the following intervals may be recorded and reported: RR interval, PR interval, QRS width, QT interval, and QTcF. Assessments should include comments on clinical significance, whether the tracings are normal or abnormal; rhythm; presence of arrhythmia or conduction defects; morphology; any evidence of myocardial infarction; or ST-segment, T-Wave, and U-Wave abnormalities.

### Clinical Laboratory Safety Tests

Blood (for hematology, clinical chemistry, thyroid measurements) may be collected at Screening; at Day 1, and periodically throughout the study, e.g. for a 24-week treatment period, Week 3, Week 6, Week 12/Month 3, Week 18, and Week 24/Month 6 of the Treatment Period, and Week 30 and Week 36 of the Follow-up Period.

HbA1c may be measured at Screening and periodically throughout the study, e.g. for a 24-week treatment period, Week 12/Month 3 of the Treatment Period, and Week 24/Month 6 of the Follow-up Period. HbA1c must be ≤8.0% for randomization. If the HbA1c is elevated and considered clinically significant at any time point after Screening, it will be repeated approximately every 90 days until it returns to normal or baseline value.

Anti-drug antibodies (ADA)/neutralizing antibodies (Nab) may be measured at Day 1, and periodically throughout the study, e.g. for a 24-week treatment period, Week 3, Week 12/Month 3, and Week 24/Month 6 of the Treatment Period. If a sample is positive in the ADA test, after confirmatory and reactive titer testing, the sample will then be tested for NAb. If the subject tests positive for NAb, he/she may be followed until levels either revert to Baseline or the subject's value decreases or remains stable. Any subject with a positive NAb test at the end of the Treatment Period (or PW) may continue to be followed until the subject's value decreases or remains stable.

Safety laboratory assessments may include:

Pregnancy Test: Serum pregnancy test at Screening and Week 48 (or 6 months after last dose or infusion). Urine pregnancy tests prior to dosing at all other visits, as applicable. Perform for female subjects of childbearing potential (including those with an onset of menopause ≤2 years prior to Screening, non-therapy-induced amenorrhea for <12 months prior to Screening, or not surgically sterile [absence of ovaries and/or uterus]).

Ophthalmic exam: best corrected visual acuity, pupil exam, color vision assessment, Ishihara color plates (or equivalent) or related red desaturation, intraocular pressure, and slit lamp exam. If significant abnormalities are noted compared to previous visits, including a loss of 2 lines or more of vision, development of pupil abnormalities including afferent pupillary defect, rise in intraocular pressure, development of corneal infiltrates or other abnormalities not here specified but of concern to the ophthalmologist, further investigations of visual function will be conducted according to the ophthalmologist decision

Vital Signs: blood pressure, heart rate, respiratory rate, and temperature will be measured at all clinic visits. Vital signs will be measured pre- and post-infusion on Day 1 and Week 3, and pre-dose on all other infusion days. Additional vital signs will be monitored if infusion-associated AEs occur.

### Outcomes of Trial

Patients who have a response may be defined as those who meet the primary end point at week 24. This end point may comprise a reduction of 2 mm or more in proptosis in the study eye in the absence of a corresponding amount of worsening in the non-study eye, or average or median change from baseline in proptosis, or a reduction in diplopia of ≥1 grade in subjects with baseline diplopia >0. Secondary end points may include proptosis, diplopia, and the CAS (both measured as continuous variables over time), orbital pain, MDI and PVR for the inferior rectus, superior rectus, the medial rectus, lateral rectus and orbital fat, circumference of calf and area of the lesion in subjects with baseline PTM, inflammatory and fibrotic biomarkers, transcriptomics associated with IGF-1R inhibition, and assessment of the patient's quality of life with the use of the GO-QOL instrument (which includes two subscales that measure limitations in visual functioning and psychosocial functioning as a consequence of changed physical appearance). Patients may also be categorized according to their level of response. Safety will be assessed according to the incidence of adverse events, serious adverse events, and withdrawals due to adverse events.

### Results

It is expected that IGF-1R inhibitors described herein will, when tested as Study Drugs in a clinical study as disclosed herein for either acute/active or chronic/inactive TED, have efficacy in the outcome measures of TED described herein or as modified by one of skill in the art.

Table 2 below sets forth an example of a schedule of assessments assuming a 24-week Treatment Period and either using teprotumumab as an active control or mimicking it, i.e., dosing on a 3-week cycle by infusion. This table is presented as an example for illustrative purposes, and is not meant to be inconsistent with the guidance above. Those of skill in the art will understand how to modify such a schedule in the event of a different dosing schedule or route of administration for example as might be expected with, e.g., a study of a subcutaneously-delivered product administered via autoinjector on a QW or Q2W schedule, or a study of an orally bioavailable small molecule drug dosed QD and compared to placebo as opposed to active teprotumumab control.

TABLE 2
Schedule of Assessments

| | | Treatment Period ² | | | | | | | | | | | | Follow- |
|---|---|---|---|---|---|---|---|---|---|---|---|---|---|---|
| Study Visit | | | | | | | | | | 9/ | Follow-Up Period ³ | | | Up |
| Week (W)/ | Screening ¹ | 1 | | | | 5 | | | | PW1 ⁴ | 10/ | 11 ⁶ | | Contact |
| Month (M) | S1/S2/S3 | Day | 2 | 3 | 4 | W12/ | 6 | 7 | 8 | W24/ | PW2 ⁵ | W36 | W36 | 12 ⁷ |
| Visit Window | −28 | 18 | W3 | W6 | W9 | M3 | W15 | W18 | W21 | M6 | W30 | visit | contact | W48 |
| (±days) | days | (±3) | (±3) | (±3) | (±3) | (±3) | (±3) | (±3) | (±3) | (±7) | (±7) | (±7) | (±7) | (±7) |
| Informed | X | | | | | | | | | | | | | |
| consent | | | | | | | | | | | | | | |
| Review inc/exc | X | X | | | | | | | | | | | | |
| criteria | | | | | | | | | | | | | | |
| Demographics | X | | | | | | | | | | | | | |
| Medical | X ¹⁰ | X | | | | | | | | | | | | |
| history ⁹ | | | | | | | | | | | | | | |
| Weight ¹¹ | X | | | | | X | | | | X | | | | |
| Randomization ¹² | | X ⁸ | | | | | | | | | | | | |
| Teprotumumab, | | X | X | X | X | X | X | X | X | | | | | |
| study drug, or | | | | | | | | | | | | | | |
| placebo | | | | | | | | | | | | | | |
| infusion | | | | | | | | | | | | | | |
| Phone (email) | | X | X | | | | | | | | | | | |
| contact for | | | | | | | | | | | | | | |
| safety-day | | | | | | | | | | | | | | |
| after infusion ¹³ | | | | | | | | | | | | | | |
| Efficacy | | | | | | | | | | | | | | |
| assessments | | | | | | | | | | | | | | |
| CAS ¹⁴ | X | X | | | | X | | | | X | | | | |
| Clinical | X | X ¹⁵ | X | X | | X | | X | | X | X | | | |
| Measures of | | | | | | | | | | | | | | |
| Severity- | | | | | | | | | | | | | | |
| includes | | | | | | | | | | | | | | |
| proptosis and | | | | | | | | | | | | | | |
| diplopia | | | | | | | | | | | | | | |
| PTM | | X | | | | X | | | | X | | | | |
| assessment ¹⁶ | | | | | | | | | | | | | | |
| Orbital pain | | X | X | X | | X | | X | | X | | | | |
| by 10 cm | | | | | | | | | | | | | | |
| visual analog | | | | | | | | | | | | | | |
| scale | | | | | | | | | | | | | | |
| Safety | | | | | | | | | | | | | | |
| assessments | | | | | | | | | | | | | | |
| Pregnancy | X | X | X | X | X | X | X | X | X | X | X | X | | X |
| test ¹⁷ | | | | | | | | | | | | | | |
| Ophthalmic | X ¹⁹ | X | | | | | | | | X | | | | |
| exam ¹⁸ | | | | | | | | | | | | | | |
| Vital signs ²⁰ | X | X ²⁰ | X ²⁰ | X | X | X | X | X | X | X | X | X | | |
| 12-Lead | X | X | X | X | | X | | | | X | | | | |
| ECG | | | | | | | | | | | | | | |
| Clinical | | | | | | | | | | | | | | |
| laboratory | | | | | | | | | | | | | | |
| tests | | | | | | | | | | | | | | |
| Chemistry | X | X | X | X | | X | | X | | X | X | X | | |
| Thyroid | | X | | | | | | X | | X | X | X | | |
| (FT3, FT4, | | | | | | | | | | | | | | |
| TSH) ²¹ | | | | | | | | | | | | | | |
| Hematology | | X | X | X | | | | X | | X | X | X | | |
| HbA1c ²² | | | | | | | | | | X | | | | |
| ADA/NAb | | X | X | | | X | | | | X ²⁴ | | | | |
| samples ²³ | | | | | | | | | | | | | | |
| AE, SAE | X | X | X | X | X | X | X | X | X | X | X | X | X | |
| assessment ²⁵ | | | | | | | | | | | | | | |
| Concomitant | X | X | X | X | X | X | X | X | X | X | X | X | | |
| medications | | | | | | | | | | | | | | |
| GO-QoL | | X | | X | | X | | | | X | | | | |
| Questionnaire | | | | | | | | | | | | | | |
| PK samples ²⁶ | | X | X | | | X | | | | X ²⁴ | | | | |
| Biomarker | | X | X | | | X | | | | X | | | | |
| samples | | | | | | | | | | | | | | |
| MRI ²⁷ | | X | | | | | | | | X | | | | |

ADA = anti-drug antibody;
AE = adverse event;
AESI = adverse event of special interest;
CAS = Clinical Activity Score;
ECG = electrocardiogram;
FT3 = free triiodothyronine;
FT4 = free thyroxine;
FU = Follow-Up;
GO-QoL = Graves' Ophthalmopathy Quality of Life Questionnaire;
HbA1c = glycated hemoglobin;
M = month;
MRI = magnetic resonance imaging;
NAb = neutralizing antibody;
PK = pharmacokinetic;
PTM = pretibial myxedema;
PW = premature withdrawal;
q3W = once every 3 weeks;
SAE = serious adverse event;
TEAE = treatment-emergent adverse event;
TED = thyroid eye disease;
TSH = thyroid stimulating hormone;
W = week.
Footnotes:
¹ Screening procedures can take place over more than 1 day/clinic visit provided consent is obtained first and all assessments are completed within the designated window.
² Double-masked Treatment Period. Subjects who are proptosis non-responders at Week 24 are eligible to enroll in an open-label extension study in which all subjects will receive Study Drug (10 mg/kg for the first infusion and 20 mg/kg for the remaining 7 infusions).
³ Proptosis responders and non-responders who choose not to enroll in the open-label extension study will participate in a 12-week Follow-Up Period.
⁴ If a subject prematurely discontinues study drug during the Treatment Period, they will return for a clinic visit and undergo the Week 24 assessments, with the exception of the collection of blood samples for PK and ADA evaluations. Subjects will be encouraged to continue study participation in the Follow-Up Period.
⁵ If a subject prematurely discontinues from the study between Week 24 and Week 30 of the Follow-Up Period, they will return for a clinic visit and undergo the Week 30 assessments prior to discharge.
⁶ All subjects will be contacted via phone or email at Week 36, except subjects who have an ongoing SAE or AESI at the Week 30 visit. Subjects who have an ongoing SAE or AESI at the Week 30 visit will return to the clinic at Week 36.
⁷ Women of childbearing potential will be contacted via phone or email at Week 48 to inquire if they have missed a menstrual cycle and will return to the clinic for a serum pregnancy test if required.
⁸ On Day 1 (Baseline), subjects will be randomized and receive the first dose of study drug; however, Baseline assessments will be performed prior to dosing.
⁹ Medical history including thyroid disease history and treatment, TED history and treatment and tobacco use history.
¹⁰ TED must be stable, chronic/inactive (not progressing, non-sight threatening but appreciable impact on daily life) with TED diagnosed > 2 years, but no longer than 7 years prior to Screening.
¹¹ Dosing will be adjusted if there is a change in weight during the Treatment Period. The weight obtained at Week 12 can be used in dose calculations beginning at Week 12 or Week 15.
¹² Subjects will be randomized in a 1:1 ratio (stratified by duration of chronic/inactive disease) to receive either: a) Study Drug (10 mg/kg on Day 1 followed by 20 mg/kg q3W for the remaining 7 infusions) or b) placebo or teprotumumab (q3W for all 8 infusions).
¹³ Phone (or email) contact by research staff focusing on safety and tolerability aspects will be made the day after infusion for the first and second infusions, and thereafter as deemed appropriate. In addition, subjects who experience an infusion-associated event after any subsequent infusion will also be contacted by phone (or email) by research staff the day after the infusion, and thereafter as deemed appropriate.
¹⁴ CAS must be ≤ 1 in both eyes at the Screening and Baseline visits.
¹⁵ Subjects who have a ≥ 2 mm decrease in proptosis in the study eye from Screening are not eligible for randomization.
¹⁶ Assess for presence or absence of PTM on Day 1, Week 12, and Week 24. If present, measurements of calf and lesion will be taken.
¹⁷ Serum pregnancy test at Screening and Week 48 (or 6 months after last infusion if withdrawn early from treatment). Urine pregnancy tests prior to dosing at all other visits, as applicable. Perform for female subjects of childbearing potential (including those with an onset of menopause < 2 years prior to Screening, non-therapy-induced amenorrhea for < 12 months prior to Screening, or not surgically sterile [absence of ovaries and/or uterus]).
¹⁸ Ophthalmic exam: best corrected visual acuity, pupil exam, color vision assessment, Ishihara color plates (or equivalent) or related red desaturation, intraocular pressure, and slit lamp exam. If significant abnormalities are noted compared to previous visits, including a loss of 2 lines or more of vision, development of pupil abnormalities including afferent pupillary defect, rise in intraocular pressure, development of corneal infiltrates or other abnormalities not here specified but of concern to the ophthalmologist, further investigations of visual function will be conducted according to the ophthalmologist decision.
¹⁹ Subjects who have decreased best-corrected visual acuity due to optic neuropathy (defined by a decrease in vision of 2 lines on the Snellen chart, new visual field defect, or color defect secondary to optic nerve involvement within the last 6 months) are not eligible for randomization.
²⁰ Vital signs (blood pressure, heart rate, respiratory rate, temperature) will be measured at all clinic visits. Vital signs will be measured pre- and post-infusion on Day 1 and Week 3, and pre-dose on all other infusion days. Additional vital signs will be monitored if infusion-associated AEs occur.
²¹ Subjects must be euthyroid with the baseline disease under control or have mild hypo- or hyperthyroidism (defined as FT4 and FT3 levels < 50% above or below the normal limits). Every effort should be made to correct the mild hypo- or hyperthyroidism promptly and to maintain the euthyroid state for the full duration of the clinical trial.
²² HbA1c must be ≤ 8.0% for randomization. If the HbA1c is elevated and considered clinically significant at any time point after Screening, it will be repeated approximately every 90 days until it returns to normal or baseline value.
23 If a sample is positive in the ADA test, after confirmatory and reactive titer testing, the sample will then be tested for NAb. If the subject tests positive for NAb, he/she may be followed until levels either revert to Baseline or the subject's value decreases or remains stable. Any subject with a positive NAb test at Week 24 (or PW) may continue to be followed until the subject's value decreases or remains stable.
²⁴ Not collected for subjects who prematurely discontinue from the Treatment Period.
²⁵ AEs that occur within 2 weeks prior to Day 1 and prior to dosing on Day 1 will be considered baseline signs/symptoms. AEs occurring or worsening after the dose on Day 1 through the end of the Treatment Period will be considered treatment-emergent AEs (TEAEs). AEs occurring or worsening during the Follow-Up Period will be considered postdose AEs. All SAEs that occur from the signing of informed consent through 30 days after study discontinuation will be recorded.
²⁶ PK samples will be collected prior to, and at the end of, the infusion on Day 1 and Weeks 3 and 12 of the Treatment Period and a single sample will be collected at Week 24.
²⁷ Subjects at one clinical investigative site will undergo MRI on Day 1 and at the Week 24 visit.

### Example 32

### Half-Life Extended AVE1642

The AVE1642 monoclonal antibody (humanized form of murine antibody EM164) is described in Example 4. A half-life extended version, using amino acid substitutions in the Fc portion of the antibody (i.e., an Fc variant), can also be used for the treatment of TED, as described in Example 31.

For instance, an AVE1642 humanized monoclonal antibody with an HCDR1 comprising SYWMH (SEQ ID NO:25), an HCDR2 comprising EINPSNGRTN (SEQ ID NO:76), an HCDR3 comprising GRPDYYGSSKWYFDV (SEQ ID NO:27), an LCDR1 comprising RSSQSIVHSNVNTYLE (SEQ ID NO:28), an LCDR2 comprising KVSNRFS (SEQ ID NO:29), and an LCDR3 comprising FQGSHVPPT (SEQ ID NO:30), or alternate sequences disclosed in Example 4, can further comprise a variant Fc region comprising mutations that substitute a methionine at position 428 with a leucine (Met428Leu) and substitute an asparagine at position 434 with a serine (Asn434Ser), wherein the amino acid substitution numbering is EU as in Kabat.

Likewise, an AVE1642 humanized monoclonal antibody with an HCDR1 comprising SYWMH (SEQ ID NO:25), an HCDR2 comprising EINPSNGRTN (SEQ ID NO:76), an HCDR3 comprising GRPDYYGSSKWYFDV (SEQ ID NO:27), an LCDR1 comprising RSSQSIVHSNVNTYLE (SEQ ID NO:28), an LCDR2 comprising KVSNRFS (SEQ ID NO:29), and an LCDR3 comprising FQGSHVPPT (SEQ ID NO:30), or alternate sequences disclosed in Example 4, can further comprise a variant Fc region comprising mutations that substitute a first mutation that is a tyrosine at position 252 (Met252Tyr), a second mutation that is a threonine at position 254 (Ser254Thr), and a third mutation that is a glutamic acid at position 256 (Thr256Glu), wherein the amino acid substitution numbering is EU as in Kabat.

Such a half-life extended AVE1642 antibody, comprising M428L/N434S substitutions, or comprising M252Y/S254T/T256E substitutions, would be expected to be effective at a reduced dose and/or frequency than similar antibodies described in Example 4 alone. For instance, treatment of a subject with TED and/or symptoms of proptosis and/or diplopia would be expected to be treated at a dose of about 1-5 mg/kg, or about 1-10 mg/kg, or about 1-20 mg/kg, or about 1-50 mg/kg. In some embodiments, treatment of TED with this half-life extended antibody would be expected to occur as described herein at a dose of 1 mg/kg, or 2 mg/kg, or 3 mg/kg, or 4 mg/kg, or 5 mg/kg, or 6 mg/kg, or 7 mg/kg, or 8 mg/kg, or 9 mg/kg, or 10 mg/kg.

Likewise, the AVE1642 half-life extended antibody comprising the above CDRs and M428L/N434S Fc variant would also be expected to be suitable for administration via intravenous (IV) or even subcutaneous (SC) injection.

### Example 33

### Half-Life Extended Tenrotumumab

The teprotumumab monoclonal antibody is described in Example A. A half-life extended version, using the amino acid substitutions in the Fc portion of the antibody (i.e., an Fc variant) as described herein, can also be used for the treatment of TED as described in Example 31.

For instance, a teprotumumab monoclonal antibody with an HCDR1 comprising SEQ ID NO:84, an HCDR2 comprising SEQ ID NO:85, an HCDR3 comprising SEQ ID NO:86, an LCDR1 comprising SEQ ID NO:87, an LCDR2 comprising SEQ ID NO:88, and an LCDR3 comprising SEQ ID NO:89, can further comprise a variant Fc region comprising mutations that substitute a methionine at position 428 with a leucine (Met428Leu) and substitute an asparagine at position 434 with a serine (Asn434Ser), wherein the amino acid substitution numbering is EU as in Kabat.

Likewise, a teprotumumab monoclonal antibody with an HCDR1 comprising SEQ ID NO:84, an HCDR2 comprising SEQ ID NO:85, an HCDR3 comprising SEQ ID NO:86, an LCDR1 comprising SEQ ID NO:87, an LCDR2 comprising SEQ ID NO:88, and an LCDR3 comprising SEQ ID NO:89, can further comprise a variant Fc region comprising mutations that substitute a first mutation that is a tyrosine at position 252 (Met252Tyr), a second mutation that is a threonine at position 254 (Ser254Thr), and a third mutation that is a glutamic acid at position 256 (Thr256Glu), wherein the amino acid substitution numbering is EU as in Kabat.

Such half-life extended teprotumumab antibodies, comprising either the M428L/N434S substitutions or the M252Y/S254T/T256E substitutions, would be expected to be effective at treating TED. Further, such half-life extended antibodies would be expected to require a less frequent dosing regimen as compared to the teprotumumab antibody without such Fc variants.

### Example 34

### Half-Life Extended Anti-IGF-1R Antibodies

Other antibodies disclosed herein which bind to and/or inhibit IGF-1R may be made using appropriate binding sequences, such as those disclosed in Examples 1-3 and 5-10, and comprising either M428L/N434S substitutions or M252Y/S254T/T256E substitutions in the Fc region, would be expected to be effective at treating TED. Further, such half-life extended antibodies would be expected to require a less frequent dosing regimen as compared to the antibody (e.g., teprotumumab) without such Fc variants.

### Other Embodiments

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

What is claimed is:

1. An antibody which binds to the insulin like growth factor-I receptor (IGF-1R), comprising either:
a variant Fc region comprising mutations that substitute a methionine at position 428 with a leucine (Met428Leu) and substitute an asparagine at position 434 with a serine (Asn434Ser), wherein the amino acid substitution numbering is EU as in Kabat; or
a variant Fc region comprising mutations that substitute a first mutation that is a tyrosine at position 252 (Met252Tyr), a second mutation that is a threonine at position 254 (Ser254Thr), and a third mutation that is a glutamic acid at position 256 (Thr256Glu), wherein the amino acid substitution numbering is EU as in Kabat.

2. The antibody of claim 1, wherein the antibody binds to and inhibits IGF-1R.

3. The antibody of either of claims 1-2, wherein the antibody or antigen-binding portion thereof cross-competes for binding to IGF-1R with a reference antibody or reference antigen-binding portion thereof.

4. The antibody of claim 3, wherein the reference antibody is chosen from αIR3, dalotuzumab, ganitumab, xentuzumab, AVE1642, figitumumab, dusigitumab, cituxumumab, BIIB022, robatumumab, teprotumumab, and Antibody 2.

5. The antibody of any of claims 1-4, wherein the antibody is chosen from an IgA, IgD, IgE, and IgG.

6. The antibody of claim 5, wherein the antibody is an IgG.

7. The antibody of claim 6, wherein the antibody is chosen from an IgG1, IgG2, IgG3, and IgG4.

8. The antibody of claim 7, wherein the antibody is an IgG1.

9. The antibody of any of claims 1-8, wherein the antibody:
is capable of reducing insulin like growth factor-I receptor (IGF-1R) signaling;
is capable of inhibiting thyroid stimulating hormone receptor (TSHR)/IGF-1R crosstalk (i.e., formation of a TSHR/IGF-1R signalosome);
is capable of reducing hyaluronan (HA) secretion in orbital fibroblasts;
is capable of persisting for an extended period of time in vivo (i.e., has a longer half-life) compared to an antibody that does not comprise the M428L/N434S or M252Y/S254T/T256E substitutions; and/or
is capable of being dosed less frequently, or in a lower amount per dose, compared to an antibody that does not comprise the M428L/N434S or M252Y/S254T/T256E substitutions.

10. The antibody of any of claims 1-9, wherein the antibody comprises complementarity determining regions (CDRs) derived from an antibody chosen from dalotuzumab, ganitumab, xentuzumab, AVE1642, figitumumab, dusigitumab, cituxumumab, BIIB022, robatumumab, teprotumumab, and Antibody 2.

11. The antibody of any of claims 1-10 wherein the antibody comprises a heavy chain variable region that comprises HCDR1, HCDR2, and HCDR3 domains; and a light chain variable region that comprises LCDR1, LCDR2, and LCDR3 domains, comprising:
a HCDR1 comprising the amino acid sequence of SEQ ID NO:1, a HCDR2 comprising the amino acid sequence of SEQ ID NO:2, a HCDR3 comprising the amino acid sequence of SEQ ID NO:3, a LCDR1 comprising the amino acid sequence of SEQ ID NO:4, a LCDR2 comprising the amino acid sequence of SEQ ID NO:5, and a LCDR3 comprising the amino acid sequence of SEQ ID NO:6;
a HCDR1 comprising the amino acid sequence of SEQ ID NO:9, a HCDR2 comprising the amino acid sequence of SEQ ID NO:10, a HCDR3 comprising the amino acid sequence of SEQ ID NO:11, a LCDR1 comprising the amino acid sequence of SEQ ID NO:12, a LCDR2 comprising the amino acid sequence of SEQ ID NO:13, and a LCDR3 comprising the amino acid sequence of SEQ ID NO:14;
a HCDR1 comprising the amino acid sequence of SEQ ID NO:17, a HCDR2 comprising the amino acid sequence of SEQ ID NO:18, a HCDR3 comprising the amino acid sequence of SEQ ID NO:19, a LCDR1 comprising the amino acid sequence of SEQ ID NO:20, a LCDR2 comprising the amino acid sequence of SEQ ID NO:21, and a LCDR3 comprising the amino acid sequence of SEQ ID NO:22;
a HCDR1 comprising HCDR1 comprising the amino acid sequence of SEQ ID NO:25 or SEQ ID NO:75, a HCDR2 comprising the amino acid sequence of SEQ ID NO:26 or SEQ ID NO:76 or SEQ ID NO:77, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30;
a HCDR1 comprising the amino acid sequence of SEQ ID NO:33, a HCDR2 comprising the amino acid sequence of SEQ ID NO:34, a HCDR3 comprising the amino acid sequence of SEQ ID NO:35, a LCDR1 comprising the amino acid sequence of SEQ ID NO:36, a LCDR2 comprising the amino acid sequence of SEQ ID NO:37, and a LCDR3 comprising the amino acid sequence of SEQ ID NO:38;
a HCDR1 comprising the amino acid sequence of SEQ ID NO:41, a HCDR2 comprising the amino acid sequence of SEQ ID NO:42, a HCDR3 comprising the amino acid sequence of SEQ ID NO:43, a LCDR1 comprising the amino acid sequence of SEQ ID NO:44, a LCDR2 comprising the amino acid sequence of SEQ ID NO:45, and a LCDR3 comprising the amino acid sequence of SEQ ID NO:46;
a HCDR1 comprising the amino acid sequence of SEQ ID NO:49, a HCDR2 comprising the amino acid sequence of SEQ ID NO:50, a HCDR3 comprising the amino acid sequence of SEQ ID NO:51, a LCDR1 comprising the amino acid sequence of SEQ ID NO:52, a LCDR2 comprising the amino acid sequence of SEQ ID NO:53, and a LCDR3 comprising the amino acid sequence of SEQ ID NO:54;
a HCDR1 comprising the amino acid sequence of SEQ ID NO:57, a HCDR2 comprising the amino acid sequence of SEQ ID NO:58, a HCDR3 comprising the amino acid sequence of SEQ ID NO:59, a LCDR1 comprising the amino acid sequence of SEQ ID NO:60, a LCDR2 comprising the amino acid sequence of SEQ ID NO:61, and a LCDR3 comprising the amino acid sequence of SEQ ID NO:62;
a HCDR1 comprising the amino acid sequence of SEQ ID NO:84, a HCDR2 comprising the amino acid sequence of SEQ ID NO:85, a HCDR3 comprising the amino acid sequence of SEQ ID NO:86, a LCDR1 comprising the amino acid sequence of SEQ ID NO:87, a LCDR2 comprising the amino acid sequence of SEQ ID NO:88, and a LCDR3 comprising the amino acid sequence of SEQ ID NO:89; or
a HCDR1 comprising the amino acid sequence of SEQ ID NO:84, a HCDR2 comprising the amino acid sequence of SEQ ID NO:92, a HCDR3 comprising the amino acid sequence of SEQ ID NO:86, a LCDR1 comprising the amino acid sequence of SEQ ID NO:87, a LCDR2 comprising the amino acid sequence of SEQ ID NO:93, and a LCDR3 comprising the amino acid sequence of SEQ ID NO:89.

12. The antibody of any of claims 1-9, wherein the antibody comprises heavy chain variable domain (V_{H}) and a light chain variable domain (V_{L}) derived from an antibody chosen from dalotuzumab, ganitumab, xentuzumab, AVE1642, figitumumab, dusigitumab, cituxumumab, BIIB022, robatumumab, teprotumumab, and Antibody 2.

13. The antibody of any of claims 1-9, comprising:
a heavy chain variable domain comprising the amino acid sequence of SEQ ID NO:7, and a light chain variable domain comprising the amino acid sequence of SEQ ID NO:8;
a heavy chain variable domain comprising the amino acid sequence of SEQ ID NO:15, and a light chain variable domain comprising the amino acid sequence of SEQ ID NO:16;
a heavy chain variable domain comprising the amino acid sequence of SEQ ID NO:23, and a light chain variable domain comprising the amino acid sequence of SEQ ID NO:24;
a heavy chain variable domain comprising the amino acid sequence of SEQ ID NO:31 or 78 or 79, and a light chain variable domain comprising the amino acid sequence of SEQ ID NO:32 or 80 or 81 or 82 or 83;
a heavy chain variable domain comprising the amino acid sequence of SEQ ID NO:39, and a light chain variable domain comprising the amino acid sequence of SEQ ID NO:40;
a heavy chain variable domain comprising the amino acid sequence of SEQ ID NO:47, and a light chain variable domain comprising the amino acid sequence of SEQ ID NO:48;
a heavy chain variable domain comprising the amino acid sequence of SEQ ID NO:55, and a light chain variable domain comprising the amino acid sequence of SEQ ID NO:56;
a heavy chain variable domain comprising the amino acid sequence of SEQ ID NO:63, and a light chain variable domain comprising the amino acid sequence of SEQ ID NO:64; or
a heavy chain variable domain comprising the amino acid sequence of SEQ ID NO:65, and a light chain variable domain comprising the amino acid sequence of SEQ ID NO:66;
a heavy chain variable domain comprising the amino acid sequence of SEQ ID NO:90, and a light chain variable domain comprising the amino acid sequence of SEQ ID NO:91;
a heavy chain variable domain comprising the amino acid sequence of SEQ ID NO:94, or and a light chain variable domain comprising the amino acid sequence of SEQ ID NO:95.

14. The antibody of any of claims 1-10, comprising a HCDR1 comprising HCDR1 comprising the amino acid sequence of SEQ ID NO:25 or SEQ ID NO:75, a HCDR2 comprising the amino acid sequence of SEQ ID NO:26 or SEQ ID NO:76 or SEQ ID NO:77, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30.

15. The antibody of any of claims 1-9, comprising a HCDR1 comprising the amino acid sequence of SEQ ID NO:25, a HCDR2 comprising the amino acid sequence of SEQ ID NO:76, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30.

16. The antibody of any of claims 1-9, comprising
a HCDR1 comprising the amino acid sequence of SEQ ID NO:25, a HCDR2 comprising the amino acid sequence of SEQ ID NO:76, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30;
a HCDR1 comprising the amino acid sequence of SEQ ID NO:25, a HCDR2 comprising the amino acid sequence of SEQ ID NO:77, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30;
a HCDR1 comprising the amino acid sequence of SEQ ID NO:25, a HCDR2 comprising the amino acid sequence of SEQ ID NO:26, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30;
a HCDR1 comprising the amino acid sequence of SEQ ID NO:75, a HCDR2 comprising the amino acid sequence of SEQ ID NO:76, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30;
a HCDR1 comprising the amino acid sequence of SEQ ID NO:75, a HCDR2 comprising the amino acid sequence of SEQ ID NO:77, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30; or
a HCDR1 comprising the amino acid sequence of SEQ ID NO:75, a HCDR2 comprising the amino acid sequence of SEQ ID NO:26, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30.

17. The antibody of any of claims 1-9, wherein the antibody comprises a heavy chain variable domain comprising the amino acid sequence of SEQ ID NO:31 or 78 or 79, and a light chain variable domain comprising the amino acid sequence of SEQ ID NO:32 or 80 or 81 or 82 or 83.

18. The antibody of claim 17, wherein the antibody comprises a heavy chain variable domain comprising the amino acid sequence of SEQ ID NO:78 and a light chain variable domain comprising the amino acid sequence of SEQ ID NOs:80 or 81 or 82 or 83.

19. The antibody of claim 18, wherein the antibody comprises the light chain variable domain comprising the amino acid sequence of SEQ ID NO:80.

20. The antibody of claim 19, wherein the antibody comprises the light chain variable domain comprising the amino acid sequence of SEQ ID NO:81.

21. The antibody of claim 19, wherein the antibody comprises the light chain variable domain comprising the amino acid sequence of SEQ ID NO:82.

22. The antibody of claim 19, wherein the antibody comprises the light chain variable domain comprising the amino acid sequence of SEQ ID NO:83.

23. The antibody of claim 18, wherein the antibody comprises a heavy chain variable domain comprising the amino acid sequence of SEQ ID NO:31 and a light chain variable domain comprising the amino acid sequence of SEQ ID NOs:80 or 81 or 82 or 83.

24. The antibody of claim 23, wherein the antibody comprises the light chain variable domain comprising the amino acid sequence of SEQ ID NO:80.

25. The antibody of claim 23, wherein the antibody comprises the light chain variable domain comprising the amino acid sequence of SEQ ID NO:81.

26. The antibody of claim 23, wherein the antibody comprises the light chain variable domain comprising the amino acid sequence of SEQ ID NO:82.

27. The antibody of claim 23, wherein the antibody comprises the light chain variable domain comprising the amino acid sequence of SEQ ID NO:83.

28. The antibody of any of claims 1-9, comprising a HCDR1 comprising the amino acid sequence of SEQ ID NO:84, a HCDR2 comprising the amino acid sequence of SEQ ID NO:85, a HCDR3 comprising the amino acid sequence of SEQ ID NO:86, a LCDR1 comprising the amino acid sequence of SEQ ID NO:87, a LCDR2 comprising the amino acid sequence of SEQ ID NO:88, and a LCDR3 comprising the amino acid sequence of SEQ ID NO:89.

29. The antibody of any of claims 1-9, wherein the antibody comprises a heavy chain variable domain comprising SEQ ID NO:90 and a light chain variable domain comprising SEQ ID NO:91.

30. The antibody of any of claims 1-29, wherein the antibody comprises a variant Fc region comprising mutations that substitute a methionine at position 428 with a leucine (Met428Leu) and substitute an asparagine at position 434 with a serine (Asn434Ser), wherein the amino acid substitution numbering is EU as in Kabat.

31. The antibody of any of claims 1-29, wherein the antibody comprises a variant Fc region comprising mutations that substitute a first mutation that is a tyrosine at position 252 (Met252Tyr), a second mutation that is a threonine at position 254 (Ser254Thr), and a third mutation that is a glutamic acid at position 256 (Thr256Glu), wherein the amino acid substitution numbering is EU as in Kabat.

32. An antibody which binds to the insulin like growth factor-I receptor (IGF-1R), wherein the antibody comprises a heavy chain variable region that comprises HCDR1, HCDR2, and HCDR3 domains; and a light chain variable region that comprises LCDR1, LCDR2, and LCDR3 domains, comprising:
a HCDR1 comprising HCDR1 comprising the amino acid sequence of SEQ ID NO:25 or SEQ ID NO:75, a HCDR2 comprising the amino acid sequence of SEQ ID NO:26 or SEQ ID NO:76 or SEQ ID NO:77, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30; and either
a variant Fc region comprising mutations that substitute a methionine at position 428 with a leucine (Met428Leu) and substitute an asparagine at position 434 with a serine (Asn434Ser), wherein the amino acid substitution numbering is EU as in Kabat; or
a variant Fc region comprising mutations that substitute a first mutation that is a tyrosine at position 252 (Met252Tyr), a second mutation that is a threonine at position 254 (Ser254Thr), and a third mutation that is a glutamic acid at position 256 (Thr256Glu), wherein the amino acid substitution numbering is EU as in Kabat.

33. An antibody which binds to the insulin like growth factor-I receptor (IGF-1R), wherein the antibody comprises a heavy chain variable region that comprises HCDR1, HCDR2, and HCDR3 domains; and a light chain variable region that comprises LCDR1, LCDR2, and LCDR3 domains, comprising:
a HCDR1 comprising the amino acid sequence of SEQ ID NO:25, a HCDR2 comprising the amino acid sequence of SEQ ID NO:76, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30; and either
a variant Fc region comprising mutations that substitute a methionine at position 428 with a leucine (Met428Leu) and substitute an asparagine at position 434 with a serine (Asn434Ser), wherein the amino acid substitution numbering is EU as in Kabat; or
a variant Fc region comprising mutations that substitute a first mutation that is a tyrosine at position 252 (Met252Tyr), a second mutation that is a threonine at position 254 (Ser254Thr), and a third mutation that is a glutamic acid at position 256 (Thr256Glu), wherein the amino acid substitution numbering is EU as in Kabat.

34. An antibody which binds to the insulin like growth factor-I receptor (IGF-1R), wherein the antibody comprises a heavy chain variable region that comprises HCDR1, HCDR2, and HCDR3 domains; and a light chain variable region that comprises LCDR1, LCDR2, and LCDR3 domains, comprising:
a HCDR1 comprising the amino acid sequence of SEQ ID NO:25, a HCDR2 comprising the amino acid sequence of SEQ ID NO:76, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30;
a HCDR1 comprising the amino acid sequence of SEQ ID NO:25, a HCDR2 comprising the amino acid sequence of SEQ ID NO:77, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30;
a HCDR1 comprising the amino acid sequence of SEQ ID NO:25, a HCDR2 comprising the amino acid sequence of SEQ ID NO:26, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30;
a HCDR1 comprising the amino acid sequence of SEQ ID NO:75, a HCDR2 comprising the amino acid sequence of SEQ ID NO:76, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30;
a HCDR1 comprising the amino acid sequence of SEQ ID NO:75, a HCDR2 comprising the amino acid sequence of SEQ ID NO:77, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30; or
a HCDR1 comprising the amino acid sequence of SEQ ID NO:75, a HCDR2 comprising the amino acid sequence of SEQ ID NO:26, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30; and either
a variant Fc region comprising mutations that substitute a methionine at position 428 with a leucine (Met428Leu) and substitute an asparagine at position 434 with a serine (Asn434Ser), wherein the amino acid substitution numbering is EU as in Kabat; or
a variant Fc region comprising mutations that substitute a first mutation that is a tyrosine at position 252 (Met252Tyr), a second mutation that is a threonine at position 254 (Ser254Thr), and a third mutation that is a glutamic acid at position 256 (Thr256Glu), wherein the amino acid substitution numbering is EU as in Kabat.

35. An antibody which binds to the insulin like growth factor-I receptor (IGF-1R), wherein the antibody comprises:
a heavy chain variable domain comprising the amino acid sequence of SEQ ID NO:31 or 78 or 79, and a light chain variable domain comprising the amino acid sequence of SEQ ID NO:32 or 80 or 81 or 82 or 83; and either
a variant Fc region comprising mutations that substitute a methionine at position 428 with a leucine (Met428Leu) and substitute an asparagine at position 434 with a serine (Asn434Ser), wherein the amino acid substitution numbering is EU as in Kabat; or
a variant Fc region comprising mutations that substitute a first mutation that is a tyrosine at position 252 (Met252Tyr), a second mutation that is a threonine at position 254 (Ser254Thr), and a third mutation that is a glutamic acid at position 256 (Thr256Glu), wherein the amino acid substitution numbering is EU as in Kabat.

36. The antibody of claim 35, wherein the antibody comprises a heavy chain variable domain comprising the amino acid sequence of SEQ ID NO:78 and a light chain variable domain comprising the amino acid sequence of SEQ ID NOs:80 or 81 or 82 or 83.

37. The antibody of claim 36, wherein the antibody comprises the light chain variable domain comprising the amino acid sequence of SEQ ID NO:80.

38. The antibody of claim 36, wherein the antibody comprises the light chain variable domain comprising the amino acid sequence of SEQ ID NO:81.

39. The antibody of claim 36, wherein the antibody comprises the light chain variable domain comprising the amino acid sequence of SEQ ID NO:82.

40. The antibody of claim 36, wherein the antibody comprises the light chain variable domain comprising the amino acid sequence of SEQ ID NO:83.

41. The antibody of claim 35, wherein the antibody comprises a heavy chain variable domain comprising the amino acid sequence of SEQ ID NO:31 and a light chain variable domain comprising the amino acid sequence of SEQ ID NOs:80 or 81 or 82 or 83.

42. The antibody of claim 41, wherein the antibody comprises the light chain variable domain comprising the amino acid sequence of SEQ ID NO:80.

43. The antibody of claim 41, wherein the antibody comprises the light chain variable domain comprising the amino acid sequence of SEQ ID NO:81.

44. The antibody of claim 41, wherein the antibody comprises the light chain variable domain comprising the amino acid sequence of SEQ ID NO:82.

45. The antibody of claim 41, wherein the antibody comprises the light chain variable domain comprising the amino acid sequence of SEQ ID NO:83.

46. An antibody which binds to the insulin like growth factor-I receptor (IGF-1R), wherein the antibody comprises a heavy chain variable region that comprises HCDR1, HCDR2, and HCDR3 domains; and a light chain variable region that comprises LCDR1, LCDR2, and LCDR3 domains, comprising:
a HCDR1 comprising the amino acid sequence of SEQ ID NO:84, a HCDR2 comprising the amino acid sequence of SEQ ID NO:85, a HCDR3 comprising the amino acid sequence of SEQ ID NO:86, a LCDR1 comprising the amino acid sequence of SEQ ID NO:87, a LCDR2 comprising the amino acid sequence of SEQ ID NO:88, and a LCDR3 comprising the amino acid sequence of SEQ ID NO:89; and either
a variant Fc region comprising mutations that substitute a methionine at position 428 with a leucine (Met428Leu) and substitute an asparagine at position 434 with a serine (Asn434Ser), wherein the amino acid substitution numbering is EU as in Kabat; or
a variant Fc region comprising mutations that substitute a first mutation that is a tyrosine at position 252 (Met252Tyr), a second mutation that is a threonine at position 254 (Ser254Thr), and a third mutation that is a glutamic acid at position 256 (Thr256Glu), wherein the amino acid substitution numbering is EU as in Kabat.

47. An antibody which binds to the insulin like growth factor-I receptor (IGF-1R), wherein the antibody comprises:
a heavy chain variable domain comprising SEQ ID NO:7 and a light chain variable domain comprising SEQ ID NO:8; and either
a variant Fc region comprising mutations that substitute a methionine at position 428 with a leucine (Met428Leu) and substitute an asparagine at position 434 with a serine (Asn434Ser), wherein the amino acid substitution numbering is EU as in Kabat; or
a variant Fc region comprising mutations that substitute a first mutation that is a tyrosine at position 252 (Met252Tyr), a second mutation that is a threonine at position 254 (Ser254Thr), and a third mutation that is a glutamic acid at position 256 (Thr256Glu), wherein the amino acid substitution numbering is EU as in Kabat.

48. The antibody of any of claims 32-47, wherein the antibody comprises a variant Fc region comprising mutations that substitute a methionine at position 428 with a leucine (Met428Leu) and substitute an asparagine at position 434 with a serine (Asn434Ser), wherein the amino acid substitution numbering is EU as in Kabat.

49. The antibody of any of claims 32-47, wherein the antibody comprises a variant Fc region comprising mutations that substitute a first mutation that is a tyrosine at position 252 (Met252Tyr), a second mutation that is a threonine at position 254 (Ser254Thr), and a third mutation that is a glutamic acid at position 256 (Thr256Glu), wherein the amino acid substitution numbering is EU as in Kabat.

50. A nucleotide sequence encoding the polypeptide sequence of the antibody of any of claims 1-49.

51. An expression vector comprising the nucleotide sequence of claim 50.

52. A Chinese hamster ovary (CHO) cell line expressing the vector of claim 51.

53. A pharmaceutical composition comprising a therapeutically effective amount of the antibody as recited in any of claims 1-49, and a pharmaceutically acceptable carrier.

54. The pharmaceutical composition of claim 53, wherein the therapeutically effective amount comprises a dosage of 1-10 mg/kg.

55. The pharmaceutical composition of claim 54, wherein the therapeutically effective amount comprises a dosage of 1-5 mg/kg.

56. The pharmaceutical composition of claim 55, wherein the therapeutically effective amount comprises a dosage of about 2 mg/kg, about 3 mg/kg, about 4 mg/kg, or about 5 mg/kg.

57. The pharmaceutical composition of any of claims 53-56, wherein the therapeutically effective amount is formulated for administration every 1, 2, 3, 4, or 5 weeks (i.e., QW, Q2W, Q3W, Q4W, or Q5W).

58. The pharmaceutical composition of any of claims 53-57, wherein the pharmaceutically acceptable carrier is suitable for intravenous (IV) or subcutaneous (SC) administration.

59. The pharmaceutical composition of claim 53, comprising a therapeutically effective amount of the antibody as recited in any of claims 14-27 and 32-45, wherein the therapeutically effective amount comprises a dosage of:
1-60 mg/kg or 75-4500 mg; or
0.6-40 mg/kg or 45-3000 mg; or
0.3-20 mg/kg or 22-1500 mg.

60. The pharmaceutical composition of claim 59, formulated for IV administration.

61. The pharmaceutical composition of claim 60, formulated for dosing every 4, 3, 2, or 1 weeks.

62. The pharmaceutical composition of claim 53, comprising a therapeutically effective amount of the antibody as recited in any of claims 14-27 and 32-45, wherein the therapeutically effective amount comprises a dosage of:
1-30 mg/kg or 75-2250 mg;
0.6-20 mg/kg or 1500 mg;
0.3-10 mg/kg or 750 mg.

63. The pharmaceutical composition of claim 53, comprising a therapeutically effective amount of the antibody as recited in any of claims 14-27 and 32-45, wherein the therapeutically effective amount comprises a dosage of:
1-20 mg/kg or 75-1500 mg;
0.6-13.5 mg/kg or 1000 mg;
0.3-7 mg/kg or 500 mg.

64. The pharmaceutical composition of any of claims 62-63, formulated for subcutaneous administration.

65. The pharmaceutical composition of claim 64, formulated for dosing every 4, 3, 2, or 1 weeks.

66. An autoinjector comprising the pharmaceutical formulation as recited in claim 64.

67. A method of treating thyroid eye disease (TED) in a subject with TED, comprising administering to the subject a therapeutically effective amount of the antibody of any of claims 1-49 or the pharmaceutical composition of any of claims 53-65.

68. A method of reducing proptosis in a subject with thyroid eye disease (TED), comprising administering to the subject a therapeutically effective amount of the antibody of any of claims 1-49 or the pharmaceutical composition of any of claims 53-65.

69. The method of claim 68, wherein proptosis is reduced by at least 2 mm.

70. The method of claim 69, wherein proptosis is reduced by at least 3 mm.

71. The method of claim 70, wherein proptosis is reduced by at least 4 mm.

72. The method of claim 68, wherein the method additionally comprises reducing the clinical activity score (CAS) in the subject with TED.

73. The method of claim 72, wherein proptosis is reduced by at least 2 mm and CAS is reduced by at least 2 points.

74. The method of claim 73, wherein CAS is reduced by at least 3 points.

75. The method of claim 74, wherein proptosis is reduced by at least 3 mm and CAS is reduced by at least 3 points.

76. A method of treating or reducing the severity of diplopia in a subject with thyroid eye disease (TED), comprising administering to the subject a therapeutically effective amount of the antibody of any of claims 1-49 or the pharmaceutical composition of any of claims 53-65.

77. The method of claim 76, wherein the diplopia is constant diplopia.

78. The method of claim 76, wherein the diplopia is intermittent diplopia.

79. The method of claim 76, wherein the diplopia is inconstant diplopia.

80. The method of claim 76, wherein the improvement in or reduction in severity of diplopia is sustained at least 20 weeks after discontinuation of inhibitor administration.

81. The method of claim 80, wherein the improvement in or reduction in severity of diplopia is sustained at least 50 weeks after discontinuation of inhibitor administration.

82. A method of reducing Clinical Activity Score (CAS) of thyroid eye disease (TED) in a subject with TED, comprising administering to a subject in need thereof a therapeutically effective amount of the antibody of any of claims 1-49 or the pharmaceutical composition of any of claims 53-65.

83. The method of claim 82, wherein CAS is reduced by at least 2 points.

84. The method of claim 83, wherein CAS is reduced by at least 3 points.

85. A method of:
reducing insulin like growth factor-I receptor (IGF-1R) signaling;
inhibiting thyroid stimulating hormone receptor (TSHR)/IGF-1R crosstalk (i.e., formation of a TSHR/IGF-1R signalosome); and/or
reducing hyaluronan (HA) secretion in orbital fibroblasts;
in a subject with TED, comprising administering to a subject in need thereof a therapeutically effective amount of the antibody of any of claims 1-49 or the pharmaceutical composition of any of claims 53-65,
wherein the antibody persists for an extended period of time in vivo (i.e., has a longer half-life) compared to an antibody that does not comprise the M428L/N434S or M252Y/S254T/T256E substitutions; and/or
wherein the antibody or pharmaceutical composition is administered less frequently, or in a lower amount per dose, compared to an antibody that does not comprise the M428L/N434S or M252Y/S254T/T256E substitutions.

86. The method of any of claims 67-85, wherein the therapeutically effective amount comprises a dosage of 1-10 mg/kg.

87. The method of claim 86, wherein the therapeutically effective amount comprises a dosage of 1-5 mg/kg.

88. The method of claim 87, wherein the therapeutically effective amount comprises a dosage of about 2 mg/kg, about 3 mg/kg, about 4 mg/kg, or about 5 mg/kg.

89. The method of any of claims 67-88, wherein the therapeutically effective amount is administered every 1, 2, 3, 4, or 5 weeks (i.e., QW, Q2W, Q3W, Q4W, or Q5W).

90. The method of any of claims 67-89, wherein the therapeutically effective amount is administered intravenously (IV) or subcutaneously (SC).

91. The method of any of claims 67-85, comprising a therapeutically effective amount of the antibody as recited in any of claims 14-27 and 32-45, wherein the therapeutically effective amount comprises a dosage of
1-5 mg/kg or 75-375 mg IV Q3W; or
0.6-4 mg/kg or 45-300 mg IV Q2W; or
0.3-3 mg/kg or 22-225 mg IV QW.

92. The method of claim 91, formulated for IV administration.

93. The method of claim 92, formulated for dosing every 4, 3, 2, or 1 weeks.

94. The method of any of claims 67-85, comprising a therapeutically effective amount of the antibody as recited in any of claims 14-27 and 32-45, wherein the therapeutically effective amount comprises a dosage of:
1-30 mg/kg or 75-2250 mg;
0.6-20 mg/kg or 1500 mg;
0.3-10 mg/kg or 750 mg.

95. The method of any of claims 67-85, comprising a therapeutically effective amount of the antibody as recited in any of claims 14-27 and 32-45, wherein the therapeutically effective amount comprises a dosage of:
1-20 mg/kg or 75-1500 mg;
0.6-13.5 mg/kg or 1000 mg;
0.3-7 mg/kg or 500 mg.

96. The method of any of claims 94-95, formulated for subcutaneous administration.

97. The method of claim 96, formulated for dosing every 4, 3, 2, or 1 weeks.

98. The method of claim 95, wherein the subcutaneous administration is done using an autoinjector.

99. The use of the antibody of any of claims 1-49, or the pharmaceutical composition of any of claims 53-65 or the autoinjector of claim 66, for the treatment of thyroid eye disease (TED) in a subject with TED, reduction of proptosis in a subject with TED, reduction of the severity of diplopia in a subject with TED, or reduction of CAS in a subject with TED, as recited in any of claims 67-99.

100. The use of the antibody of any of claims 1-49, or the pharmaceutical composition of any of claims 53-65 or the autoinjector of claim 66, in the manufacture of a medicament for the treatment of thyroid eye disease (TED) in a subject with TED, reduction of proptosis in a subject with TED, reduction of the severity of diplopia in a subject with TED, or reduction of CAS in a subject with TED, as recited in any of claims 67-99.
